SEQUENCE LISTING (57 sequences)

SEQ ID NO: 1 - protein, 152 aa, Homo sapiens
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 2 - protein, 106 aa, Homo sapiens
VNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLE

SEQ ID NO: 3 - protein, 106 aa, Homo sapiens
VNCSNMIDEIITHLKQPPLPLLDFNNLNGEDQDILMENNLRRPNLEAFNRAVKSLQNASAIESILKNLLPCLPLATAAPTRHPIHIKDGDWNEFRRELTFYLKTLE

SEQ ID NO: 4 - protein, 106 aa, Homo sapiens
VNCSNMIDEIITHLKQPPLPLLDFNNLNGEDQDILMENNLRRPNLEAFNRAVKSLQNASAIESILKNLLPCLPLATAAPTRHPIHIKAGDWNEFRRKLTFYLVTLE

SEQ ID NO: 5 - protein, 106 aa, Homo sapiens
VNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLE

SEQ ID NO: 6 - protein, 5 aa, synthetic construct
  REGION 1..5: note = Synthetic
GGGGS

SEQ ID NO: 7 - protein, 10 aa, synthetic construct
  REGION 1..10: note = Synthetic
GGGGSGGGGS

SEQ ID NO: 8 - protein, 45 aa, synthetic construct
  REGION 1..45: note = Synthetic
TTTPAPRPPTPAPTIASQPLSLRPEACRPAAGGAVHTRGLDFACD

SEQ ID NO: 9 - protein, 42 aa, synthetic construct
  REGION 1..42: note = Synthetic
AAAIEVMYPPPYLDNEKSNGTIIHVKGKHLCPSPLFPGPSKP

SEQ ID NO: 10 - protein, 229 aa, synthetic construct
  REGION 1..229: note = Synthetic
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, synthetic construct
  REGION 1..229: note = Synthetic
ESKYGPPCPSCPAPEFEGGPSVFLFPPKPKDTLMISRTPEVTCVVVDVSQEDPEVQFNWYVDGVEVHQAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 24 aa, synthetic construct
  REGION 1..24: note = Synthetic
IYIWAPLAGTCGVLLLSLVITLYC

SEQ ID NO: 13 - protein, 68 aa, synthetic construct
  REGION 1..68: note = Synthetic
FWVLVVVGGVLACYSLLVTVAFIIFWVRSKRSRLLHSDYMNMTPRRPGPTRKHYQPYAPPRDFAAYRS

SEQ ID NO: 14 - protein, 22 aa, synthetic construct
  REGION 1..22: note = Synthetic
MALIVLGGVAGLLLFIGLFIFF

SEQ ID NO: 15 - protein, 112 aa, synthetic construct
  REGION 1..112: note = Synthetic
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 112 aa, synthetic construct
  REGION 1..112: note = Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 42 aa, synthetic construct
  REGION 1..42: note = Synthetic
KRGRKKLLYIFKQPFMRPVQTTQEEDGCSCRFPEEEEGGCEL

SEQ ID NO: 18 - protein, 41 aa, synthetic construct
RSKRSRLLHSDYMNMTPRRPGPTRKHQYPYAPPRDFAAYRS

SEQ ID NO: 19 - protein, 21 aa, synthetic construct
  REGION 1..21: note = Synthetic
MLLLVTSLLLCELPHPAFLLI

SEQ ID NO: 20 - DNA, 402 bp, synthetic construct
  misc_feature 1..402: note = Synthetic
  source 1..402: mol_type = other DNA
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcg

SEQ ID NO: 21 - protein, 469 aa, synthetic construct
  REGION 1..469: note = Synthetic
VNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLEESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - DNA, 1407 bp, synthetic construct
  misc_feature 1..1407: note = Synthetic
  source 1..1407: mol_type = other DNA
gtgaactgcagcaacatgatcgacgagatcatcacccacctgaagcagccccccctgcccctgctggacttcaacaacctgaacggcgaggaccaggacatcctgatggagaacaacctgaggaggcccaacctggaggccttcaacagggccgtgaagagcctgcagaacgccagcgccatcgagagcatcctgaagaacctgctgccctgcctgcccctggccaccgccgcccccaccaggcaccccatccacatcaaggacggcgactggaacgagttcaggaggaagctgaccttctacctgaagaccctggaggagagcaagtacggccccccctgccccccctgccccgcccccgagttcctgggcggccccagcgtgttcctgttcccccccaagcccaaggacaccctgatgatcagcaggacccccgaggtgacctgcgtggtggtggacgtgagccaggaggaccccgaggtgcagttcaactggtacgtggacggcgtggaggtgcacaacgccaagaccaagcccagggaggagcagttcaacagcacctacagggtggtgagcgtgctgaccgtgctgcaccaggactggctgaacggcaaggagtacaagtgcaaggtgagcaacaagggcctgcccagcagcatcgagaagaccatcagcaaggccaagggccagcccagggagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaggctgaccgtggacaagagcaggtggcaggagggcaacgtgttcagctgcagcgtgatgcacgaggccctgcacaaccactacacccagaagagcctgagcctgagcctgggcaagatggccctgatcgtgctgggcggcgtggccggcctgctgctgttcatcggcctgggcatcttcttcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 23 - protein, 510 aa, synthetic construct
  REGION 1..510: note = Synthetic
VNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLEESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRSKRSRLLHSDYMNMTPRRPGPTRKHQYPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - DNA, 1530 bp, synthetic construct
  misc_feature 1..1530: note = Synthetic
  source 1..1530: mol_type = other DNA
gtgaactgcagcaacatgatcgacgagatcatcacccacctgaagcagccccccctgcccctgctggacttcaacaacctgaacggcgaggaccaggacatcctgatggagaacaacctgaggaggcccaacctggaggccttcaacagggccgtgaagagcctgcagaacgccagcgccatcgagagcatcctgaagaacctgctgccctgcctgcccctggccaccgccgcccccaccaggcaccccatccacatcaaggacggcgactggaacgagttcaggaggaagctgaccttctacctgaagaccctggaggagagcaagtacggccccccctgccccccctgccccgcccccgagttcctgggcggccccagcgtgttcctgttcccccccaagcccaaggacaccctgatgatcagcaggacccccgaggtgacctgcgtggtggtggacgtgagccaggaggaccccgaggtgcagttcaactggtacgtggacggcgtggaggtgcacaacgccaagaccaagcccagggaggagcagttcaacagcacctacagggtggtgagcgtgctgaccgtgctgcaccaggactggctgaacggcaaggagtacaagtgcaaggtgagcaacaagggcctgcccagcagcatcgagaagaccatcagcaaggccaagggccagcccagggagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaggctgaccgtggacaagagcaggtggcaggagggcaacgtgttcagctgcagcgtgatgcacgaggccctgcacaaccactacacccagaagagcctgagcctgagcctgggcaagatggccctgatcgtgctgggcggcgtggccggcctgctgctgttcatcggcctgggcatcttcttcaggagcaagaggagcaggctgctgcacagcgactacatgaacatgacccccaggaggcccggccccaccaggaagcaccagtacccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 25 - protein, 511 aa, synthetic construct
  REGION 1..511: note = Synthetic
VNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLEESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - DNA, 1533 bp, synthetic construct
  misc_feature 1..1533: note = Synthetic
  source 1..1533: mol_type = other DNA
gtgaactgcagcaacatgatcgacgagatcatcacccacctgaagcagccccccctgcccctgctggacttcaacaacctgaacggcgaggaccaggacatcctgatggagaacaacctgaggaggcccaacctggaggccttcaacagggccgtgaagagcctgcagaacgccagcgccatcgagagcatcctgaagaacctgctgccctgcctgcccctggccaccgccgcccccaccaggcaccccatccacatcaaggacggcgactggaacgagttcaggaggaagctgaccttctacctgaagaccctggaggagagcaagtacggccccccctgccccccctgccccgcccccgagttcctgggcggccccagcgtgttcctgttcccccccaagcccaaggacaccctgatgatcagcaggacccccgaggtgacctgcgtggtggtggacgtgagccaggaggaccccgaggtgcagttcaactggtacgtggacggcgtggaggtgcacaacgccaagaccaagcccagggaggagcagttcaacagcacctacagggtggtgagcgtgctgaccgtgctgcaccaggactggctgaacggcaaggagtacaagtgcaaggtgagcaacaagggcctgcccagcagcatcgagaagaccatcagcaaggccaagggccagcccagggagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaggctgaccgtggacaagagcaggtggcaggagggcaacgtgttcagctgcagcgtgatgcacgaggccctgcacaaccactacacccagaagagcctgagcctgagcctgggcaagatggccctgatcgtgctgggcggcgtggccggcctgctgctgttcatcggcctgggcatcttcttcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 27 - protein, 469 aa, synthetic construct
  REGION 1..469: note = Synthetic
VNCSNMIDEIITHLKQPPLPLLDFNNLNGEDQDILMENNLRRPNLEAFNRAVKSLQNASAIESILKNLLPCLPLATAAPTRHPIHIKDGDWNEFRRELTFYLKTLEESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - DNA, 1407 bp, synthetic construct
  misc_feature 1..1407: note = Synthetic
  source 1..1407: mol_type = other DNA
gtgaactgcagcaacatgatcgacgagatcatcacccacctgaagcagccccccctgcccctgctggacttcaacaacctgaacggcgaggaccaggacatcctgatggagaacaacctgaggaggcccaacctggaggccttcaacagggccgtgaagagcctgcagaacgccagcgccatcgagagcatcctgaagaacctgctgccctgcctgcccctggccaccgccgcccccaccaggcaccccatccacatcaaggacggcgactggaacgagttcaggagggagctgaccttctacctgaagaccctggaggagagcaagtacggccccccctgccccccctgccccgcccccgagttcctgggcggccccagcgtgttcctgttcccccccaagcccaaggacaccctgatgatcagcaggacccccgaggtgacctgcgtggtggtggacgtgagccaggaggaccccgaggtgcagttcaactggtacgtggacggcgtggaggtgcacaacgccaagaccaagcccagggaggagcagttcaacagcacctacagggtggtgagcgtgctgaccgtgctgcaccaggactggctgaacggcaaggagtacaagtgcaaggtgagcaacaagggcctgcccagcagcatcgagaagaccatcagcaaggccaagggccagcccagggagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaggctgaccgtggacaagagcaggtggcaggagggcaacgtgttcagctgcagcgtgatgcacgaggccctgcacaaccactacacccagaagagcctgagcctgagcctgggcaagatggccctgatcgtgctgggcggcgtggccggcctgctgctgttcatcggcctgggcatcttcttcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 29 - protein, 510 aa, synthetic construct
  REGION 1..510: note = Synthetic
VNCSNMIDEIITHLKQPPLPLLDFNNLNGEDQDILMENNLRRPNLEAFNRAVKSLQNASAIESILKNLLPCLPLATAAPTRHPIHIKDGDWNEFRRELTFYLKTLEESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRSKRSRLLHSDYMNMTPRRPGPTRKHQYPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - DNA, 1530 bp, synthetic construct
  misc_feature 1..1530: note = Synthetic
  source 1..1530: mol_type = other DNA
gtgaactgcagcaacatgatcgacgagatcatcacccacctgaagcagccccccctgcccctgctggacttcaacaacctgaacggcgaggaccaggacatcctgatggagaacaacctgaggaggcccaacctggaggccttcaacagggccgtgaagagcctgcagaacgccagcgccatcgagagcatcctgaagaacctgctgccctgcctgcccctggccaccgccgcccccaccaggcaccccatccacatcaaggacggcgactggaacgagttcaggagggagctgaccttctacctgaagaccctggaggagagcaagtacggccccccctgccccccctgccccgcccccgagttcctgggcggccccagcgtgttcctgttcccccccaagcccaaggacaccctgatgatcagcaggacccccgaggtgacctgcgtggtggtggacgtgagccaggaggaccccgaggtgcagttcaactggtacgtggacggcgtggaggtgcacaacgccaagaccaagcccagggaggagcagttcaacagcacctacagggtggtgagcgtgctgaccgtgctgcaccaggactggctgaacggcaaggagtacaagtgcaaggtgagcaacaagggcctgcccagcagcatcgagaagaccatcagcaaggccaagggccagcccagggagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaggctgaccgtggacaagagcaggtggcaggagggcaacgtgttcagctgcagcgtgatgcacgaggccctgcacaaccactacacccagaagagcctgagcctgagcctgggcaagatggccctgatcgtgctgggcggcgtggccggcctgctgctgttcatcggcctgggcatcttcttcaggagcaagaggagcaggctgctgcacagcgactacatgaacatgacccccaggaggcccggccccaccaggaagcaccagtacccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 31 - protein, 511 aa, synthetic construct
  REGION 1..511: note = Synthetic
VNCSNMIDEIITHLKQPPLPLLDFNNLNGEDQDILMENNLRRPNLEAFNRAVKSLQNASAIESILKNLLPCLPLATAAPTRHPIHIKDGDWNEFRRELTFYLKTLEESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - DNA, 1533 bp, synthetic construct
  misc_feature 1..1533: note = Synthetic
  source 1..1533: mol_type = other DNA
gtgaactgcagcaacatgatcgacgagatcatcacccacctgaagcagccccccctgcccctgctggacttcaacaacctgaacggcgaggaccaggacatcctgatggagaacaacctgaggaggcccaacctggaggccttcaacagggccgtgaagagcctgcagaacgccagcgccatcgagagcatcctgaagaacctgctgccctgcctgcccctggccaccgccgcccccaccaggcaccccatccacatcaaggacggcgactggaacgagttcaggagggagctgaccttctacctgaagaccctggaggagagcaagtacggccccccctgccccccctgccccgcccccgagttcctgggcggccccagcgtgttcctgttcccccccaagcccaaggacaccctgatgatcagcaggacccccgaggtgacctgcgtggtggtggacgtgagccaggaggaccccgaggtgcagttcaactggtacgtggacggcgtggaggtgcacaacgccaagaccaagcccagggaggagcagttcaacagcacctacagggtggtgagcgtgctgaccgtgctgcaccaggactggctgaacggcaaggagtacaagtgcaaggtgagcaacaagggcctgcccagcagcatcgagaagaccatcagcaaggccaagggccagcccagggagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaggctgaccgtggacaagagcaggtggcaggagggcaacgtgttcagctgcagcgtgatgcacgaggccctgcacaaccactacacccagaagagcctgagcctgagcctgggcaagatggccctgatcgtgctgggcggcgtggccggcctgctgctgttcatcggcctgggcatcttcttcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 33 - protein, 471 aa, synthetic construct
  REGION 1..471: note = Synthetic
VNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLEESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - DNA, 1413 bp, synthetic construct
  misc_feature 1..1413: note = Synthetic
  source 1..1413: mol_type = other DNA
gtgaactgcagcaacatgatcgacgagatcatcacccacctgaagcagccccccctgcccctgctggacttcaacaacctgaacggcgaggaccaggacatcctgatggagaacaacctgaggaggcccaacctggaggccttcaacagggccgtgaagagcctgcagaacgccagcgccatcgagagcatcctgaagaacctgctgccctgcctgcccctggccaccgccgcccccaccaggcaccccatccacatcaaggacggcgactggaacgagttcaggaggaagctgaccttctacctgaagaccctggaggagagcaagtacggccccccctgccccccctgccccgcccccgagttcctgggcggccccagcgtgttcctgttcccccccaagcccaaggacaccctgatgatcagcaggacccccgaggtgacctgcgtggtggtggacgtgagccaggaggaccccgaggtgcagttcaactggtacgtggacggcgtggaggtgcacaacgccaagaccaagcccagggaggagcagttcaacagcacctacagggtggtgagcgtgctgaccgtgctgcaccaggactggctgaacggcaaggagtacaagtgcaaggtgagcaacaagggcctgcccagcagcatcgagaagaccatcagcaaggccaagggccagcccagggagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaggctgaccgtggacaagagcaggtggcaggagggcaacgtgttcagctgcagcgtgatgcacgaggccctgcacaaccactacacccagaagagcctgagcctgagcctgggcaagatctacatctgggcccccctggccggcacctgcggcgtgctgctgctgagcctggtgatcaccctgtactgcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 35 - protein, 468 aa, synthetic construct
  REGION 1..468: note = Synthetic
VNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLEESKYGPPCPSCPAPEFEGGPSVFLPPKPKDTLMISRTPEVTCVVVDVSQEDPEVQFNWYVDGVEVHQAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - DNA, 1404 bp, synthetic construct
  misc_feature 1..1404: note = Synthetic
  misc_feature 481..483: note = n is a, c, g, or t
  misc_feature 517..519: note = n is a, c, g, or t
  misc_feature 592..594: note = n is a, c, g, or t
  misc_feature 700..702: note = n is a, c, g, or t
  misc_feature 724..726: note = n is a, c, g, or t
  misc_feature 745..747: note = n is a, c, g, or t
  misc_feature 817..819: note = n is a, c, g, or t
  misc_feature 973..975: note = n is a, c, g, or t
  source 1..1404: mol_type = other DNA
gtgaactgcagcaacatgatcgacgagatcatcacccacctgaagcagccccccctgcccctgctggacttcaacaacctgaacggcgaggaccaggacatcctgatggagaacaacctgaggaggcccaacctggaggccttcaacagggccgtgaagagcctgcagaacgccagcgccatcgagagcatcctgaagaacctgctgccctgcctgcccctggccaccgccgcccccaccaggcaccccatccacatcaaggacggcgactggaacgagttcaggaggaagctgaccttctacctgaagaccctggaggagagcaagtacggccccccctgccccagctgccccgcccccgagttcgagggcggccccagcgtgttcctgccccccaagcccaaggacaccctgatgatcagcaggacccccgaggtgacctgcgtggtggtggacgtgagccaggaggaccccgaggtgnnnttcaactggtacgtggacggcgtggaggtgcacnnngccaagaccaagcccagggaggagcagttcaacagcacctacagggtggtgagcgtgctgaccgtgctgcacnnngactggctgaacggcaaggagtacaagtgcaaggtgagcaacaagggcctgcccagcagcatcgagaagaccatcagcaaggccaagggccagcccagggagcccnnngtgtacaccctgccccccagcnnngaggagatgaccaagaacnnngtgagcctgacctgcctggtgaagggcttctaccccagcgacatcgccgtggagtgggagagcaacggcnnncccgagaacaactacaagaccaccccccccgtgctggacagcgacggcagcttcttcctgtacagcaggctgaccgtggacaagagcaggtggcaggagggcaacgtgttcagctgcagcgtgatgcacgaggccctgcacaaccactacaccnnnaagagcctgagcctgagcctgggcaagatggccctgatcgtgctgggcggcgtggccggcctgctgctgttcatcggcctgggcatcttcttcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 37 - protein, 285 aa, synthetic construct
  REGION 1..285: note = Synthetic
VNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLETTTPAPRPPTPAPTIASQPLSLRPEACRPAAGGAVHTRGLDFACDMALIVLGGVAGLLLFIGLGIFFRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - DNA, 855 bp, synthetic construct
  misc_feature 1..855: note = Synthetic
  misc_feature 370..372: note = n is a, c, g, or t
  source 1..855: mol_type = other DNA
gtgaactgcagcaacatgatcgacgagatcatcacccacctgaagcagccccccctgcccctgctggacttcaacaacctgaacggcgaggaccaggacatcctgatggagaacaacctgaggaggcccaacctggaggccttcaacagggccgtgaagagcctgcagaacgccagcgccatcgagagcatcctgaagaacctgctgccctgcctgcccctggccaccgccgcccccaccaggcaccccatccacatcaaggacggcgactggaacgagttcaggaggaagctgaccttctacctgaagaccctggagaccaccacccccgcccccaggccccccacccccgcccccaccatcgccagcnnncccctgagcctgaggcccgaggcctgcaggcccgccgccggcggcgccgtgcacaccaggggcctggacttcgcctgcgacatggccctgatcgtgctgggcggcgtggccggcctgctgctgttcatcggcctgggcatcttcttcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 39 - protein, 469 aa, synthetic construct
  REGION 1..469: note = Synthetic
VNCSNMIDEIITHLKQPPLPLLDFNNLNGEDQDILMENNLRRPNLEAFNRAVKSLQNASAIESILKNLLPCLPLATAAPTRHPIHIKAGDWNEFRRKLTFYLVTLEESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - DNA, 1407 bp, synthetic construct
  misc_feature 1..1407: note = Synthetic
  source 1..1407: mol_type = other DNA
gtgaactgcagcaacatgatcgacgagatcatcacccacctgaagcagccccccctgcccctgctggacttcaacaacctgaacggcgaggaccaggacatcctgatggagaacaacctgaggaggcccaacctggaggccttcaacagggccgtgaagagcctgcagaacgccagcgccatcgagagcatcctgaagaacctgctgccctgcctgcccctggccaccgccgcccccaccaggcaccccatccacatcaaggccggcgactggaacgagttcaggaggaagctgaccttctacctggtgaccctggaggagagcaagtacggccccccctgccccccctgccccgcccccgagttcctgggcggccccagcgtgttcctgttcccccccaagcccaaggacaccctgatgatcagcaggacccccgaggtgacctgcgtggtggtggacgtgagccaggaggaccccgaggtgcagttcaactggtacgtggacggcgtggaggtgcacaacgccaagaccaagcccagggaggagcagttcaacagcacctacagggtggtgagcgtgctgaccgtgctgcaccaggactggctgaacggcaaggagtacaagtgcaaggtgagcaacaagggcctgcccagcagcatcgagaagaccatcagcaaggccaagggccagcccagggagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaggctgaccgtggacaagagcaggtggcaggagggcaacgtgttcagctgcagcgtgatgcacgaggccctgcacaaccactacacccagaagagcctgagcctgagcctgggcaagatggccctgatcgtgctgggcggcgtggccggcctgctgctgttcatcggcctgggcatcttcttcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 41 - protein, 511 aa, synthetic construct
  REGION 1..511: note = Synthetic
VNCSNMIDEIITHLKQPPLPLLDFNNLNGEDQDILMENNLRRPNLEAFNRAVKSLQNASAIESILKNLLPCLPLATAAPTRHPIHIKAGDWNEFRRKLTFYLVTLEESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - DNA, 1533 bp, synthetic construct
  misc_feature 1..1533: note = Synthetic
  source 1..1533: mol_type = other DNA
gtgaactgcagcaacatgatcgacgagatcatcacccacctgaagcagccccccctgcccctgctggacttcaacaacctgaacggcgaggaccaggacatcctgatggagaacaacctgaggaggcccaacctggaggccttcaacagggccgtgaagagcctgcagaacgccagcgccatcgagagcatcctgaagaacctgctgccctgcctgcccctggccaccgccgcccccaccaggcaccccatccacatcaaggccggcgactggaacgagttcaggaggaagctgaccttctacctggtgaccctggaggagagcaagtacggccccccctgccccccctgccccgcccccgagttcctgggcggccccagcgtgttcctgttcccccccaagcccaaggacaccctgatgatcagcaggacccccgaggtgacctgcgtggtggtggacgtgagccaggaggaccccgaggtgcagttcaactggtacgtggacggcgtggaggtgcacaacgccaagaccaagcccagggaggagcagttcaacagcacctacagggtggtgagcgtgctgaccgtgctgcaccaggactggctgaacggcaaggagtacaagtgcaaggtgagcaacaagggcctgcccagcagcatcgagaagaccatcagcaaggccaagggccagcccagggagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaggctgaccgtggacaagagcaggtggcaggagggcaacgtgttcagctgcagcgtgatgcacgaggccctgcacaaccactacacccagaagagcctgagcctgagcctgggcaagatggccctgatcgtgctgggcggcgtggccggcctgctgctgttcatcggcctgggcatcttcttcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 43 - protein, 510 aa, synthetic construct
  REGION 1..510: note = Synthetic
VNCSNMIDEIITHLKQPPLPLLDFNNLNGEDQDILMENNLRRPNLEAFNRAVKSLQNASAIESILKNLLPCLPLATAAPTRHPIHIKAGDWNEFRRKLTFYLVTLEESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRSKRSRLLHSDYMNMTPRRPGPTRKHQYPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - DNA, 1530 bp, synthetic construct
  misc_feature 1..1530: note = Synthetic
  source 1..1530: mol_type = other DNA
gtgaactgcagcaacatgatcgacgagatcatcacccacctgaagcagccccccctgcccctgctggacttcaacaacctgaacggcgaggaccaggacatcctgatggagaacaacctgaggaggcccaacctggaggccttcaacagggccgtgaagagcctgcagaacgccagcgccatcgagagcatcctgaagaacctgctgccctgcctgcccctggccaccgccgcccccaccaggcaccccatccacatcaaggccggcgactggaacgagttcaggaggaagctgaccttctacctggtgaccctggaggagagcaagtacggccccccctgccccccctgccccgcccccgagttcctgggcggccccagcgtgttcctgttcccccccaagcccaaggacaccctgatgatcagcaggacccccgaggtgacctgcgtggtggtggacgtgagccaggaggaccccgaggtgcagttcaactggtacgtggacggcgtggaggtgcacaacgccaagaccaagcccagggaggagcagttcaacagcacctacagggtggtgagcgtgctgaccgtgctgcaccaggactggctgaacggcaaggagtacaagtgcaaggtgagcaacaagggcctgcccagcagcatcgagaagaccatcagcaaggccaagggccagcccagggagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaggctgaccgtggacaagagcaggtggcaggagggcaacgtgttcagctgcagcgtgatgcacgaggccctgcacaaccactacacccagaagagcctgagcctgagcctgggcaagatggccctgatcgtgctgggcggcgtggccggcctgctgctgttcatcggcctgggcatcttcttcaggagcaagaggagcaggctgctgcacagcgactacatgaacatgacccccaggaggcccggccccaccaggaagcaccagtacccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 45 - protein, 471 aa, synthetic construct
  REGION 1..471: note = Synthetic
VNCSNMIDEIITHLKQPPLPLLDFNNLNGEDQDILMENNLRRPNLEAFNRAVKSLQNASAIESILKNLLPCLPLATAAPTRHPIHIKAGDWNEFRRKLTFYLVTLEESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - DNA, 1413 bp, synthetic construct
  misc_feature 1..1413: note = Synthetic
  source 1..1413: mol_type = other DNA
gtgaactgcagcaacatgatcgacgagatcatcacccacctgaagcagccccccctgcccctgctggacttcaacaacctgaacggcgaggaccaggacatcctgatggagaacaacctgaggaggcccaacctggaggccttcaacagggccgtgaagagcctgcagaacgccagcgccatcgagagcatcctgaagaacctgctgccctgcctgcccctggccaccgccgcccccaccaggcaccccatccacatcaaggccggcgactggaacgagttcaggaggaagctgaccttctacctggtgaccctggaggagagcaagtacggccccccctgccccccctgccccgcccccgagttcctgggcggccccagcgtgttcctgttcccccccaagcccaaggacaccctgatgatcagcaggacccccgaggtgacctgcgtggtggtggacgtgagccaggaggaccccgaggtgcagttcaactggtacgtggacggcgtggaggtgcacaacgccaagaccaagcccagggaggagcagttcaacagcacctacagggtggtgagcgtgctgaccgtgctgcaccaggactggctgaacggcaaggagtacaagtgcaaggtgagcaacaagggcctgcccagcagcatcgagaagaccatcagcaaggccaagggccagcccagggagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaggctgaccgtggacaagagcaggtggcaggagggcaacgtgttcagctgcagcgtgatgcacgaggccctgcacaaccactacacccagaagagcctgagcctgagcctgggcaagatctacatctgggcccccctggccggcacctgcggcgtgctgctgctgagcctggtgatcaccctgtactgcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 47 - protein, 468 aa, synthetic construct
  REGION 1..468: note = Synthetic
VNCSNMIDEIITHLKQPPLPLLDFNNLNGEDQDILMENNLRRPNLEAFNRAVKSLQNASAIESILKNLLPCLPLATAAPTRHPIHIKAGDWNEFRRKLTFYLVTLEESKYGPPCPSCPAPEFEGGPSVFLPPKPKDTLMISRTPEVTCVVVDVSQEDPEVQFNWYVDGVEVHQAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - DNA, 1404 bp, synthetic construct
  misc_feature 1..1404: note = Synthetic
  misc_feature 481..483: note = n is a, c, g, or t
  misc_feature 517..519: note = n is a, c, g, or t
  misc_feature 592..594: note = n is a, c, g, or t
  misc_feature 700..702: note = n is a, c, g, or t
  misc_feature 724..726: note = n is a, c, g, or t
  misc_feature 745..747: note = n is a, c, g, or t
  misc_feature 817..819: note = n is a, c, g, or t
  misc_feature 973..975: note = n is a, c, g, or t
  source 1..1404: mol_type = other DNA
gtgaactgcagcaacatgatcgacgagatcatcacccacctgaagcagcctcctctgcctctgctggacttcaacaacctgaacggcgaggaccaggacatcctgatggagaacaacctgaggaggcctaacctggaggccttcaacagggccgtgaagagcctgcagaacgccagcgccatcgagagcatcctgaagaacctgctgccttgcctgcctctggccaccgccgcccctaccaggcaccctatccacatcaaggccggcgactggaacgagttcaggaggaagctgaccttctacctggtgaccctggaggagagcaagtacggccctccttgccctagctgccctgcccctgagttcgagggcggccctagcgtgttcctgcctcctaagcctaaggacaccctgatgatcagcaggacccctgaggtgacctgcgtggtggtggacgtgagccaggaggaccctgaggtgnnnttcaactggtacgtggacggcgtggaggtgcacnnngccaagaccaagcctagggaggagcagttcaacagcacctacagggtggtgagcgtgctgaccgtgctgcacnnngactggctgaacggcaaggagtacaagtgcaaggtgagcaacaagggcctgcctagcagcatcgagaagaccatcagcaaggccaagggccagcctagggagcctnnngtgtacaccctgcctcctagcnnngaggagatgaccaagaacnnngtgagcctgacctgcctggtgaagggcttctaccctagcgacatcgccgtggagtgggagagcaacggcnnncctgagaacaactacaagaccacccctcctgtgctggacagcgacggcagcttcttcctgtacagcaggctgaccgtggacaagagcaggtggcaggagggcaacgtgttcagctgcagcgtgatgcacgaggccctgcacaaccactacaccnnnaagagcctgagcctgagcctgggcaagatggccctgatcgtgctgggcggcgtggccggcctgctgctgttcatcggcctgggcatcttcttcagggtgaagttcagcaggagcgccgacgcccctgcctaccagcagggccagaaccagctgtacaacgagctgaacctgggcaggagggaggagtacgacgtgctggacaagaggaggggcagggaccctgagatgggcggcaagcctaggaggaagaaccctcaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgcctcctagg

SEQ ID NO: 49 - protein, 285 aa, synthetic construct
  REGION 1..285: note = Synthetic
VNCSNMIDEIITHLKQPPLPLLDFNNLNGEDQDILMENNLRRPNLEAFNRAVKSLQNASAIESILKNLLPCLPLATAAPTRHPIHIKAGDWNEFRRKLTFYLVTLETTTPAPRPPTPAPTIASQPLSLRPEACRPAAGGAVHTRGLDFACDMALIVLGGVAGLLLFIGLGIFFRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - DNA, 918 bp, synthetic construct
  misc_feature 1..918: note = Synthetic
  misc_feature 433..435: note = n is a, c, g, or t
  source 1..918: mol_type = other DNA
atgctgctgctggtgaccagcctgctgctgtgcgagctgccccaccccgccttcctgctgatcgtgaactgcagcaacatgatcgacgagatcatcacccacctgaagcagccccccctgcccctgctggacttcaacaacctgaacggcgaggaccaggacatcctgatggagaacaacctgaggaggcccaacctggaggccttcaacagggccgtgaagagcctgcagaacgccagcgccatcgagagcatcctgaagaacctgctgccctgcctgcccctggccaccgccgcccccaccaggcaccccatccacatcaaggccggcgactggaacgagttcaggaggaagctgaccttctacctggtgaccctggagaccaccacccccgcccccaggccccccacccccgcccccaccatcgccagcnnncccctgagcctgaggcccgaggcctgcaggcccgccgccggcggcgccgtgcacaccaggggcctggacttcgcctgcgacatggccctgatcgtgctgggcggcgtggccggcctgctgctgttcatcggcctgggcatcttcttcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 51 - protein, 479 aa, synthetic construct
  REGION 1..479: note = Synthetic
VNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLWTLEGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - DNA, 1437 bp, synthetic construct
  misc_feature 1..1437: note = Synthetic
  source 1..1437: mol_type = other DNA
gtgaactgcagcaacatgatcgacgagatcatcacccacctgaagcagccccccctgcccctgctggacttcaacaacctgaacggcgaggaccaggacatcctgatggagaacaacctgagaagacccaacctggaggccttcaacagagccgtgaagagcctgcagaacgccagcgccatcgagagcatcctgaagaacctgctgccctgcctgcccctggccaccgccgcccccaccagacaccccatccacatcaaggacggcgactggaacgagttcagaagaaagctgaccttctacctgtggaccctggagggcggcggcggcagcggcggcggcggcagcgagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagatggccctgatcgtgctgggcggcgtggccggcctgctgctgttcatcggcctgggcatcttcttcagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccaga

SEQ ID NO: 53 - protein, 293 aa, synthetic construct
  REGION 1..293: note = Synthetic
MALPVTALLLPLALLLHAARPMADYKDIVMTQSHKFMSTSVGDRVNITCKASQNVDSAVAWYQQKPGQSPKALIYSASYRYSGVPDRFTGRGSGTDFTLTISSVQAEDLAVYYCQQYYSTPWTFGGGTKLEIKRGGGGSGGGGSGGGGSEVKLVESGGGLVQPGGSLSLSCAASGFTFTDYYMSWVRQPPGKALEWLALIRSKADGYTTEYSASVKGRFTLSRDDSQSILYLQMNALRPEDSATYYCARDAAYYSYYSPEGAMDYWGQGTSVTVSSTTTPAPRPPTPAPTIAS

SEQ ID NO: 54 - DNA, 5197 bp, synthetic construct
  misc_feature 1..5197: note = Synthetic
  source 1..5197: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagaattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgcgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgtctagagctagcggatccccggaattcgacgccaccatgctgctgctggtgaccagcctgctgctgtgcgagctgccccaccccgccttcctgctgatcgtgaactgcagcaacatgatcgacgagatcatcacccacctgaagcagccccccctgcccctgctggacttcaacaacctgaacggcgaggaccaggacatcctgatggagaacaacctgaggaggcccaacctggaggccttcaacagggccgtgaagagcctgcagaacgccagcgccatcgagagcatcctgaagaacctgctgccctgcctgcccctggccaccgccgcccccaccaggcaccccatccacatcaaggacggcgactggaacgagttcaggaggaagctgaccttctacctgaagaccctggaggagagcaagtacggccccccctgccccccctgccccgcccccgagttcctgggcggccccagcgtgttcctgttcccccccaagcccaaggacaccctgatgatcagcaggacccccgaggtgacctgcgtggtggtggacgtgagccaggaggaccccgaggtgcagttcaactggtacgtggacggcgtggaggtgcacaacgccaagaccaagcccagggaggagcagttcaacagcacctacagggtggtgagcgtgctgaccgtgctgcaccaggactggctgaacggcaaggagtacaagtgcaaggtgagcaacaagggcctgcccagcagcatcgagaagaccatcagcaaggccaagggccagcccagggagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaggctgaccgtggacaagagcaggtggcaggagggcaacgtgttcagctgcagcgtgatgcacgaggccctgcacaaccactacacccagaagagcctgagcctgagcctgggcaagatggccctgatcgtgctgggcggcgtggccggcctgctgctgttcatcggcctgggcatcttcttcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagggagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaattaattaaccaattgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 55 - DNA, 5323 bp, synthetic construct
  misc_feature 1..5323: note = Synthetic
  source 1..5323: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagaattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgcgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgtctagagctagcggatccccggaattcgacgccaccatgctgctgctggtgaccagcctgctgctgtgcgagctgccccaccccgccttcctgctgatcgtgaactgcagcaacatgatcgacgagatcatcacccacctgaagcagccccccctgcccctgctggacttcaacaacctgaacggcgaggaccaggacatcctgatggagaacaacctgaggaggcccaacctggaggccttcaacagggccgtgaagagcctgcagaacgccagcgccatcgagagcatcctgaagaacctgctgccctgcctgcccctggccaccgccgcccccaccaggcaccccatccacatcaaggacggcgactggaacgagttcaggaggaagctgaccttctacctgaagaccctggaggagagcaagtacggccccccctgccccccctgccccgcccccgagttcctgggcggccccagcgtgttcctgttcccccccaagcccaaggacaccctgatgatcagcaggacccccgaggtgacctgcgtggtggtggacgtgagccaggaggaccccgaggtgcagttcaactggtacgtggacggcgtggaggtgcacaacgccaagaccaagcccagggaggagcagttcaacagcacctacagggtggtgagcgtgctgaccgtgctgcaccaggactggctgaacggcaaggagtacaagtgcaaggtgagcaacaagggcctgcccagcagcatcgagaagaccatcagcaaggccaagggccagcccagggagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaggctgaccgtggacaagagcaggtggcaggagggcaacgtgttcagctgcagcgtgatgcacgaggccctgcacaaccactacacccagaagagcctgagcctgagcctgggcaagatggccctgatcgtgctgggcggcgtggccggcctgctgctgttcatcggcctgggcatcttcttcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagggagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaattaattaaccaattgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 56 - skipped

SEQ ID NO: 57 - protein, 16 aa, synthetic construct
  REGION 1..16: note = Synthetic
  VARIANT 4: note = May also be I
  VARIANT 11..12: note = May be absent
  VARIANT 16: note = May also be I
YXXLXXXXXXXXYXXL

(19) United States
(12) Patent Application Publication
(10) Pub. No.: US 2025/0082755 A1
(43) Pub. Date: Mar. 13, 2025

(54) CHIMERIC ANTIGEN RECEPTORS FOR TREATMENT OF CANCER

(71) Applicant: VOR BIOPHARMA INC., Cambridge, MA (US)

(72) Inventors: Rebecca Moeller, Cambridge, MA (US); Tirtha Chakraborty, Cambridge, MA (US); Julian Scherer, Cambridge, MA (US)

(73) Assignee: VOR BIOPHARMA INC., Cambrige, MA (US)

(21) Appl. No.: 18/292,499

(22) PCT Filed: Jul. 29, 2022
(86) PCT No.: PCT/US2022/074333

§ 371 Date: Jan. 26, 2024

Related U.S. Application Data
(60) Provisional application No. 63/278,019, filed on Nov. 10, 2021.
(60) Provisional application No. 63/227,192, filed on Jul. 29, 2021.

(51) Int. Cl. A61K 39/00

(52) U.S. Cl. CPC A61K 40/4217; A61K 40/11; A61K 40/31; A61K 40/4234

(57) ABSTRACT

Provided herein are chimeric antigen receptors (CARs) with binding specificity for CD123. Nucleic acids, expression vectors, host cells, populations of cells, and pharmaceutical compositions relating to the CARs are also disclosed, and methods including the treatment of a hematopoietic malignancy or premalignancy characterized by the expression of CD123, e.g., leukemias such as acute myeloid leukemia (AML) and myelodysplastic syndrome (MDS).

### RELATED APPLICATIONS

This application claims the benefit under 35 U.S.C. 119 (e) of U.S. Provisional Application No. 63/227,192 filed Jul. 29, 2021 and U.S. Provisional Application No. 63/278,019 filed Nov. 10, 2021, each of which are incorporated by reference herein in its entirety.

### REFERENCE TO AN ELECTRONIC SEQUENCE LISTING

The contents of the electronic sequence listing (V029170032WO00-SEQ-CEW.xml; Size: 117,840 bytes; and Date of Creation: Jul. 29, 2022) is herein incorporated by reference in its entirety.

### BACKGROUND

Acute myelogenous leukemia (AML) is a highly aggressive acute leukemia. Current treatment regimens include intensive cycles of multi-agent chemotherapy, and frequently are performed with allogeneic donor stem cell transplantation to achieve cure, but many AML patients do not achieve long-term remission. New therapeutic strategies are needed to increase remission rates, decrease relapse, and to improve overall survival.

### SUMMARY

Aspects of the present disclosure provide chimeric antigen receptors (CARs), comprising: (a) an interleukin-3 (IL-3) molecule or a CD123-binding fragment thereof; (b) optionally, a linker region, (c) optionally, a hinge region, (d) a transmembrane region, (e) optionally, at least one costimulatory signaling domain, and (f) a signaling domain.

In some embodiments, the IL-3 molecule, or CD123-binding fragment thereof, comprises a substitution mutation at position K110, D101, K116, or a combination thereof. In some embodiments, the IL-3 molecule, or CD123-binding fragment thereof, comprises a D101 mutation, a K116V mutation, a K116W mutation, or a combination thereof. In some embodiments, the IL-3 molecule, or CD123-binding fragment thereof, comprises a D101A mutation and a K116V mutation.

In some embodiments, the signaling domain is a CD3 zeta (CD3ζ) signaling domain.

In some embodiments, the CAR comprises at least one costimulatory signaling domain that is a CD28, 4-1BB, and/or OX-40 costimulatory signaling domain. In some embodiments, the CAR comprises a CD28 costimulatory signaling domain. In some embodiments, the CAR comprises a 4-1BB costimulatory signaling domain. In some embodiments, the CAR comprises an OX-40 costimulatory signaling domain. In some embodiments, the CAR does not comprise a costimulatory signaling domain.

In some embodiments, the transmembrane region is a human CD4 transmembrane region. In some embodiments, the transmembrane region is a human CD8 transmembrane region. In some embodiments, the transmembrane region is a human CD28 transmembrane region.

In some embodiments, the CAR comprises a hinge region that is a human immunoglobulin (Ig) subclass G4 (IgG4) fragment crystallizable (Fc) hinge region. In some embodiments, the CAR comprises a linker region that is a human immunoglobulin (Ig) subclass G4 (IgG4) fragment crystallizable (Fc) linker region. In some embodiments, the human IgG4 Fc linker region comprises a substitution mutation at position corresponding to L235, N297, or a combination thereof. In some embodiments, the human IgG4 Fc linker region comprises a L235E mutation, a N297Q mutation, or a combination thereof.

In some embodiments, the CAR comprises, from N-terminus to C-terminus: (a) the interleukin-3 (IL-3) molecule or a CD123-binding fragment thereof; (b) the linker region and/or the hinge region, (c) the transmembrane region, and (d) the signaling domain.

In some embodiments, the CAR comprises an amino acid sequence having at least 95% sequence identity to any one of SEQ ID NOs: 21, 27, 33, 35, 37, 39, 45, 47, 49, or 51. In some embodiments, the CAR comprises an amino acid sequence of any one of SEQ ID NOs: 21, 33, 35, 37, 39, 41, 45, 47, 49, or 51. In some embodiments, the CAR consists of an amino acid sequence of any one of SEQ ID NOs: 21, 27, 33, 35, 37, 39, 45, 47, 49, or 51.

In some embodiments, the CAR comprises, from N-terminus to C-terminus: (a) the interleukin-3 (IL-3) molecule or a CD123-binding fragment thereof; (b) the linker region and/or the hinge region, (c) the transmembrane region, and (d) the one or more co-stimulatory signaling domains, and (c) the signaling domain.

In some embodiments, the CAR comprises an amino acid sequence having at least 95% sequence identity to any one of SEQ ID NOs: 23, 25, 29, 31, 41, or 43. In some embodiments, the CAR comprises an amino acid sequence of any one of SEQ ID NOs: 23, 25, 29, 31, 41, or 43. In some embodiments, the CAR consists of an amino acid sequence of any one of SEQ ID NOs: 23, 25, 29, 31, 41, or 43.

In some embodiments, the CAR further comprises a signal peptide/signal sequence.

Aspects of the present disclosure provide nucleic acid constructs encoding any one of the CARs provided herein. In some embodiments, the nucleic acid construct further comprises a promoter sequence. In some embodiments, the nucleic acid is RNA. In some embodiments, the nucleic acid is DNA. In some embodiments, the nucleic acid construct comprises a sequence having at least 95% sequence identity to any one of SEQ ID NOs: 22, 24, 26, 28, 30, 32, 34, 36, 38, 40, 42, 44, 46, 38, 50, or 52. In some embodiments, the nucleic acid construct comprises an amino acid sequence of any one of SEQ ID NOs: 22, 24, 26, 28, 30, 32, 34, 36, 38, 40, 42, 44, 46, 38, 50, or 52. In some embodiments, the nucleic acid construct consists of an amino acid sequence of any one of SEQ ID NOs: 22, 24, 26, 28, 30, 32, 34, 36, 38, 40, 42, 44, 46, 38, 50, or 52.

Aspects of the present disclosure provide vectors comprising any one of the nucleic acid constructs provided herein. In some embodiments, the vector is a DNA vector, an RNA vector, a plasmid, a lentivirus vector, an adenoviral vector, or a retrovirus vector.

Aspects of the present disclosure provide cells comprising any of the nucleic acid constructs provided herein.

Aspects of the present disclosure provide cells expressing any one of the CARs provided herein. In some embodiments, the cell is an immune effector cell. In some embodiments, the cell is a T-lymphocyte. In some embodiments, the cell is a NK cell.

Aspects of the present disclosure provide pharmaceutical compositions comprising any one of the cells provided herein.

Aspects of the present disclosure provide methods comprising administering any one of the CARs provided herein, any one of the nucleic acid constructs provided herein, any one of the vectors provided herein, any one of the cells, or any of the pharmaceutical compositions provided herein to a subject in need thereof.

In some embodiments, the subject has or has been diagnosed with a hematopoietic malignancy or pre-malignancy characterized by the expression of CD123 on malignant cells or pre-malignant cells. In some embodiments, the hematopoietic malignancy is acute myeloid leukemia (AML). In some embodiments, the hematopoictic malignancy is myelodysplastic syndrome (MDS).

In some embodiments, any one of the methods provided herein further comprises administering a population of hematopoietic cells, wherein the hematopoietic cells are genetically-engineered such that the gene encoding CD123 is engineered to reduce or eliminate the expression of CD123.

Aspects of the present disclosure provide methods comprising introducing into a cell any one of the nucleic acid constructs provided herein or any one of the vectors provided herein. In some embodiments, the cell is obtained from, or derived from, a subject (e.g., a healthy subject or a subject in need of treatment) prior to introducing the nucleic acid construct or vector. In some embodiments, the subject has or has been diagnosed with a hematopoictic malignancy or pre-malignancy characterized by the expression of CD123 on malignant cells or pre-malignant cells.

In some embodiments, the cell is an immune effector cell. In some embodiments, the cell is a T-lymphocyte. In some embodiments, the cell is a NK cell. In some embodiments, the T-lymphocyte or NK cell is activated and/or expanded ex vivo. In some embodiments, the nucleic acid or vector is introduced into the cell by lentiviral transduction, retroviral transduction, adeno-associated viral transduction, DNA electroporation, RNA electroporation, or transposon electroporation.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIGS. 1A and 1B show diagrams depicting CD123 expression in leukemia. FIG. 1A shows low expression on normal hematopoietic stem cells (HSC, CD123_{low}) and high expression on leukemic stem cells (LSCs, CD123_{high}), which have the capacity for self-renewal. These cells may lead to minimal residue disease and disease relapse. Leukemic progenitor cells and leukemic blasts both exhibit a medium level of CD123 expression (CD123_{med}). FIG. 1B shows that high expression levels of CD123 in patients with AML correlate with significantly reduced probability of survival over time as compared to low/medium expression levels of CD123.

FIGS. 2A and 2B show diagrams depicting the predicted mechanism of CD123: IL-3 binding and the structure of exemplary IL3-zetakine constructs. FIG. 2A shows IL3Rα (CD123) binding to its ligand, IL-3. IL3Rβ (CSF2RB) joins the complex to initiate Janus kinase (JAK)/signal transducer and activator of transcription pathway signaling. FIG. 2B shows the dimerized structure of an exemplary IL3-zetakine construct, comprising extracellular human IL3 (huIL3), an Fc gamma 4 (γ₄Fc) hinge region, CD4 or CD8 transmembrane domain, and a CD3ζ intracellular signaling domain.

FIGS. 3A and 3B show flow cytometry plots of CD123-specific induction of CD69 expression in cells expressing a IL3-zetakine. FIG. 3A shows an increase in CD69 activation in TIB-153™ cells (a T cell line) expressing a IL3-zetakine (IL3-WT-nocostim (SEQ ID NO: 21)) in the presence of CD123-expressing MOLM13 cells (MOLM13) as compared to TIB-153™ cells expressing a IL3-zetakine alone and to TIB-153™ cells expressing a IL3-zetakine in the presence of MOLM13 cells deficient in CD123 (MOLM13-CD123KO). FIG. 3B shows CD69 activation in TIB-153™ cells expressing a IL3-zeta comprising a 4-1BB costimulatory signaling domain (WT-41BBcostim (SEQ ID NO: 25)) in the presence of CD123-expressing MOLM13 cells (MOLM13) as compared to TIB-153TM cells expressing the IL3-zetakine alone or TIB-153™ cells expressing the IL3-zetakine in the presence of MOLM13 cells deficient in CD123 (MOLM13-CD123KO). Y-axis: count; x-axis: CD69 expression.

FIG. 4 shows the percent CD69+ cells activation in untransduced TIB-153TM cells or IL3-zetakine-expressing TIB-153™ cells (Z-WT-No costim (SEQ ID NO: 21)) or IL3-zetakine comprising a 4-1BB costimulatory signaling domain (Z-WT-41BBcostim (SEQ ID NO: 25)) in the presence of MOLM13 cells (MOLM13-WT) as compared the TIB-153TM cells expressing the IL3-zetakines alone or in the presence of MOLM13 cells deficient in CD123 (MOLM13-CD123KO).

FIG. 5 shows the percentage of target cells (MOLM13 or MOLM13-CD123KO cells) alive following 24-hours of co-culturing with TIB-153™ cells. TIB-153TM cells expressing an anti-CD123 CAR and TIB-153™ cells expressing a IL3-zetakine (Z-WT-No costim (SEQ ID NO: 21)) resulted in death of MOLM13 cells (MOLM13-WT (CD123_{high})) as compared to MOLM13 cells deficient in CD123 (MOLM13-CD123KO). Mock cells correspond to TIB-153™ cells that are mock transduced and do not express a CAR or IL3-zetakine.

FIGS. 6A and 6B show flow cytometry plots presenting expression of exemplary IL3-zetakines in TIB-153™ Jurkat cells. FIG. 6A shows expression of the indicated exemplary IL3-zetakines as detected with an anti-IL3 antibody (αIL3). FIG. 6B shows expression of the indicated exemplary IL3-zetakines detected with a biotinylated recombinant CD123 protein bound to fluorophore-tagged streptavidin. The panels show, from left to right, untransduced cells (UTD), IL3-WT-nocostim (SEQ ID NO: 21), IL3-WTCD8TM (SEQ ID NO: 33), and IL3-K116W (SEQ ID NO: 51). The percentage of positive cells is indicated in each panel.

FIG. 7 shows flow cytometry plots presenting expression of the indicated exemplary IL3-zetakines in primary CD3+ T cells as detected with an anti-IL3 antibody (αIL3). The panels show, from left to right, untransduced cells (UTD), IL3-WT-nocostim (SEQ ID NO: 21), IL3-WTCD8TM (SEQ ID NO: 33), and IL3-K116W (SEQ ID NO: 51). The percentage of positive cells is indicated in each panel.

FIGS. 8A-8D show flow cytometry plots of CD123-specific activation of TIB-153™ cells transduced with exemplary IL3-zetakines as measured by CD69 expression. FIG. 8A shows CD69 expression by the TIB-153™ cells cocultured with or without CD123-expressing target cells for 24 hours at an effector to target cell ratio of 1:1. From top to bottom, the rows in each of the panels refer to no target cells, +HL60-WT cells (CD123ₙᵤₗₗ), +MOLM13-CD123_{low}, and +MOLM13-WT (CD123_{high}). FIG. 8B shows expression of CD69 (x-axis) of TIB-153™ cells cocultured with MOLM13-WT target cells (CD123_{high}) as a function of zetakine expression as detected by an anti-IL3 antibody (αIL3, y-axis). FIG. 8C shows expression of CD69 (x-axis) by TIB-153™ cells cocultured with MOLM13 target cells having low CD123 expression (MOLM13-CD123_{low}) as a function of zetakine expression as detected by an anti-IL3 antibody (αIL3, y-axis). FIG. 8D shows a bar graph representation of the flow cytometry data of CD69 expression by the TIB-153™ cells in co-culture with MOLM13-WT (CD123_{high}), MOLM13-CD123_{low}, or HL60WT (CD123ₙᵤₗₗ) target cells or in the absence of target cells. N=3 for all samples. *** refers to p<0.001, one-tailed student's T test. For FIGS. 8A-8C, the panels show, from left to right, untransduced cells (UTD), IL3-WT-nocostim (SEQ ID NO: 21), IL3-WTCD8TM (SEQ ID NO: 33), and IL3-K116W (SEQ ID NO: 51). The percentage of positive cells is indicated in each quadrant of each panel in FIGS. 8B and 8C.

FIG. 9A-9D show flow cytometry plots of CD123-specific activation of primary T cells transduced with exemplary IL3-zetakines or an anti-CD123 chimeric antigen receptor (CD123 CAR) as measured by CD25 expression. FIG. 9A shows CD25 expression by the primary CD3+ T cells co-cultured with or without CD123-expressing targeting cells for 24 hours at an effector to target cell ratio of 1:1. From top to bottom, the rows in each of the panels refer to no target cells, +MOLM13-CD123_{low}, and +MOLM13-WT (CD123_{high}). FIG. 9B shows expression of CD25 (x-axis) and CD69 (y-axis) by CD3+ T cells co-cultured with MOLM13-WT target cells (CD123_{high}). FIG. 9C shows expression of CD25 (x-axis) and CD69 (y-axis) by CD3+ T cells cocultured (MOLM13-CD123_{low}) target cells. FIG. 9D shows a bar graph representation of the flow cytometry data of CD25 expression by the CD3+ cells in co-culture with MOLM13WT or MOLM13-CD123_{low} target cells, or absence of target cells. N=3 for all samples. *** refers to p<0.001. ** refers to p<0.01, and * refers to <0.05, one-tailed student's T test. For FIGS. 9A-9C, the panels show, from left to right, untransduced cells (UTD), anti-CD123 CAR, IL3-WT-nocostim (SEQ ID NO: 21), IL3-WTCD8TM (SEQ ID NO: 33), and IL3-K116W (SEQ ID NO: 51). The percentage of positive cells is indicated in each quadrant of each panel in FIGS. 8B and 8C.

FIG. 10 presents graphs showing CD123-specific cytotoxicity induced by primary T cells transduced with exemplary IL3-zetakine constructs or anti-CD123 CARs. Primary human PBMCs were mock transduced (untransduced) or subjected to lentiviral transduction with IL3-WT-nocostim (SEQ ID NO: 21), IL3-WTCD8TM (SEQ ID NO: 33), IL3-K116W (SEQ ID NO: 51), or transduced with a positive control, anti-CD123 CAR construct (CD123 CAR). The cells were cocultured with MOLM13-WT (CD123_{high}) target cells (left panel), MOLM13-CD123_{low} target cells (middle panel), or HL60-WT (CD123null) target cells (right panel) for 24 hours at an effector to target cell ratio of 1:1. Target cell viability was assessed by flow cytometry: alive cells (viability and annexin V-negative); apoptotic cells (viability negative and annexin V-positive); and dead cells/debris (viability negative and annexin V-positive). For each column, the sections refer, from top to bottom, to dead/debris, apoptotic, and alive. N=3 for all samples.

FIGS. 11A and 11B show a constructs of an exemplary IL-3 zetakine construct. Expression of the IL-3 zetakine (IL3-WT-nocostim; SEQ ID NO: 21) is under control of a spleen focus-forming virus (SFFV) long terminal repeat (LTR) promoter (SFFV promoter) (SEQ ID NO: 20). FIG. 11A shows a plasmid map. A puromycin-N-acetyltransferase gene (PuroR) was used as a selection marker. FIG. 11B shows a lentiviral vector map.

FIGS. 12A and 12B show constructs of an exemplary IL-3 zetakine construct. Expression of the IL-3 zetakine (IL3-WT-41BBcostim; SEQ ID NO: 25) is under control of a SFFV promoter (SEQ ID NO: 20). FIG. 12A shows a plasmid map. A puromycin-N-acetyltransferase gene (PuroR) was used as a selection marker. FIG. 12B shows a lentiviral vector map.

FIG. 13 shows T cells expressing the indicated exemplary IL-3 zetakines demonstrate in vitro cytotoxicity of CD123-expressing target cells. Donor primary T cells expressing the indicated IL-3 zetakines were co-cultured for 24 hours at an effector to target cell ratio ratio of 1:1 (target cells: MOLM13-Wt, MOLM13-CD123_{low}, and HL60-WT). The y-axis shows the change (delta) in target cell lysis relative to target cells alone. The primary donor T cells were to untransduced (UTD) or transduced with IL3-WT-nocostim (SEQ ID NO: 21), IL3-WT-nocostim-CD8TM (SEQ ID NO: 33), and IL3-K116W (SEQ ID NO: 51). The effector to target cell ratio was normalized based on the transduction efficiency (TE), which is indicated as the percentage below the zetakines on the x-axis.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

The articles “a” and “an” are used herein to refer to one or to more than one (i.e., to at least one) of the grammatical object of the article. By way of example, “an element” means one element or more than one element.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Agent: As used herein, the term “agent” (or “biological agent” or “therapeutic agent”), refers to a molecule that may be expressed, released, secreted or delivered to a target by a modified cell (e.g., an immune cell comprising a chimeric antigen receptor) described herein. An agent includes, but is not limited to, a nucleic acid, an antibiotic, an anti-inflammatory agent, an antibody or fragments thereof, a chimeric antigen receptor, an antibody agent or fragments thereof, a growth factor, a cytokine, an enzyme, a protein (e.g., an RNAse inhibitor), a peptide, a fusion protein, a synthetic molecule, an organic molecule (e.g., a small molecule), a carbohydrate, a lipid, a hormone, a microsome, a derivative or a variation thereof, and any combinations thereof. An agent may bind any cell moiety, such as a receptor, an antigenic determinant, or other binding site present on a target or target cell. An agent may diffuse or be transported into a cell, where it may act intracellularly.

Antigen: As used herein, the term “antigen” or “Ag” refers to a molecule that is capable of provoking an immune response. This immune response may involve either antibody production, the activation of specific immunologically-competent cells, or both. A skilled artisan will understand that any macromolecule, including virtually all proteins or peptides, can serve as an antigen. Furthermore, antigens can be derived from recombinant or genomic DNA. A skilled artisan will understand that any DNA that comprises a nucleotide sequence or a partial nucleotide sequence encoding a protein that elicits an immune response encodes an “antigen,” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

Autologous: As used herein, the term “autologous” refers to any material derived from an individual to which it is later to be re-introduced into the same individual.

Allogeneic: As used herein, the term “allogeneic” refers to any material (e.g., a population of cells) derived from a different animal of the same species.

Xenogeneic: As used herein, the term “xenogeneic” refers to any material (e.g., a population of cells) derived from an animal of a different species.

Cancer: As used herein, the term “cancer” refers to a disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In certain embodiments, the cancer is medullary thyroid carcinoma.

Conservative sequence modifications: As used herein, the term “conservative sequence modifications” refers to amino acid modifications that do not significantly affect or alter the binding characteristics of an antibody containing the amino acid sequence. Such conservative modifications include amino acid substitutions, additions, and deletions. Modifications can be introduced into an antibody compatible with various embodiments by standard techniques known in the art, such as site-directed mutagenesis and PCR-mediated mutagenesis. Conservative amino acid substitutions are ones in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

Co-stimulatory ligand: As used herein, the term “co-stimulatory ligand” refers to a molecule on an antigen presenting cell (e.g., an APC, dendritic cell, B cell, and the like) that specifically binds a cognate co-stimulatory molecule on an immune cell (e.g., a T lymphocyte), thereby providing a signal which mediates an immune cell response, including, but not limited to, proliferation, activation, differentiation, and the like. A co-stimulatory ligand can include, but is not limited to, CD7, B7-1 (CD80), B7-2 (CD86), CD28, PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on an immune cell (e.g., a T lymphocyte), such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

Cytotoxic: As used herein, the term “cytotoxic” or “cytotoxicity” refers to killing or damaging cells. In some embodiments, cytotoxicity of the cells described herein (i.e., cells expressing the IL3-chimeric antigen receptors described herein) is improved, e.g. increased cytolytic activity of immune cells (e.g., T lymphocytes). In some embodiments, cytotoxicity of the cells described herein (i.e., cells expressing the IL3-chimeric antigen receptors described herein) for a target cell expressing an IL3-ligand (i.e., CD123) is improved, e.g. increased cytolytic activity of immune cells (e.g., T lymphocytes).

Effective amount: As used herein, an “effective amount” as described herein refers to a dose that is adequate to prevent or treat cancer in an individual. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that various diseases or disorders could require prolonged treatment involving multiple administrations, for example using the CARS described herein in each or various rounds of administration. By way of example and not intending to limit the invention, when the CARs described herein are provided in a cell expressing the CAR, an exemplary dose of cells may be a minimum of one million cells (1×10⁶ cells/dose).

For purposes of the invention, the amount or dose of an agent comprising an immune cell containing a CAR construct described herein administered should be sufficient to effect a therapeutic or prophylactic response in the subject or animal over a reasonable time frame. For example, the dose should be sufficient to bind to antigen, or detect, treat or prevent a hematopoietic malignancy or pre-malignancy in a period of from about 2 hours or longer, e.g., about 12 to about 24 or more hours, from the time of administration. In certain embodiments, the time period could be even longer. The dose will be determined by the efficacy of the particular CAR described herein and the condition of the animal (e.g., human), as well as the body weight of the animal (e.g., human) to be treated.

Effector function: As used herein, “effector function” or “effector activity” refers to a specific activity carried out by an immune cell in response to stimulation of the immune cell. For example, an effector function of a T lymphocyte includes, recognizing an antigen and killing a cell that expresses the antigen.

Encoding: As used herein, “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Endogenous: As used herein “endogenous” refers to any material from or produced inside a particular organism, cell, tissue, or system.

Exogenous: As used herein, the term “exogenous” refers to any material introduced from or produced outside a particular organism, cell, tissue, or system.

Expand: As used herein, the term “expand” refers to increasing in number, as in an increase in the number of cells, for example, immune cells, e.g., T lymphocytes, NK cells, and/or hematopoietic cells. In some embodiments, immune cells, e.g., T lymphocytes, NK cells, and/or hematopoietic cells that are expanded ex vivo increase in number relative to the number originally present in a culture. In some embodiments, immune cells, e.g., T lymphocytes, NK cells, and/or hematopoietic cells that are expanded ex vivo increase in number relative to other cell types in a culture. In some embodiments, expansion may occur in vivo. The term “ex vivo,” as used herein, refers to cells that have been removed from a living organism, (e.g., a human) and propagated outside the organism (e.g., in a culture dish, test tube, or bioreactor).

Expression: As used herein, the term “expression” of a nucleic acid sequence refers to generation of any gene product from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Expression vector: As used herein, the term “expression vector” or “recombinant expression vector” refers to a vector comprising a recombinant polynucleotide comprising expression control sequences operably linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses).

Fragment: As used herein, the terms “fragment” or “portion” refers to a structure that includes a discrete portion of the whole, but lacks one or more moieties found in the whole structure. In some embodiments, a fragment consists of such a discrete portion. In some embodiments, a fragment consists of or comprises a characteristic structural element or moiety found in the whole. In some embodiments, a nucleotide fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, or more monomeric units (e.g., nucleic acids) as found in the whole nucleotide. In some embodiments, a nucleotide fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or more of the monomeric units (e.g., residues) found in the whole nucleotide. The whole material or entity may in some embodiments be referred to as the “parent” of the fragment.

Functional Portion: As used herein, the term “functional portion” when used in reference to a CAR refers to any part or fragment of the CAR constructs of the invention, which part or fragment retains the biological activity of the CAR construct of which it is a part (the parent CAR construct). Functional portions encompass, for example, those parts of a CAR construct that retain the ability to recognize target cells, or detect, treat, or prevent cancer, such as a hematopoietic malignancy or pre-malignancy, to a similar extent, the same extent, or to a higher extent, as the parent CAR construct. In reference to the parent CAR construct, the functional portion can comprise, for instance, about 10%, about 25%, about 30%, about 50%, about 68%, about 80%, about 90%, about 95%, or more, of the parent CAR.

The functional portion can comprise additional amino acids at the amino or carboxy terminus of the portion, or at both termini, which additional amino acids are not found in the amino acid sequence of the parent CAR construct. Desirably, the additional amino acids do not interfere with the biological function of the functional portion. e.g., recognize target cells, detect cancer, treat or prevent a cancer, such as a hematopoietic malignancy or pre-malignancy, etc. More desirably, the additional amino acids enhance the biological activity as compared to the biological activity of the parent CAR construct.

Functional Variant: As used herein, the term “functional variant.” as used herein, refers to a CAR construct, polypeptide, or protein having substantial or significant sequence identity or similarity to a parent CAR construct, which functional variant retains the biological activity of the CAR of which it is a variant. Functional variants encompass, for example, those variants of the CAR constructs described herein (the parent CAR constructs) that retain the ability to recognize target cells to a similar extent, the same extent, or to a higher extent, as a parent CAR construct. In reference to a parent CAR construct, the functional variant can, for instance, be at least about 30%, about 50%, about 75%, about 80%, about 90%, about 91%, about 92%, about 93%, about 94%, about 95%, about 96%, about 97%, about 98%, about 99% or more identical in amino acid sequence to the parent CAR construct.

A functional variant can, for example, comprise the amino acid sequence of the parent CAR with at least one conservative amino acid substitution. Alternatively or additionally, the functional variants can comprise the amino acid sequence of the parent CAR construct with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR construct.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences. As will be evident to one of ordinary skill in the art, the percent homology may be assessed across the full length of the amino acid or nucleic acid sequences, or a portion thereof (e.g., one or more domains or regions).

Identity: As used herein, the term “identity” refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical. As will be evident to one of ordinary skill in the art, the percent identity may be assessed across the full length of the amino acid or nucleic acid sequences, or a portion thereof (e.g., one or more domains or regions.

Substantial identity: As used herein, the term “substantial identity” refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Immune cell: As used herein, the term “immune cell,” refers to a cell that is involved in an immune response, e.g., promotion of an immune response. Examples of immune cells include, but are not limited to, T-lymphocytes, natural killer (NK) cells, macrophages, monocytes, dendritic cells, neutrophils, cosinophils, mast cells, platelets, large granular lymphocytes, Langerhans' cells, or B-lymphocytes. A source of immune cells (e.g., T lymphocytes) can be obtained from a subject, such as a healthy donor subject or a subject that has or has been diagnosed with a hematopoietic malignancy or pre-malignancy.

Immune response: As used herein the term “immune response” refers to a cellular and/or systemic response to an antigen that occurs when lymphocytes identify antigenic molecules as foreign and induce the formation of antibodies and/or activate lymphocytes to remove the antigen.

Immunoglobulin: As used herein, the term “immunoglobulin” or “Ig.” refers to a class of proteins that function as antibodies. Antibodies expressed by B cells are sometimes referred to as a BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is an immunoglobulin that has no known antibody function, but may serve as an antigen receptor. IgE is an immunoglobulin that mediates immediate hypersensitivity by causing release of mediators from mast cells and basophils upon exposure to allergen.

Isolated: As used herein, the term “isolated” refers to something altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Modified: As used herein, the term “modified” refers to a changed state or structure of a molecule or cell of the invention. Molecules may be modified in many ways, including chemically, structurally, and functionally. Cells may be modified through the introduction of nucleic acids.

Modulating: As used herein the term “modulating,” refers to mediating a detectable increase or decrease in the level of a response and/or a change in the nature of a response in a subject compared with the level and/or nature of a response in the subject in the absence of a treatment or compound, and/or compared with the level and/or nature of a response in an otherwise identical but untreated subject. The term encompasses perturbing and/or affecting a native signal or response thereby mediating a beneficial therapeutic response in a subject, preferably, a human.

Nucleic acid: As used herein, the term “nucleic acid” refers to a polymer of at least three nucleotides. In some embodiments, a nucleic acid comprises DNA. In some embodiments, a nucleic acid comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid.” In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises one or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Operably linked: As used herein, the term “operably linked” refers to functional linkage between, for example, a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

Polynucleotide: As used herein, the term “polynucleotide” refers to a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and polymerase chain reaction (PCR) methods, and the like, and by synthetic means.

Polypeptide: As used herein, the term “polypeptide” refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Signal transduction pathway: As used herein, the term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

Specifically binds: As used herein, the term “specifically binds,” with respect to a ligand, such as an IL-3 molecule or CD123-binding fragment thereof, and its respective receptor (e.g., a specific target antigen), but does not substantially recognize or bind other molecules in a sample, such as other antigens.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, or a dog). In some embodiments a human subject is an adult, adolescent, or pediatric subject. In some embodiments, a subject is suffering from a disease, disorder or condition, e.g., a disease, disorder, or condition that can be treated as provided herein, e.g., a cancer, such as a hematopoietic malignancy or pre-malignancy. In some embodiments, a subject is susceptible to a disease, disorder, or condition; in some embodiments, a susceptible subject is predisposed to and/or shows an increased risk (as compared to the average risk observed in a reference subject or population) of developing the disease, disorder, or condition. In some embodiments, a subject displays one or more symptoms of a disease, disorder, or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered. In some embodiments, the subject has been diagnosed with the disease, disorder, or condition.

Substantially purified: As used herein, the term “substantially purified,” for example as applied to a cell, refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

Target: As used herein, the term “target” refers to a cell, tissue, organ, or site within the body that is the subject of provided methods, systems, and/or compositions, for example, a cell, tissue, organ or site within a body that is in need of treatment or is preferentially bound by, for example, an antibody (or fragment thereof) or a CAR.

Target site: As used herein, the term “target site” or “target sequence” refers to a genomic nucleic acid sequence that defines a portion of a nucleic acid to which a binding molecule (e.g., an IL-3 molecule or a CD123-binding fragment thereof of any of the CARs described herein) may specifically bind under conditions sufficient for binding to occur.

T cell receptor: As used herein, the term “T cell receptor” or “TCR” refers to a complex of membrane proteins that participate in the activation of T cells in response to the presentation of antigen. A TCR is responsible for recognizing antigens bound to major histocompatibility complex molecules. A TCR comprises a heterodimer of an alpha (a) and beta (β) chain, although in some cells the TCR comprises gamma and delta (γ/δ) chains. TCRs may exist in alpha/beta and gamma/delta forms, which are structurally similar but have distinct anatomical locations and functions. Each chain comprises two extracellular domains, a variable and constant domain. In some embodiments, a TCR may be modified on any cell comprising a TCR, including, for example, a helper T cell, a cytotoxic T cell, a memory T cell, regulatory T cell, natural killer T cell, and gamma delta T cell.

Therapeutic: As used herein, the term “therapeutic” refers to a treatment. A therapeutic effect is obtained by suppression, remission, or eradication of a disease state. An effect is obtained by prevention (prophylaxis).

Transfected: As used herein, the term “transfected” or “transformed” or “transduced” refers to a process by which exogenous nucleic acid is transferred or introduced into the host cell. A “transfected” or “transformed” or “transduced” cell is one which has been transfected, transformed, or transduced with exogenous nucleic acid. The cell includes the primary subject cell and its progeny.

Treat: As used herein, the term “treat,” “treatment,” or “treating” refers to partial or complete alleviation, amelioration, delay of onset of, inhibition, relief, and/or reduction in incidence and/or severity of one or more symptoms or features of a disease, disorder, and/or condition. In some embodiments, treatment may be administered to a subject who does not exhibit signs or features of a disease, disorder, and/or condition (e.g., may be prophylactic). In some embodiments, treatment may be administered to a subject who exhibits only early or mild signs or features of the disease, disorder, and/or condition, for example for the purpose of decreasing the risk of developing pathology associated with the disease, disorder, and/or condition. In some embodiments, treatment may be administered to a subject who exhibits established, severe, and/or late-stage signs of the disease, disorder, or condition. In some embodiments, treating may comprise administering to an immune cell (e.g., a T lymphocyte, NK cell) or contacting an immune cell with a modulator of a pathway activated by in vitro transcribed mRNA. In some embodiments, the methods described herein are for prevention of a disease, disorder, and/or condition or one or more symptoms or features of a disease, disorder, and/or condition.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a dispersed tumor or a liquid tumor. In some embodiments, a tumor may be a solid tumor.

Vector: As used herein, the term “vector” refers to a composition of matter that comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2. 2.7, 3, 4, 5, 5.3, and 6. This applies regardless of the breadth of the range.

### DETAILED DESCRIPTION

The present disclosure relates to chimeric antigen receptors (CARs), which may also be referred herein to as “zetakines,” composed of an extracellular domain comprising a surface natural ligand (or variant thereof) of a target antigen that is linked at least to a transmembrane region, and a signaling domain. Zetakines, when expressed on the surface of cells, such as immune effector cells, e.g., T lymphocytes or natural killer (NK) cells, can specifically target malignant cells (e.g., cancer cells) via recognition of the target antigen (e.g., cancer-associated antigen, tumor-associated antigen). Zetakine chimeric immunoreceptors provide an extension of antibody-based immune receptors for redirecting the antigen specificity of T cells. CARs for targeted delivery of oncologic therapeutics have primarily focused on the use of engineered synthetic antibody receptors, which poses a risk of toxicity from T cell hyperactivation. The use of a zetakine containing a natural ligand (or variant thereof) in place of an antibody-based CAR construct further enables structure-guided site-directed mutagenesis to modulate (e.g., increase, decrease) binding affinity and alter cell signaling without hyperactivation (e.g., of T cells) and potential off-target cytotoxicity.

Provided herein are chimeric antigen receptors (e.g., also referred to herein as CARs, zetakines) comprising an interleukin-3 (IL-3) molecule or a CD123-binding fragment thereof; optionally, a linker region; a transmembrane region; optionally, at least one costimulatory signaling domain; and a signaling domain. Also provided herein are nucleic acid constructs and vectors encoding any of the CARs described herein. Also provided herein are cells (e.g., immune cells such as T lymphocytes or NK cells) expressing the CARs. Additionally, the present disclosure provides, in some embodiments, administration of a CAR, a nucleic acid or vector encoding the CAR, or a population of cells that express the CAR to treat a disease or disorder, such as a hematopoictic malignancy or pre-malignancy.

In some aspects, the present disclosure provides methods for treating a disease, disorder, or condition that is characterized by the expression of CD123 on malignant or pre-malignant cells. In some embodiments, the methods involve administering any of the CARs described herein, which target and bind CD123 through an interleukin-3 (IL-3) molecule or a CD123-biding fragment thereof. CD123, also referred to as the IL-3 receptor a chain, has been identified as a favorable therapeutic target for malignancies such as acute myeloid leukemia (AML), and pre-malignant pathologies, such as myelodysplastic syndrome (MDS). CD123 has also been found to be overexpressed in leukemia stem cells (LSCs), progenitor cells, and blast cells.

Acute myeloid leukemia (AML) is an aggressive malignancy that is normally treated using intensive cytotoxic chemotherapeutic regimens with limited alternative therapeutic options when the disease becomes refractory to cytotoxic chemotherapy. Acute myeloid leukemia (AML) is a cancer of the bone marrow that needs more effective therapies. According to the National Cancer Institute, more than 60,000 people in the U.S. have AML, and less than 30% of patients survive five years following diagnosis. Current AML therapies involving targeting CD123 may be effective, potentially limited in utility due to toxicity to healthy cells of the normal blood and bone marrow.

### Chimeric Antigen Receptors

In general, a CAR is an artificially constructed hybrid protein or polypeptide containing an antigen-binding domain, for example of one or more antibodies (e.g., single chain variable fragment (scFv)) linked to T-cell signaling domains. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains. The phrases “antigen(ic) specificity” and “elicit antigen-specific response,” as used herein, means that the CAR can specifically bind to and immunologically recognize antigen, such that binding of the CAR to the target antigen elicits an immune response.

Of the conventional CARs containing an antigen-binding domain of an antibody, there are three generations of CARs. “First generation” CARs are typically composed of an extracellular antigen-binding domain (e.g., a scFv), which is fused to a transmembrane domain, which is fused to cytoplasmic/intracellular signaling domain. First generation CARs can provide de novo antigen recognition and cause activation of both CD4+ and CD8+ T cells through their CD3ζ chain signaling domain in a single fusion molecule, independent of HLA-mediated antigen presentation. “Second generation” CARs add an intracellular signaling domain from various co-stimulatory signaling molecules (e.g., CD28, 4-1BB, ICOS, 0X40, CD27, CD40/My88 and NKGD2) to the cytoplasmic tail of the CAR to provide additional signals to the T cell. Second generation CARs comprise those that provide both co-stimulation (e.g., CD28 or 4-1BB) and activation (CD35). “Third generation” CARs comprise those that provide multiple co-stimulatory molecules (e.g., CD28 and 4-1BB) and a signaling domain providing activation (e.g., CD32).

The CARs described herein, also referred to as “zetakines,” comprise an extracellular portion of the CAR containing an interleukin-3 (IL-3) molecule, or a CD123-binding fragment thereof, rather than an antigen-binding domain of an antibody. The CARs described herein further comprise at least a transmembrane domain and a signaling domain, and optionally, one or more of a linker region, hinge region, and co-stimulatory signaling domains. In some embodiments, the CAR further comprises a signal peptide/signal sequence.

A CAR can consist of or consist essentially of the specified amino acid sequence or sequences described herein, such that other components. e.g., other amino acids, do not materially change the biological activity of the functional variant.

CARs of the present disclosure (including functional portions and functional variants) can be of any length, i.e., can comprise any number of amino acids, provided that the CAR (or functional portions or functional variants thereof) retain their biological activity. e.g., the ability to specifically bind to the target antigen (i.e., CD123), detect diseased cells in a mammal, or treat or prevent disease in a mammal, etc. For example, the CAR can be about 50 to about 5000 amino acids long, such as 50, 70, 75, 100, 125, 150, 175, 200, 300, 400, 500, 600, 700, 800, 900, 1000 or more amino acids in length.

In some embodiments, CAR constructs (including functional portions and functional variants of the invention) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine. 4-nitrophenylalanine, 4-chlorophenylalanine, 4-carboxyphenylalanine, b-phenylserine b-hydroxyphenylalanine, phenylglycine, a-naphthylalanine, cyclohexylalanine, cyclohexylglycine, indoline-2-carboxylic acid, 1,2, 3, 4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, a-aminocycloheptane carboxylic acid, a-(2-amino-2-norbornane)-carboxylic acid, a,g-diaminobutyric acid, a,b-diaminopropionic acid, homophenylalanine, and a-tert-butylglycine.

In some embodiments, CAR constructs (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

In some embodiments, CARs (including functional portions and functional variants thereof) can be obtained by methods known in the art. In some embodiments, the CAR may be made by any suitable method of making polypeptides or proteins, including de novo synthesis. CAR can be recombinantly produced using the nucleic acids described herein using standard recombinant methods. See, for instance, Green et al., Molecular Cloning: A Laboratory Manual, 4th ed., Cold Spring Harbor Press, Cold Spring Harbor, NY 2012. Further, portions of some of the CARs described herein (including functional portions and functional variants thereof) can be isolated and/or purified from a source, such as a plant, a bacterium, an insect, a mammal, e.g., a rat, a human, etc. Methods of isolation and purification are well known in the art. Alternatively, the CARs described herein (including functional portions and functional variants thereof) can be commercially synthesized by companies, such as Synpep (Dublin, CA), Peptide Technologies Corp. (Gaithersburg, MD), and Multiple Peptide Systems (San Diego, CA). In this respect, the CARs can be synthetic, recombinant, isolated, and/or purified.

Further provided herein are nucleic acids comprising a nucleotide sequence encoding any of the CARs described herein (including functional portions and functional variants thereof). The nucleic acids of the invention may comprise a nucleotide sequence encoding any of the leader sequences (e.g., signal peptides), IL-3 molecules or CD123-binding fragments thereof, transmembrane regions, linker regions, costimulatory signaling domains, and/or intracellular T cell signaling domains described herein.

### Interleukin-3

Human interleukin-3 (IL-3) (SEQ ID NO: 1), also referred to as colony-stimulating factor, mast cell growth factor, or multi-CSF, is a 152-residue cytokine primarily produced by activated T lymphocytes and mast cells. It possesses many biological functions, including, for example, as a multicolony-stimulating factor, a histamine-producing cell-stimulating factor, a mast cell growth factor, or a persistent cell-stimulating factor. These broad hematopoietic growth factor activities implicate IL-3 as contributing to leukocyte homeostasis. Binding of IL-3 to CD123 (IL-3 receptor (IL-3R)) results in heterodimer receptor formation, resulting in transmission of IL-3 signaling.

The CARs described herein target CD123 and comprise an extracellular region comprising an IL-3 molecule or a CD123-binding fragment thereof. As used herein, the term “CD123-binding fragment” refers to a portion of a ligand that binds CD123 (e.g., the alpha subunit and/or beta subunit of CD123). A CD123 binding fragment of a protein, such as IL-3, any portion of the protein that binds to CD123 (such as the alpha subunit and/or beta subunit of CD123). In some embodiments, the CD123-binding fragment of an IL3 molecule includes any naturally occurring, enzymatically obtainable, synthetic, or genetically engineered polypeptide fragment of IL-3 that specifically binds CD123 to form a complex.

In some embodiments, the CD123-binding fragment of IL-3 includes at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% 99% or more of an IL-3 (e.g., a naturally occurring IL-3 molecule), such that the fragment retains the ability to bind CD123.

In some embodiments, the IL-3 molecule is human (e.g., obtained from/derived from a human protein sequence). In some embodiments, the IL-3 molecule is a full-length IL-3 molecule, such as a full length human IL-3 molecule. In some embodiments, the IL-3 molecule is a full-length IL-3 molecule provided by SEQ ID NO: 1. In some embodiments, the IL-3 molecule comprises, consists of, or consists essentially of SEQ ID NO: 1.

Human interleukin-3 (NP_000579.2)
(SEQ ID NO: 1)
MSRLPVLLLLQLLVRPGLQAPMTQTTPLKTSWVNCSNMIDEIITHLKQP
PLPLLDFNNLNGEDQDILMENNLRRPNLEAFNRAVKSLQNASAIESILK
NLLPCLPLATAAPTRHPIHIKDGDWNEFRRKLTFYLKTLENAQAQQTTL
SLAIF

In some embodiments, the IL-3 molecule is a portion of an IL-3 molecule, such as the CD123-binding fragment of IL-3 provided by SEQ ID NO: 2. In some embodiments, the IL-3 molecule comprises, consists of, or consists essentially of SEQ ID NO: 2.

Fragment of interleukin-3
(SEQ ID NO: 2)
VNCSNMIDEIITHLKQPPLPLLDENNLNGEDQDILMENNLRRPNLEAFN
RAVKSLQNASAIESILKNLLPCLPLATAAPTRHPIHIKD*GDWNEFRR
K*LTFYLK**TLE

Also within the scope of the present disclosure are variants (e.g., mutants, truncations) of an IL-3 molecule or CD123-binding fragment thereof. For example, one or more mutations (e.g., substitutions) can be made in an IL-3 molecule or CD123-binding fragment thereof to modulate (e.g., increase, decrease) binding of the IL-molecule or CD123-binding fragment thereof to CD123. In some embodiments, the IL-3 molecule, or CD123-binding fragment thereof, comprises a substitution mutation at an amino acid corresponding to position K110 (K* in the above sequence), D101 (D* in the above sequence), K116 (K ** in the above sequence), or a combination thereof. In some embodiments, the IL-3 molecule, or CD123-binding fragment thereof, comprises a substitution of the amino acid corresponding to position K110 to another amino acid residue (e.g., not lysine). In some embodiments, the IL-3 molecule, or CD123-binding fragment thereof, comprises a K110E mutation. In some embodiments, the IL-3 molecule, or CD123-binding fragment thereof, comprises a substitution of the amino acid corresponding to position D101 to another amino acid residue (e.g., not aspartic acid). In some embodiments, the IL-3 molecule, or CD123-binding fragment thereof, comprises a D101A mutation. In some embodiments, the IL-3 molecule, or CD123-binding fragment thereof, comprises a substitution of the amino acid corresponding to position K116 to another amino acid residue (e.g., not lysine). In some embodiments, the IL-3 molecule, or CD123-binding fragment thereof, comprises a K116V mutation or K116W mutation. In some embodiments, the IL-3 molecule, or CD123-binding fragment thereof, comprises a D101A mutation and a K116V mutation.

In some embodiments, the IL-3 molecule is a portion of an IL-3 molecule containing a K110E mutation provided by SEQ ID NO: 3. In some embodiments, the IL-3 molecule comprises, consists of, or consists essentially of SEQ ID NO: 3. The mutation relative to SEQ ID NO: 2 is indicated in boldface with underling.

Fragment of interleukin-3 (K110E mutant)
(SEQ ID NO: 3)
VNCSNMIDEIITHLKQPPLPLLDFNNLNGEDQDILMENNLRRPNLEAFN
RAVKSLQNASAIESILKNLLPCLPLATAAPTRHPIHIKDGDWNEFRREL
TFYLKTLE

In some embodiments, the IL-3 molecule is a portion of an IL-3 molecule containing a D101A mutation and K116V provided by SEQ ID NO: 4. In some embodiments, the IL-3 molecule comprises, consists of, or consists essentially of SEQ ID NO: 4. The mutations relative to SEQ ID NO: 2 are indicated in boldface with underling.

Fragment of interleukin-3 (D101A, K116V mutant)
(SEQ ID NO: 4)
VNCSNMIDEIITHLKQPPLPLLDFNNLNGEDQDILMENNLRRPNLEAFN
RAVKSLQNASAIESILKNLLPCLPLATAAPTRHPIHIKAGDWNEFRRKL
TFYLVTLE

In some embodiments, the IL-3 molecule is a portion of an IL-3 molecule containing a K116W provided by SEQ ID NO: 5. In some embodiments, the IL-3 molecule comprises, consists of, or consists essentially of SEQ ID NO: 5. The mutation relative to SEQ ID NO: 2 is indicated in boldface with underling.

Fragment of interleukin-3 (K116W mutant)
(SEQ ID NO: 5)
VNCSNMIDEIITHLKQPPLPLLDFNNLNGEDQDILMENNLRRPNLEAFN
RAVKSLQNASAIESILKNLLPCLPLATAAPTRHPIHIKDGDWNEFRRKL
TFYLWTLE

In some aspects, the IL-3 molecule or CD123-binding fragment thereof is operably linked to another domain of the CAR, such as a linker region, a hinge domain, a transmembrane region, or an intracellular domain (e.g., a costimulatory signaling domain, a signaling domain) for expression in a cell. In some embodiments, a nucleic acid encoding the IL-3 molecule, or CD123-binding fragment thereof, is operably linked to a nucleic acid encoding a transmembrane domain and a nucleic acid encoding an intracellular domain (e.g., a costimulatory signaling domain, a signaling domain). In some embodiments, a nucleic acid encoding the IL-3 molecule, or CD123-binding fragment thereof, is operably linked to a nucleic acid encoding a linker region, a nucleic acid encoding a transmembrane domain, and a nucleic acid encoding an intracellular domain (e.g., a costimulatory signaling domain, a signaling domain).

In some embodiments, the CAR comprises a linker region. In some embodiments, the linker region is a Gly/Ser linker from about 1 to about 100, from about 3 to about 20, from about 5 to about 30, from about 5 to about 18, or from about 3 to about 8 amino acids in length and consists of glycine and/or serine residues in sequence. Accordingly, the Gly/Ser linker may consist of glycine and/or serine residues. Preferably, the Gly/Ser linker comprises the amino acid sequence of GGGGS (SEQ ID NO: 6), and multiple repeats of the sequence provided by SEQ ID NO: 6 may be present within the linker. Any linker sequence may be used as a spacer between IL-3 molecule or CD123-binding fragment thereof and any other domain of the CAR, such as the transmembrane domain.

In some, embodiments, the region linker is ([G]x[S]y) z, for example wherein x can be 1-10, y can be 1-3, and z can be 1-5. In some embodiments, the linker region comprises the amino acid sequence GGGGSGGGGS (SEQ ID NO: 7).

In an embodiment, IL-3 molecule, or CD123-binding fragment thereof, comprises one or more leader sequences (signal peptides, signal sequence), such as those described herein. In some embodiments, the signal peptide may be positioned at the amino terminus (N-terminus) of the CAR within the CAR construct. The leader sequence may comprise any suitable leader sequence, e.g., any CAR described herein may comprise any signal peptide as described herein. In some embodiments, while the signal peptide may facilitate expression of the released CARs on the surface of the cell, the presence of the signal peptide in an expressed CAR is not necessary in order for the CAR to function. In some embodiments, upon expression of the CAR on the cell surface, the signal peptide may be cleaved off. Accordingly, in some embodiments, the released CARs lack a signal peptide. In some embodiments, the CARs within the CAR construct lack a signal peptide.

### Hinge

In some embodiments, the CAR also comprises a hinge/spacer region that links the extracellular antigen-binding domain (e.g., IL-3 molecule or a CD123-binding fragment thereof) to another domain, such as a transmembrane domain. The hinge/spacer region can be flexible enough to allow the antigen-binding domain to orient in different directions to facilitate target antigen recognition.

In some embodiments, the CAR comprises a hinge domain, such as a hinge domain from CD8, CD28, or IgG4. In some embodiments, the hinge domain is a CD8 (e.g., CD8a) hinge domain. In some embodiments, the CD8 hinge domain is human (e.g., obtained from/derived from a human protein sequence). In some embodiments, the CD8 hinge domain comprises, consists of, or consists essentially of SEQ ID NO: 8.

CD8 hinge region
(SEQ ID NO: 8)
(SEQ ID NO: 8)
TTTPAPRPPTPAPTIASQPLSLRPEACRPAAGGAVHTRGLDFACD

In some embodiments, the hinge domain is a CD28 hinge domain. In some embodiments, the CD28 hinge domain is human. In some embodiments, the CD28 hinge domain is human (e.g., obtained from/derived from a human protein sequence). In some embodiments, the CD28 hinge domain comprises, consists of, or consists essentially of SEQ ID NO: 9.

CD28 hinge region
(SEQ ID NO: 9)
AAAIEVMYPPPYLDNEKSNGTIIHVKGKHLCPSPLFPGPSKP

Hinge domains of antibodies, such as an IgG, IgA, IgM, IgE, or IgD antibody, are also compatible for use in the chimeric receptors described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody. In some embodiments, the hinge domain is an IgG4 hinge domain.

In some embodiments, the hinge domain is a IgG4 hinge domain. In some embodiments, the IgG4 hinge domain is human. In some embodiments, the IgG4 hinge domain is human (e.g., obtained from/derived from a human protein sequence). In some embodiments, the IgG4 hinge domain is an Fc fragment (or portion thereof) of an IgG4 antibody. In some embodiments, the IgG4 hinge domain is a human immunoglobulin subclass G4 Fc fragment (or portion thereof) of an IgG4 antibody. In some embodiments, the IgG4 hinge domain comprises, consists of, or consists essentially of SEQ ID NO: 10.

IgG4 hinge region
(SEQ ID NO: 10)
ESKYGPPCPPCPAPEFL*GGPSVFLFPPKPKDTLMISRTPEVTCVVVDV
SQEDPEVQFNWYVDGVEVHN*AKTKPREEQFNSTYRVVSVLTVLHQDWL
NGKEYKCKVSNKGLPSSIEKTISKAKGQPREPQVYTLPPSQEEMTKNQV
SLTCLVKGFYPSDIAVEWESNGQPENNYKTTPPVLDSDGSFFLYSRLTV
DKSRWQEGNVFSCSVMHEALHNHYTQKSLSLSLGK

In some embodiments, the hinge domain is a portion of the hinge domain of CD8a, CD28, or IgG4, e.g., a fragment containing at least 15 (e.g., 20, 25, 30, 35, or 40) consecutive amino acids of the hinge domain of CD8a, CD28, or IgG4.

In some embodiments, the hinge/spacer region of any of the CARs described herein comprises a native or modified hinge region of a CD28 polypeptide. In some embodiments, the hinge/spacer region of any of the CARs described herein comprises a native or modified hinge region of a CD8a polypeptide. In some embodiments, the hinge/spacer region of any of the CARs described herein comprises a native or modified hinge region of a IgG4 polypeptide as described herein.

Also within the scope of the present disclosure are variants (e.g., mutants, truncations) of a hinge/space regions. For example, one or more mutations (e.g., substitutions) can be made in hinge/space regions to modulate (e.g., increase, decrease) binding of the IL-molecule or CD123-binding fragment thereof to CD123. In some embodiments, the hinge/space region is a IgG4 hinge and comprises a substitution mutation at an amino acid corresponding to position L235 (L* in the sequence above), N297 (N* in the sequence above), or a combination thereof. In some embodiments, the hinge/space region comprises a substitution of the amino acid corresponding to position L325 to another amino acid residue (e.g., not leucine). In some embodiments, the hinge/space region comprises a L235E mutation. In some embodiments, the hinge/space region comprises a substitution of the amino acid corresponding to position N297 to another amino acid residue (e.g., not asparagine). In some embodiments, the hinge/space region comprises a N297Q mutation.

In some embodiments, the hinge/space region comprises a L235E mutation and a N297Q mutation, as provided by SEQ ID NO: 11. In some embodiments, the hinge/spacer region comprises, consists of, or consists essentially of SEQ ID NO: 11. The mutations relative to SEQ ID NO: 10 are indicated in boldface with underlining.

IgG4 hinge region (L235E, N297Q mutant)
(SEQ ID NO: 11)
ESKYGPPCPSCPAPEFEGGPSVFLFPPKPKDTLMISRTPEVTCVVVDVS
QEDPEVQFNWYVDGVEVHQAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK

Also within the scope of the present disclosure are CARs comprising a hinge domain that is a non-naturally occurring peptide. In some embodiments, the hinge domain between the C-terminus of the extracellular ligand-binding domain of an Fc receptor and the N-terminus of the transmembrane domain is a peptide linker, such as a (GlyₓSer)ₙ linker, wherein x and n, independently can be an integer between 3 and 12, including 3, 4, 5, 6, 7, 8, 9, 10, 11, 12, or more.

Additional peptide linkers that may be used in a hinge domain of the chimeric receptors described herein are known in the art. See, e.g., Wriggers et al. Current Trends in Peptide Science (2005) 80 (6): 736-74 and PCT Publication No. WO 2012/088461, which are incorporated by reference herein.

### Transmembrane Region

With respect to the transmembrane region, a CAR can be designed to comprise a transmembrane region that connects the IL-3 molecule, or CD123-binding fragment thereof, of the CAR to the intracellular region of the CAR. In some embodiments, the transmembrane domain is naturally associated with one or more of the domains in the CAR. In some instances, the transmembrane domain can be selected or modified by amino acid substitution to avoid binding of such domains to the transmembrane regions of the same or different surface membrane proteins to minimize interactions with other members of the receptor complex.

The transmembrane domain may be derived either from a natural source or from a synthetic source. Where the source is natural, the domain may be derived from any membrane-bound or transmembrane protein. Transmembrane regions for particular use may be derived from (i.e., comprise at least the transmembrane region(s) of the alpha, beta or zeta chain of the T-cell receptor, CD28, CD3 epsilon, CD45, CD4, CD5, CD8, CD8a, CD9, CD 16, CD22, CD33, CD37, CD64, CD80, CD86, CD134, CD137, CD154, Toll-like receptor 1 (TLR1), TLR2, TLR3, TLR4, TLR5, TLR6, TLR7, TLR8, and TLR9).

In one embodiment, the transmembrane domain may be synthetic, in which case it will comprise predominantly hydrophobic residues such as leucine and valine. Preferably a triplet of phenylalanine, tryptophan and valine will be found at each end of a synthetic transmembrane region.

In some embodiments, the transmembrane region is a CD8 (e.g., CD8a) transmembrane region. In some embodiments, the CD8 transmembrane region is human (e.g., obtained from/derived from a human protein sequence). In some embodiments, a CD8 transmembrane region comprises, consists of, or consists essentially of SEQ ID NO: 12.

CD8 transmembrane region
(SEQ ID NO: 12)
IYIWAPLAGTCGVLLLSLVITLYC

In some embodiments, the transmembrane region is a CD28 transmembrane region. In some embodiments, the CD28 transmembrane region is human (e.g., obtained from/derived from a human protein sequence). In some embodiments, the CD28 transmembrane region comprises, consists of, or consists essentially of SEQ ID NO: 13.

CD28 transmembrane region
(SEQ ID NO: 13)
FWVLVVVGGVLACYSLLVTVAFIIFWVRSKRSRLLHSDYMNMTPRRPGP
TRKHYQPYAPPRDFAAYRS

In some embodiments, the transmembrane region is a CD4 transmembrane region. In some embodiments, the CD4 transmembrane region is human (e.g., obtained from/derived from a human protein sequence). In some embodiments, the CD4 transmembrane region comprises, consists of, or consists essentially of SEQ ID NO: 14.

CD4 transmembrane region
(SEQ ID NO: 14)
MALIVLGGVAGLLLFIGLFIFF

### Intracellular Signaling Domains

In some embodiments, a CAR construct comprises an intracellular signaling domain, which may be comprised of one or more signaling domains and costimulatory signaling domains. The intracellular signaling domain of the CAR, is involved in activation of the cell in which the CAR is expressed. The intracellular signaling domain of the CAR, is involved in activation of a T lymphocyte or NK cell. In some embodiments, the signaling domain of the CAR construct described herein includes a domain involved in for signal activation and/or transduction.

Examples of an intracellular signaling domains for use in the CAR constructs described herein include, but are not limited to, the cytoplasmic portion of a surface receptor, co-stimulatory molecule, and any molecule that acts in concert to initiate signal transduction in a cell (e.g., an immune cell (e.g., a T lymphocyte, NK cell)), as well as any derivative or variant of these elements and any synthetic sequence that has the same functional capability.

Examples of the signaling domains that may be used in the intracellular signaling domain of the CARs described herein include, without limitation, a fragment or domain from one or more molecules or receptors including, but are not limited to, TCR, CD3 zeta (CD35), CD3 gamma, CD3 delta, CD3 epsilon, CD86, common FcR gamma, FcR beta (Fc Epsilon Rib), CD79a, CD79b, Fcgamma RIIa, DAP10, DAP 12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD 1 id, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAMI (CD226), SLAMF4 (CD244, 2B4), CD84, CD96 (Tactile), CEACAMI, CRTAM, Ly9 (CD229), CD160 (BY55), PSGLI, CD100 (SEMA4D), CD69, SLAMF6 (NTB-A, Ly108), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Any cytoplasmic signaling domain can be used in the CARs described herein. In general, a cytoplasmic signaling domain relays a signal, such as interaction of an extracellular ligand-binding domain with its ligand, to stimulate a cellular response, such as inducing an effector function of the cell (e.g., cytotoxicity).

As will be evident to one of ordinary skill in the art, a factor involved in T cell activation is the phosphorylation of immunoreceptor tyrosine-based activation motif (ITAM) of a cytoplasmic signaling domain. Any ITAM-containing domain known in the art may be used to construct the chimeric receptors described herein, and included as part of the cytoplasmic signaling domain. In general, an ITAM motif may comprise two repeats of the amino acid sequence YxxL/I (SEQ ID NO: 56) separated by 6-8 amino acids, wherein each x is independently any amino acid, producing the conserved motif YxxL/Ix (6-8) YxxL/I (SEQ ID NO: 57). In some embodiments, the cytoplasmic signaling domain is from CD35 (CD3 zeta).

CD3ζ associates with TCRs to produce a signal and contains immunoreceptor tyrosine-based activation motifs (ITAMs). In some embodiments, a CD3ζ intracellular T cell signaling sequence is human. In some embodiments, a CD3ζ intracellular T cell signaling sequence comprises, consists of, or consists essentially of the amino acid sequence of SEQ ID NO: 15 or 16, or a sequence that is at least 70%, at least 75%, at least 80%, at least 85%, at least 90%, at least 95%, or at least 99% identical the amino acid sequence of SEQ ID NO: 15 or 16. In some embodiments, an intracellular T cell signaling domain comprises a CD35 intracellular T cell signaling domain that contains on or more mutated and/or deleted ITAMs. One of ordinary skill in the art would recognize that variants (e.g., mutants) CD3° C. intracellular T cell signaling sequence are also suitable for the CARs described herein, such as the exemplary variants shown below.

CD3ζ signaling domain (variant A)
(SEQ ID NO: 15)
RVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR
CD3ζ signaling domain (variant B)
(SEQ ID NO: 16)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In certain non-limiting embodiments, an intracellular signaling domain of the CAR further comprises at least one (e.g., 1, 2, 3 or more) co-stimulatory signaling domain. In some embodiments, the co-stimulatory signaling domain comprises at least one co-stimulatory molecule, which can provide optimal lymphocyte activation. In general, many immune cells require co-stimulation, in addition to stimulation of an antigen-specific signal, to promote cell proliferation, differentiation and survival, and to activate effector functions of the cell. Activation of a co-stimulatory signaling domain in a cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory protein may be compatible for use in the chimeric receptors described herein. The type(s) of co-stimulatory signaling domains may be selected based on factors such as the type of cells in which the CARs are to be expressed (e.g., primary T cells, T cell lines, NK cell lines) and the desired immune effector function (e.g., cytotoxicity).

Examples of such co-stimulatory signaling domains include a fragment or domain from one or more molecules or receptors including, without limitation, 4-1BB, CD28, ICOS. TLR1, TLR2, TLR3, TLR4, TLR5, TLR6, TLR7, TLR8, TLR9, TLR10, TLR11, CD116 receptor beta chain, CSF1-R, LRP1/CD91, SR-A1, SR-A2, MARCO, SR-CL1, SR-CL2, SR—C, SR-E, CR1, CR3, CR4, dectin 1. DEC-205, DC-SIGN, CD14, CD36, LOX-1, CD11b, together with any of the signaling domains listed in the above paragraph in any combination. In some embodiments, the intracellular signaling domain of the CAR includes any portion of one or more co-stimulatory signaling molecules, such as at least one signaling domain from CD3, Fc epsilon RI gamma chain, or any derivative or variant thereof, including any synthetic sequence thereof that has the same functional capability, and any combination thereof.

In some embodiments, one or more co-stimulatory signaling domains (e.g., 1, 2, 3, or more) are included in a CAR construct with a CD3 intracellular T cell signaling sequence. In some embodiments, the one or more co-stimulatory signaling domains are selected from 4-1BB (CD137), OX-40, and CD28, or a combination thereof. In some embodiments, the CAR comprises a 4-1BB (CD137) costimulatory signaling domain. In some embodiments, the CAR comprises an OX-40 costimulatory signaling domain. In some embodiments, the CAR comprises a CD28 costimulatory signaling domain.

4-1BB, also known as CD137, transmits a potent costimulatory signal to T cells, promoting differentiation and enhancing long-term survival of T lymphocytes. In some embodiments, a 4-1BB intracellular signaling sequence is human (e.g., obtained from/derived from a human protein sequence). In some embodiments, the 4-1BB intracellular T cell signaling sequence comprises, consists of, or consists essentially of the amino acid sequence of SEQ ID NO: 17. In some embodiments, the 4-1BB costimulatory signaling domain comprises, consists of, or consists essentially of the amino acid sequence of SEQ ID NO:17, or a sequence that is at least 70%, at least 75%, at least 80%, at least 85%, at least 90%, at least 95%, or at least 99% identical the amino acid sequence of SEQ ID NO: 17.

4-1BB costimulatory signaling domain
(SEQ ID NO: 17)
KRGRKKLLYIFKQPFMRPVQTTQEEDGCSCRFPEEEEGGCEL

In some embodiments, the costimulatory signaling domain is a CD28 costimulatory signaling domain. In some embodiments, the CD28 costimulatory signaling domain comprises, consists of, or consists essentially of the amino acid sequence of SEQ ID NO: 18. In some embodiments, the CD28 costimulatory signaling domain comprises, consists of, or consists essentially of the amino acid sequence of SEQ ID NO: 18, or a sequence that is at least 70%, at least 75%, at least 80%, at least 85%, at least 90%, at least 95%, or at least 99% identical the amino acid sequence of SEQ ID NO: 18.

CD28 costimulatory signaling domain
(SEQ ID NO: 18)
RSKRSRLLHSDYMNMTPRRPGPTRKHQYPYAPPRDFAAYRS

Between the antigen-binding domain (e.g., IL-3 molecule or CD123-binding fragment thereof) and the transmembrane domain of the CAR, or between the intracellular signaling domain and the transmembrane domain of the CAR, a spacer domain may be incorporated. As used herein, the term “spacer domain” generally means any oligo- or polypeptide that functions to link the transmembrane domain to, either the antigen binding domain or, the intracellular domain in the polypeptide chain. In one embodiment, the spacer domain may comprise up to 300 amino acids, preferably 10 to 100 amino acids and most preferably 25 to 50 amino acids. In another embodiment, a short oligo- or polypeptide linker, preferably between 2 and 10 amino acids in length may form the linkage between the transmembrane domain and the intracellular domain of the CAR. An example of a linker includes a glycine-serine doublet.

### Signal Peptides

In some embodiments, any of the CARs described herein may further comprise a signal peptide (signal sequence). In general, signal peptides are short amino acid sequences that target a polypeptide to a site in a cell. In some embodiments, the signal peptide directs the CAR to the secretory pathway of the cell and will allow for integration and anchoring of the CAR into the lipid bilayer at the cell surface. Signal sequences including signal sequences of naturally occurring proteins or synthetic, non-naturally occurring signal sequences, that are compatible for use in the chimeric receptors described herein will be evident to one of skill in the art.

In some embodiments, the signal peptide is a GM-CSF signal peptide. In some embodiments, the GM-CSF signal peptide is human (e.g., obtained from/derived from a human protein sequence). In some embodiments, the GM-CSF signal peptide comprises, consists of, or consists essentially of the amino acid sequence of SEQ ID NO: 19. In some embodiments, the GM-CSF signal peptide comprises, consists of, or consists essentially of the amino acid sequence of SEQ ID NO: 19, or a sequence that is at least 70%, at least 75%, at least 80%, at least 85%, at least 90%, at least 95%, or at least 99% identical the amino acid sequence of SEQ ID NO: 19.

GM-CSF signal peptide
(SEQ ID NO: 19)
MLLLVTSLLLCELPHPAFLLI

The CARs described herein may be prepared in constructs with, e.g., self-cleaving peptides, such that the CAR constructs containing the components are bicistronic, tricistronic, etc.

### Vectors

Nucleic acids encoding the CAR constructs described herein can be incorporated into a vector, such as a recombinant expression vector. As used herein, the terms “recombinant expression vector” and “vector” may be used interchangeably and refer to a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell.

In some embodiments, vectors are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. In some embodiments, the vector is a DNA vector. In some embodiments, the vector is a RNA vector. The vectors can comprise naturally-occurring or non-naturally-occurring internucleotide linkages, or both types of linkages. In some embodiments, a non-naturally occurring or altered nucleotides or internucleotide linkages do not hinder the transcription or replication of the vector.

The vector may be any suitable recombinant expression vector, and can be used to transform or transfect any suitable host cell. Suitable vectors include those designed for propagation and expansion or for expression or both, such as plasmids and viruses. A vector can be selected from the group consisting of the pUC series (Fermentas Life Sciences, Glen Burnie, MD), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as LGTIO, λGT11, LZapII (Stratagene), λEMBT4, and λNMI149, also can be used. Examples of plant expression vectors include pBIO1, pBI101.2, pBI101.3, pBH21 and pBIN19 (Clontech). Examples of animal expression vectors include pEUK-CI, pMAM, and pMAMneo (Clontech). The recombinant expression vector may be a viral vector, e.g., an adenoviral vector, a retroviral vector, or a lentiviral vector. In some embodiments, the vector is an adenoviral vector. In some embodiments, the vector is a retroviral vector. In some embodiments, the vector is a lentiviral vector.

In some embodiments, the vectors can be prepared using standard recombinant DNA techniques described in, for example, Green et al., supra. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

A recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. A vector may also comprise restriction sites to facilitate cloning.

A vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

Further, the vectors can be made to include a suicide gene. As used herein, the term “suicide gene” refers to a gene that causes the cell expressing the suicide gene to die. A suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

### Promoters

In some embodiments, a recombinant expression vector can comprise a native or nonnative promoter operably linked to the nucleotide sequence encoding the CAR construct (including functional portions and functional variants thereof), or to the nucleotide sequence which is complementary to or which hybridizes to the nucleotide sequence encoding the CAR construct. The selection of promoters, e.g., strong, weak, inducible, constitutive, tissue-specific, and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, a SFFV promoter, an EF1α promoter, an SV40 promoter, an RSV promoter, or a promoter found in the long-terminal repeat of the murine stem cell virus. In some embodiments the promoter is an SFFV promoter (e.g., as represented in SEQ ID NO: 20).

SFFV promoter
(SEQ ID NO: 20)
GTAACGCCATTTTGCAAGGCATGGAAAAATACCAAACCAAGAATAGAGA
AGTTCAGATCAAGGGCGGGTACATGAAAATAGCTAACGTTGGGCCAAAC
AGGATATCTGCGGTGAGCAGTTTCGGCCCCGGCCCGGGGCCAAGAACAG
ATGGTCACCGCAGTTTCGGCCCCGGCCCGAGGCCAAGAACAGATGGTCC
CCAGATATGGCCCAACCCTCAGCAGTTTCTTAAGACCCATCAGATGTTT
CCAGGCTCCCCCAAGGACCTGAAATGACCCTGCGCCTTATTTGAATTAA
CCAATCAGCCTGCTTCTCGCTTCTGTTCGCGCGCTTCTGCTTCCCGAGC
TCTATAAAAGAGCTCACAACCCCTCACTCGGCGCGCCAGTCCTCCGACA
GACTGAGTCG

The vectors described herein can be designed for transient expression, stable expression, or for both. Alternatively or in addition, the vectors can be made for constitutive expression or for inducible expression.

Included in the scope of the invention are conjugates, e.g., bioconjugates, comprising any of the CAR constructs (including any of the functional portions or variants thereof), nucleic acids, recombinant expression vectors, host cells, or populations of host cells described herein. Conjugates, as well as methods of synthesizing conjugates in general, are known in the art.

### Production of Modified Cells

Aspects of the present disclosure provide methods for modifying a cell comprising introducing a chimeric antigen receptor (CAR) into a cell (e.g., an immune cell such as a T lymphocyte or NK cell), wherein the CAR comprises an IL-3 molecule, or CD123-binding fragment thereof, a transmembrane domain, and a signaling domain, wherein the CAR may further optionally comprise a linker region, a hinge region, and/or at least one costimulatory domains. In some embodiments, the cell wherein the cell expresses the CAR and possesses targeted effector activity. In some embodiments, introducing the CAR into the cell comprises introducing a nucleic acid sequence encoding the CAR. In another embodiment, introducing the nucleic acid sequence comprises electroporating a mRNA encoding the CAR into the cell.

In some embodiments, the cell may be an immune cell such as a T lymphocyte or NK cell. A T lymphocyte can be any T cell, such as a cultured T cell, e.g., a primary T cell, or a T cell from a cultured T cell line, e.g., TIB-153™, Jurkat, SupTI, etc., or a T cell obtained from a mammal. If obtained from a mammal, a T cell can be obtained from numerous sources, including but not limited to blood, bone marrow, lymph node, the thymus, or other tissues or fluids. T cells can also be enriched for or purified. In some embodiments, the T cell is a human T cell. In some embodiments, the T cell may be a T cell isolated from a human. A T cell can be any type of T cell and can be of any developmental stage, including but not limited to, CD4+/CD8+double positive T cells, CD4+ helper T cells, e.g., Th1 and Th2 cells, CD8+ T cells (e.g., cytotoxic T cells), tumor infiltrating cells, memory T cells, naive T cells, and the like. A T cell may be a CD8+ T cell or a CD4+ T cell. In some embodiments, the T cell is an alpha/beta T cell. In some embodiments, the T cell is a gamma/delta T cell. In some embodiments, the immune cell is a natural killer T cell (NKT cell). In some embodiments, the immune cell is a natural killer cell (NK cell).

Methods of introducing and expressing genes, such as the CAR, into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, transduction (e.g., lentiviral transduction, retroviral transduction), electroporation (e.g., DNA or RNA electroporation), and the like. Methods for producing cells comprising vectors and/or exogenous nucleic acids are well known in the art. See, for example, Sambrook et al., 2012, MOLECULAR CLONING: A LABORATORY MANUAL, Volumes 1-4, Cold Spring Harbor Press, NY). Nucleic acids can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). Nucleic acids can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12 (8): 861-70 (2001).

In one aspect, a DNA or RNA construct is introduced into the cells by electroporation. Sec, e.g., the formulations and methodology of electroporation of nucleic acid constructs into mammalian cells as taught in U.S. Publication Nos. U.S. 2004/0014645, U.S. 2005/0052630A1. U.S. 2005/0070841 A1, U.S. 2004/0059285A1, U.S. 2004/0092907A1, which are incorporated by reference herein. The various parameters including electric field strength required for electroporation of any known cell type are generally known in the relevant research literature as well as numerous patents and applications in the field. See e.g., U.S. Pat. Nos. 6,678,556, 7,171,264, and 7,173,116, which are incorporated by reference herein. Apparatuses for therapeutic application of electroporation are available commercially, e.g., the MedPulser™ DNA Electroporation Therapy System (Inovio/Genetronics, San Diego, Calif), and are described in patents such as U.S. Pat. Nos. 6,567,694; 6,516,223, 5,993,434, U.S. Pat. Nos. 6,181,964, 6,241,701, and 6,233,482; electroporation may also be used for transfection of cells in vitro as described e.g. in U.S. Publication No. U.S. 2007/0128708A1, each of which are incorporated by reference herein. Electroporation may also be utilized to deliver nucleic acids into cells in vitro. Accordingly, electroporation-mediated administration into cells of nucleic acids including expression constructs utilizing any of the many available devices and electroporation systems known to those of skill in the art present additional means for delivering a DNA or RNA of interest to a target cell.

Biological methods for introducing a polynucleotide of interest into a host cell include the use of DNA and RNA vectors. RNA vectors include vectors having an RNA promoter and/other relevant domains for production of a RNA transcript. Viral vectors, and especially retroviral vectors, have become the most widely used method for inserting genes into mammalian, e.g., human cells. Other viral vectors may be derived from lentivirus, poxviruses, herpes simplex virus, adenoviruses and adeno-associated viruses, and the like. See, for example, U.S. Pat. Nos. 5,350,674 and 5,585,362, which are incorporated by reference herein.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo, or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., (1991) Glycobiology 5:505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a cell or otherwise expose a cell to the molecules described herein, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention. In some embodiments, the methods further involve selecting the cells in which the exogenous nucleic acids have been introduced (and expressed) from a population of cells, such as through use of a selectable marker.

### CAR Constructs

In some embodiments, a CAR includes particular components including an IL-3 molecule, or CD123-binding fragment thereof, a transmembrane domain, and a signaling domain. In some embodiments, the CAR further comprises one or more of a linker region, hinge region, and/or one or more costimulatory signaling domains. A CAR may include any combinations of the exemplary elements described herein, for example, any of the IL-3 molecules or CD123-binding fragments thereof, transmembrane domains, hinge domains, signaling domains, and any one or more co-stimulatory signaling domains described here. In some embodiments, any of the CARs described herein may further comprise a signal peptide (signal sequence).

In some embodiments, the CAR comprises, from N-terminus to C-terminus: (a) the interleukin-3 (IL-3) molecule or a CD123-binding fragment thereof; (b) the linker region, (c) the transmembrane region, and (d) the signaling domain. In some embodiments, the CAR does not comprise a costimulatory signaling domain. In some embodiments, the CAR further comprises a signal peptide/signal sequence at the N-terminus of the CAR, which may be removed from the protein upon surface presentation.

In some embodiments, the CAR comprises, from N-terminus to C-terminus: (a) the interleukin-3 (IL-3) molecule or a CD123-binding fragment thereof; (b) the linker region, (c) the transmembrane region, and (d) the one or more co-stimulatory signaling domains, and (e) the signaling domain. In some embodiments, the CAR further comprises a signal peptide/signal sequence at the N-terminus of the CAR, which may be removed from the protein upon surface presentation.

Amino acid sequences of exemplary CARs are provided below along with exemplary nucleic acid sequences for encoding the CARs.

### 1. IL3-WT-Nocostim

An exemplary CAR construct, as described herein, comprises a CD123-binding fragment of IL-3, a IgG4 hinge domain, a CD4 transmembrane domain, and a CD3ζ intracellular signaling domain. In some embodiments, the CAR may further comprise a signal peptide, such as the exemplary signal peptide provided by SEQ ID NO: 19.

In some embodiments, a CAR comprises an amino acid sequence shown in SEQ ID NO: 21, or an amino acid sequence that is at least 70%, at least 75%, at least 80%, at least 85%, at least 90%, at least 95%, or at least 99% identical to the amino acid sequence shown in SEQ ID NO: 21.

Amino acid sequence of IL3-WT-nocostim
(SEQ ID NO: 21)
VNCSNMIDEIITHLKQPPLPLLDFNNLNGEDQDILMENNLRRPNLEAFN
RAVKSLQNASAIESILKNLLPCLPLATAAPTRHPIHIKDGDWNEFRRKL
TFYLKTLEESKYGPPCPPCPAPEFLGGPSVFLFPPKPKDTLMISRTPEV
TCVVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFL
YSRLTVDKSRWQEGNVFSCSVMHEALHNHYTQKSLSLSLGKMALIVLGG
VAGLLLFIGLGIFFRVKFSRSADAPAYQQGQNQLYNELNLGRREEYDVL
DKRRGRDPEMGGKPRRKNPQEGLYNELQKDKMAEAYSEIGMKGERRRGK
GHDGLYQGLSTATKDTYDALHMQALPPR

In some embodiments, a CAR in encoded by a nucleic acid sequence that comprises the sequence that is shown in SEQ ID NO: 22, or in a nucleic acid sequence that is at least 70%, at least 75%, at least 80%, at least 85%, at least 90%, at least 95%, or at least 99% identical to the nucleic acid sequence shown in SEQ ID NO: 22.

Nucleic acid sequence of IL3-WT-nocostim
(SEQ ID NO: 22)
GTGAACTGCAGCAACATGATCGACGAGATCATCACCCACCTGAAGCAGCCCCCCCTGCC
CCTGCTGGACTTCAACAACCTGAACGGCGAGGACCAGGACATCCTGATGGAGAACAACC
TGAGGAGGCCCAACCTGGAGGCCTTCAACAGGGCCGTGAAGAGCCTGCAGAACGCCAGC
GCCATCGAGAGCATCCTGAAGAACCTGCTGCCCTGCCTGCCCCTGGCCACCGCCGCCCCC
ACCAGGCACCCCATCCACATCAAGGACGGCGACTGGAACGAGTTCAGGAGGAAGCTGAC
CTTCTACCTGAAGACCCTGGAGGAGAGCAAGTACGGCCCCCCCTGCCCCCCCTGCCCCGC
CCCCGAGTTCCTGGGCGGCCCCAGCGTGTTCCTGTTCCCCCCCAAGCCCAAGGACACCCT
GATGATCAGCAGGACCCCCGAGGTGACCTGCGTGGTGGTGGACGTGAGCCAGGAGGACC
CCGAGGTGCAGTTCAACTGGTACGTGGACGGCGTGGAGGTGCACAACGCCAAGACCAAG
CCCAGGGAGGAGCAGTTCAACAGCACCTACAGGGTGGTGAGCGTGCTGACCGTGCTGCA
CCAGGACTGGCTGAACGGCAAGGAGTACAAGTGCAAGGTGAGCAACAAGGGCCTGCCC
AGCAGCATCGAGAAGACCATCAGCAAGGCCAAGGGCCAGCCCAGGGAGCCCCAGGTGT
ACACCCTGCCCCCCAGCCAGGAGGAGATGACCAAGAACCAGGTGAGCCTGACCTGCCTG
GTGAAGGGCTTCTACCCCAGCGACATCGCCGTGGAGTGGGAGAGCAACGGCCAGCCCGA
GAACAACTACAAGACCACCCCCCCCGTGCTGGACAGCGACGGCAGCTTCTTCCTGTACA
GCAGGCTGACCGTGGACAAGAGCAGGTGGCAGGAGGGCAACGTGTTCAGCTGCAGCGTG
ATGCACGAGGCCCTGCACAACCACTACACCCAGAAGAGCCTGAGCCTGAGCCTGGGCAA
GATGGCCCTGATCGTGCTGGGCGGCGTGGCCGGCCTGCTGCTGTTCATCGGCCTGGGCAT
CTTCTTCAGGGTGAAGTTCAGCAGGAGCGCCGACGCCCCCGCCTACCAGCAGGGCCAGA
ACCAGCTGTACAACGAGCTGAACCTGGGCAGGAGGGAGGAGTACGACGTGCTGGACAA
GAGGAGGGGCAGGGACCCCGAGATGGGCGGCAAGCCCAGGAGGAAGAACCCCCAGGAG
GGCCTGTACAACGAGCTGCAGAAGGACAAGATGGCCGAGGCCTACAGCGAGATCGGCAT
GAAGGGCGAGAGGAGGAGGGGCAAGGGCCACGACGGCCTGTACCAGGGCCTGAGCACC
GCCACCAAGGACACCTACGACGCCCTGCACATGCAGGCCCTGCCCCCCAGG

In some embodiments, a CAR construct as shown in SEQ ID NO: 22 is included in a vector. In some embodiments the recombinant expression vector includes a promoter (e.g., an SFFV promoter or an EF1α promoter). In some embodiments, the CAR may further comprise a signal peptide, such as the exemplary signal peptide provided by SEQ ID NO: 19.

### 2. IL3-WT-CD28costim

An exemplary CAR construct, as described herein, comprises a CD123-binding fragment of IL-3, a IgG4 hinge domain, a CD4 transmembrane domain, a CD28 costimulatory signaling domain, and a CD3ζ intracellular signaling domain. In some embodiments, the CAR may further comprise a signal peptide, such as the exemplary signal peptide provided by SEQ ID NO: 19.

In some embodiments, a CAR comprises an amino acid sequence shown in SEQ ID NO: 23, or an amino acid sequence that is at least 70%, at least 75%, at least 80%, at least 85%, at least 90%, at least 95%, or at least 99% identical to the amino acid sequence shown in SEQ ID NO: 23.

Amino acid sequence of IL3-WT-CD28costim
(SEQ ID NO: 23)
VNCSNMIDEIITHLKQPPLPLLDFNNLNGEDQDILMENNLRRPNLEAFNRAVKSLQNASAIESI
LKNLLPCLPLATAAPTRHPIHIKDGDWNEFRRKLTFYLKTLEESKYGPPCPPCPAPEFLGGPSV
FLFPPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQVS
LTCLVKGFYPSDIAVEWESNGQPENNYKTTPPVLDSDGSFFLYSRLTVDKSRWQEGNVFSCS
VMHEALHNHYTQKSLSLSLGKMALIVLGGVAGLLLFIGLGIFFRSKRSRLLHSDYMNMTPRR
PGPTRKHQYPYAPPRDFAAYRSRVKFSRSADAPAYQQGQNQLYNELNLGRREEYDVLDKRR
GRDPEMGGKPRRKNPQEGLYNELQKDKMAEAYSEIGMKGERRRGKGHDGLYQGLSTATKD
TYDALHMQALPPR

In some embodiments, a CAR is encoded by a nucleic acid sequence that comprises the sequence that is shown in SEQ ID NO: 24, or in a nucleic acid sequence that is at least 70%, at least 75%, at least 80%, at least 85%, at least 90%, at least 95%, or at least 99% identical to the nucleic acid sequence shown in SEQ ID NO: 24.

Nucleic acid sequence of IL3-WT-CD28costim
(SEQ ID NO: 24)
GTGAACTGCAGCAACATGATCGACGAGATCATCACCCACCTGAAGCAGCCCCCCCTGCC
CCTGCTGGACTTCAACAACCTGAACGGCGAGGACCAGGACATCCTGATGGAGAACAACC
TGAGGAGGCCCAACCTGGAGGCCTTCAACAGGGCCGTGAAGAGCCTGCAGAACGCCAGC
GCCATCGAGAGCATCCTGAAGAACCTGCTGCCCTGCCTGCCCCTGGCCACCGCCGCCCCC
ACCAGGCACCCCATCCACATCAAGGACGGCGACTGGAACGAGTTCAGGAGGAAGCTGAC
CTTCTACCTGAAGACCCTGGAGGAGAGCAAGTACGGCCCCCCCTGCCCCCCCTGCCCCGC
CCCCGAGTTCCTGGGCGGCCCCAGCGTGTTCCTGTTCCCCCCCAAGCCCAAGGACACCCT
GATGATCAGCAGGACCCCCGAGGTGACCTGCGTGGTGGTGGACGTGAGCCAGGAGGACC
CCGAGGTGCAGTTCAACTGGTACGTGGACGGCGTGGAGGTGCACAACGCCAAGACCAAG
CCCAGGGAGGAGCAGTTCAACAGCACCTACAGGGTGGTGAGCGTGCTGACCGTGCTGCA
CCAGGACTGGCTGAACGGCAAGGAGTACAAGTGCAAGGTGAGCAACAAGGGCCTGCCC
AGCAGCATCGAGAAGACCATCAGCAAGGCCAAGGGCCAGCCCAGGGAGCCCCAGGTGT
ACACCCTGCCCCCCAGCCAGGAGGAGATGACCAAGAACCAGGTGAGCCTGACCTGCCTG
GTGAAGGGCTTCTACCCCAGCGACATCGCCGTGGAGTGGGAGAGCAACGGCCAGCCCGA
GAACAACTACAAGACCACCCCCCCCGTGCTGGACAGCGACGGCAGCTTCTTCCTGTACA
GCAGGCTGACCGTGGACAAGAGCAGGTGGCAGGAGGGCAACGTGTTCAGCTGCAGCGTG
ATGCACGAGGCCCTGCACAACCACTACACCCAGAAGAGCCTGAGCCTGAGCCTGGGCAA
GATGGCCCTGATCGTGCTGGGCGGCGTGGCCGGCCTGCTGCTGTTCATCGGCCTGGGCAT
CTTCTTCAGGAGCAAGAGGAGCAGGCTGCTGCACAGCGACTACATGAACATGACCCCCA
GGAGGCCCGGCCCCACCAGGAAGCACCAGTACCCCTACGCCCCCCCCAGGGACTTCGCC
GCCTACAGGAGCAGGGTGAAGTTCAGCAGGAGCGCCGACGCCCCCGCCTACCAGCAGGG
CCAGAACCAGCTGTACAACGAGCTGAACCTGGGCAGGAGGGAGGAGTACGACGTGCTG
GACAAGAGGAGGGGCAGGGACCCCGAGATGGGCGGCAAGCCCAGGAGGAAGAACCCCC
AGGAGGGCCTGTACAACGAGCTGCAGAAGGACAAGATGGCCGAGGCCTACAGCGAGAT
CGGCATGAAGGGCGAGAGGAGGAGGGGCAAGGGCCACGACGGCCTGTACCAGGGCCTG
AGCACCGCCACCAAGGACACCTACGACGCCCTGCACATGCAGGCCCTGCCCCCCAGG

In some embodiments, a CAR construct as shown in SEQ ID NO: 24 is included in a vector. In some embodiments the recombinant expression vector includes a promoter (e.g., an SFFV promoter or an EF1α promoter).

### 3. IL3-WT-41BBcostim

An exemplary CAR construct, as described herein, comprises a CD123-binding fragment of IL-3, a IgG4 hinge domain, a CD4 transmembrane domain, a 4-1BB costimulatory signaling domain, and a CD3ζ intracellular signaling domain. In some embodiments, the CAR may further comprise a signal peptide, such as the exemplary signal peptide provided by SEQ ID NO: 19.

In some embodiments, a CAR comprises an amino acid sequence shown in SEQ ID NO: 25, or an amino acid sequence that is at least 70%, at least 75%, at least 80%, at least 85%, at least 90%, at least 95%, or at least 99% identical to the amino acid sequence shown in SEQ ID NO: 25.

Amino acid sequence of IL3-WT-4-1BBcostim
(SEQ ID NO: 25)
VNCSNMIDEIITHLKQPPLPLLDFNNLNGEDQDILMENNLRRPNLEAFN
RAVKSLQNASAIESILKNLLPCLPLATAAPTRHPIHIKDGDWNEFRRKL
TFYLKTLEESKYGPPCPPCPAPEFLGGPSVFLFPPKPKDTLMISRTPEV
TCVVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFL
YSRLTVDKSRWQEGNVFSCSVMHEALHNHYTQKSLSLSLGKMALIVLGG
VAGLLLFIGLGIFFKRGRKKLLYIFKQPFMRPVQTTQEEDGCSCRFPEE
EEGGCELRVKFSRSADAPAYQQGQNQLYNELNLGRREEYDVLDKRRGRD
PEMGGKPRRKNPQEGLYNELQKDKMAEAYSEIGMKGERRRGKGHDGLYQ
GLSTATKDTYDALHMQALPPR

In some embodiments, a CAR is encoded by a nucleic acid sequence that comprises the sequence that is shown in SEQ ID NO: 26, or in a nucleic acid sequence that is at least 70%, at least 75%, at least 80%, at least 85%, at least 90%, at least 95%, or at least 99% identical to the nucleic acid sequence shown in SEQ ID NO: 26.

Nucleic acid sequence of IL3-WT-4-1BBcostim
(SEQ ID NO: 26)
GTGAACTGCAGCAACATGATCGACGAGATCATCACCCACCTGAAGCAGCCCCCCCTGCC
CCTGCTGGACTTCAACAACCTGAACGGCGAGGACCAGGACATCCTGATGGAGAACAACC
TGAGGAGGCCCAACCTGGAGGCCTTCAACAGGGCCGTGAAGAGCCTGCAGAACGCCAGC
GCCATCGAGAGCATCCTGAAGAACCTGCTGCCCTGCCTGCCCCTGGCCACCGCCGCCCCC
ACCAGGCACCCCATCCACATCAAGGACGGCGACTGGAACGAGTTCAGGAGGAAGCTGAC
CTTCTACCTGAAGACCCTGGAGGAGAGCAAGTACGGCCCCCCCTGCCCCCCCTGCCCCGC
CCCCGAGTTCCTGGGCGGCCCCAGCGTGTTCCTGTTCCCCCCCAAGCCCAAGGACACCCT
GATGATCAGCAGGACCCCCGAGGTGACCTGCGTGGTGGTGGACGTGAGCCAGGAGGACC
CCGAGGTGCAGTTCAACTGGTACGTGGACGGCGTGGAGGTGCACAACGCCAAGACCAAG
CCCAGGGAGGAGCAGTTCAACAGCACCTACAGGGTGGTGAGCGTGCTGACCGTGCTGCA
CCAGGACTGGCTGAACGGCAAGGAGTACAAGTGCAAGGTGAGCAACAAGGGCCTGCCC
AGCAGCATCGAGAAGACCATCAGCAAGGCCAAGGGCCAGCCCAGGGAGCCCCAGGTGT
ACACCCTGCCCCCCAGCCAGGAGGAGATGACCAAGAACCAGGTGAGCCTGACCTGCCTG
GTGAAGGGCTTCTACCCCAGCGACATCGCCGTGGAGTGGGAGAGCAACGGCCAGCCCGA
GAACAACTACAAGACCACCCCCCCCGTGCTGGACAGCGACGGCAGCTTCTTCCTGTACA
GCAGGCTGACCGTGGACAAGAGCAGGTGGCAGGAGGGCAACGTGTTCAGCTGCAGCGTG
ATGCACGAGGCCCTGCACAACCACTACACCCAGAAGAGCCTGAGCCTGAGCCTGGGCAA
GATGGCCCTGATCGTGCTGGGCGGCGTGGCCGGCCTGCTGCTGTTCATCGGCCTGGGCAT
CTTCTTCAAGAGGGGCAGGAAGAAGCTGCTGTACATCTTCAAGCAGCCCTTCATGAGGCC
CGTGCAGACCACCCAGGAGGAGGACGGCTGCAGCTGCAGGTTCCCCGAGGAGGAGGAG
GGCGGCTGCGAGCTGAGGGTGAAGTTCAGCAGGAGCGCCGACGCCCCCGCCTACCAGCA
GGGCCAGAACCAGCTGTACAACGAGCTGAACCTGGGCAGGAGGGAGGAGTACGACGTG
CTGGACAAGAGGAGGGGCAGGGACCCCGAGATGGGCGGCAAGCCCAGGAGGAAGAACC
CCCAGGAGGGCCTGTACAACGAGCTGCAGAAGGACAAGATGGCCGAGGCCTACAGCGA
GATCGGCATGAAGGGCGAGAGGAGGAGGGGCAAGGGCCACGACGGCCTGTACCAGGGC
CTGAGCACCGCCACCAAGGACACCTACGACGCCCTGCACATGCAGGCCCTGCCCCCCAG
G

In some embodiments, a CAR construct as shown in SEQ ID NO: 26 is included in a vector. In some embodiments the recombinant expression vector includes a promoter (e.g., an SFFV promoter or an EF1α promoter).

### 4. IL3-K110E-Nocostim

An exemplary CAR construct, as described herein, comprises a CD123-binding fragment of IL-3 containing a K110E mutation, a IgG4 hinge domain, a CD4 transmembrane domain, and a CD3ζ intracellular signaling domain. In some embodiments, the CAR may further comprise a signal peptide, such as the exemplary signal peptide provided by SEQ ID NO: 19.

In some embodiments, a CAR comprises an amino acid sequence shown in SEQ ID NO: 27, or an amino acid sequence that is at least 70%, at least 75%, at least 80%, at least 85%, at least 90%, at least 95%, or at least 99% identical to the amino acid sequence shown in SEQ ID NO: 27.

Amino acid sequence of IL3-K110E nocostim
(SEQ ID NO: 27)
VNCSNMIDEIITHLKQPPLPLLDFNNLNGEDQDILMENNLRRPNLEAFN
RAVKSLQNASAIESILKNLLPCLPLATAAPTRHPIHIKDGDWNEFRREL
TFYLKTLEESKYGPPCPPCPAPEFLGGPSVFLFPPKPKDTLMISRTPEV
TCVVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFL
YSRLTVDKSRWQEGNVFSCSVMHEALHNHYTQKSLSLSLGKMALIVLGG
VAGLLLFIGLGIFFRVKFSRSADAPAYQQGQNQLYNELNLGRREEYDVL
DKRRGRDPEMGGKPRRKNPQEGLYNELQKDKMAEAYSEIGMKGERRRGK
GHDGLYQGLSTATKDTYDALHMQALPPR

In some embodiments, a CAR in encoded by a nucleic acid sequence that comprises the sequence that is shown in SEQ ID NO: 28, or in a nucleic acid sequence that is at least 70%, at least 75%, at least 80%, at least 85%, at least 90%, at least 95%, or at least 99% identical to the nucleic acid sequence shown in SEQ ID NO: 28.

Nucleic acid sequence of IL3-K110E-nocostim
(SEQ ID NO: 28)
GTGAACTGCAGCAACATGATCGACGAGATCATCACCCACCTGAAGCAGC
CCCCCCTGCCCCTGCTGGACTTCAACAACCTGAACGGCGAGGACCAGGA
CATCCTGATGGAGAACAACCTGAGGAGGCCCAACCTGGAGGCCTTCAAC
AGGGCCGTGAAGAGCCTGCAGAACGCCAGCGCCATCGAGAGCATCCTGA
AGAACCTGCTGCCCTGCCTGCCCCTGGCCACCGCCGCCCCCACCAGGCA
CCCCATCCACATCAAGGACGGCGACTGGAACGAGTTCAGGAGGGAGCTG
ACCTTCTACCTGAAGACCCTGGAGGAGAGCAAGTACGGCCCCCCCTGCC
CCCCCTGCCCCGCCCCCGAGTTCCTGGGCGGCCCCAGCGTGTTCCTGTT
CCCCCCCAAGCCCAAGGACACCCTGATGATCAGCAGGACCCCCGAGGTG
ACCTGCGTGGTGGTGGACGTGAGCCAGGAGGACCCCGAGGTGCAGTTCA
ACTGGTACGTGGACGGCGTGGAGGTGCACAACGCCAAGACCAAGCCCAG
GGAGGAGCAGTTCAACAGCACCTACAGGGTGGTGAGCGTGCTGACCGTG
CTGCACCAGGACTGGCTGAACGGCAAGGAGTACAAGTGCAAGGTGAGCA
ACAAGGGCCTGCCCAGCAGCATCGAGAAGACCATCAGCAAGGCCAAGGG
CCAGCCCAGGGAGCCCCAGGTGTACACCCTGCCCCCCAGCCAGGAGGAG
ATGACCAAGAACCAGGTGAGCCTGACCTGCCTGGTGAAGGGCTTCTACC
CCAGCGACATCGCCGTGGAGTGGGAGAGCAACGGCCAGCCCGAGAACAA
CTACAAGACCACCCCCCCCGTGCTGGACAGCGACGGCAGCTTCTTCCTG
TACAGCAGGCTGACCGTGGACAAGAGCAGGTGGCAGGAGGGCAACGTGT
TCAGCTGCAGCGTGATGCACGAGGCCCTGCACAACCACTACACCCAGAA
GAGCCTGAGCCTGAGCCTGGGCAAGATGGCCCTGATCGTGCTGGGCGGC
GTGGCCGGCCTGCTGCTGTTCATCGGCCTGGGCATCTTCTTCAGGGTGA
AGTTCAGCAGGAGCGCCGACGCCCCCGCCTACCAGCAGGGCCAGAACCA
GCTGTACAACGAGCTGAACCTGGGCAGGAGGGAGGAGTACGACGTGCTG
GACAAGAGGAGGGGCAGGGACCCCGAGATGGGCGGCAAGCCCAGGAGGA
AGAACCCCCAGGAGGGCCTGTACAACGAGCTGCAGAAGGACAAGATGGC
CGAGGCCTACAGCGAGATCGGCATGAAGGGCGAGAGGAGGAGGGGCAAG
GGCCACGACGGCCTGTACCAGGGCCTGAGCACCGCCACCAAGGACACCT
ACGACGCCCTGCACATGCAGGCCCTGCCCCCCAGG

In some embodiments, a CAR construct as shown in SEQ ID NO: 28 is included in a vector. In some embodiments the recombinant expression vector includes a promoter (e.g., an SFFV promoter or an EF1α promoter).

### 5. IL3-K110E-CD28costim

An exemplary CAR construct, as described herein, comprises a CD123-binding fragment of IL-3 containing a K110E mutation, a IgG4 hinge domain, a CD4 transmembrane domain, a CD28 costimulatory signaling domain, and a CD3 intracellular signaling domain. In some embodiments, the CAR may further comprise a signal peptide, such as the exemplary signal peptide provided by SEQ ID NO: 19.

In some embodiments, a CAR comprises an amino acid sequence shown in SEQ ID NO: 29, or an amino acid sequence that is at least 70%, at least 75%, at least 80%, at least 85%, at least 90%, at least 95%, or at least 99% identical to the amino acid sequence shown in SEQ ID NO: 29.

Amino acid sequence of IL3-K110E CD28costim
(SEQ ID NO: 29)
VNCSNMIDEIITHLKQPPLPLLDFNNLNGEDQDILMENNLRRPNLEAFN
RAVKSLQNASAIESILKNLLPCLPLATAAPTRHPIHIKDGDWNEFRREL
TFYLKTLEESKYGPPCPPCPAPEFLGGPSVFLFPPKPKDTLMISRTPEV
TCVVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFL
YSRLTVDKSRWQEGNVFSCSVMHEALHNHYTQKSLSLSLGKMALIVLGG
VAGLLLFIGLGIFFRSKRSRLLHSDYMNMTPRRPGPTRKHQYPYAPPRD
FAAYRSRVKFSRSADAPAYQQGQNQLYNELNLGRREEYDVLDKRRGRDP
EMGGKPRRKNPQEGLYNELQKDKMAEAYSEIGMKGERRRGKGHDGLYQG
LSTATKDTYDALHMQALPPR

In some embodiments, a CAR in encoded by a nucleic acid sequence that comprises the sequence that is shown in SEQ ID NO: 30, or in a nucleic acid sequence that is at least 70%, at least 75%, at least 80%, at least 85%, at least 90%, at least 95%, or at least 99% identical to the nucleic acid sequence shown in SEQ ID NO: 30.

Nucleic acid sequence of IL3-K110E-CD28costim
(SEQ ID NO: 30)
GTGAACTGCAGCAACATGATCGACGAGATCATCACCCACCTGAAGCAGC
CCCCCCTGCCCCTGCTGGACTTCAACAACCTGAACGGCGAGGACCAGGA
CATCCTGATGGAGAACAACCTGAGGAGGCCCAACCTGGAGGCCTTCAAC
AGGGCCGTGAAGAGCCTGCAGAACGCCAGCGCCATCGAGAGCATCCTGA
AGAACCTGCTGCCCTGCCTGCCCCTGGCCACCGCCGCCCCCACCAGGCA
CCCCATCCACATCAAGGACGGCGACTGGAACGAGTTCAGGAGGGAGCTG
ACCTTCTACCTGAAGACCCTGGAGGAGAGCAAGTACGGCCCCCCCTGCC
CCCCCTGCCCCGCCCCCGAGTTCCTGGGCGGCCCCAGCGTGTTCCTGTT
CCCCCCCAAGCCCAAGGACACCCTGATGATCAGCAGGACCCCCGAGGTG
ACCTGCGTGGTGGTGGACGTGAGCCAGGAGGACCCCGAGGTGCAGTTCA
ACTGGTACGTGGACGGCGTGGAGGTGCACAACGCCAAGACCAAGCCCAG
GGAGGAGCAGTTCAACAGCACCTACAGGGTGGTGAGCGTGCTGACCGTG
CTGCACCAGGACTGGCTGAACGGCAAGGAGTACAAGTGCAAGGTGAGCA
ACAAGGGCCTGCCCAGCAGCATCGAGAAGACCATCAGCAAGGCCAAGGG
CCAGCCCAGGGAGCCCCAGGTGTACACCCTGCCCCCCAGCCAGGAGGAG
ATGACCAAGAACCAGGTGAGCCTGACCTGCCTGGTGAAGGGCTTCTACC
CCAGCGACATCGCCGTGGAGTGGGAGAGCAACGGCCAGCCCGAGAACAA
CTACAAGACCACCCCCCCCGTGCTGGACAGCGACGGCAGCTTCTTCCTG
TACAGCAGGCTGACCGTGGACAAGAGCAGGTGGCAGGAGGGCAACGTGT
TCAGCTGCAGCGTGATGCACGAGGCCCTGCACAACCACTACACCCAGAA
GAGCCTGAGCCTGAGCCTGGGCAAGATGGCCCTGATCGTGCTGGGCGGC
GTGGCCGGCCTGCTGCTGTTCATCGGCCTGGGCATCTTCTTCAGGAGCA
AGAGGAGCAGGCTGCTGCACAGCGACTACATGAACATGACCCCCAGGAG
GCCCGGCCCCACCAGGAAGCACCAGTACCCCTACGCCCCCCCCAGGGAC
TTCGCCGCCTACAGGAGCAGGGTGAAGTTCAGCAGGAGCGCCGACGCCC
CCGCCTACCAGCAGGGCCAGAACCAGCTGTACAACGAGCTGAACCTGGG
CAGGAGGGAGGAGTACGACGTGCTGGACAAGAGGAGGGGCAGGGACCCC
GAGATGGGCGGCAAGCCCAGGAGGAAGAACCCCCAGGAGGGCCTGTACA
ACGAGCTGCAGAAGGACAAGATGGCCGAGGCCTACAGCGAGATCGGCAT
GAAGGGCGAGAGGAGGAGGGGCAAGGGCCACGACGGCCTGTACCAGGGC
CTGAGCACCGCCACCAAGGACACCTACGACGCCCTGCACATGCAGGCCC
TGCCCCCCAGG

In some embodiments, a CAR construct as shown in SEQ ID NO: 30 is included in a vector. In some embodiments the recombinant expression vector includes a promoter (e.g., an SFFV promoter or an EF1α promoter).

### 6. IL3-K110E-41BBcostim

An exemplary CAR construct, as described herein, comprises a CD123-binding fragment of IL-3 containing a K110E mutation, a IgG4 hinge domain, a CD4 transmembrane domain, a 4-1BB costimulatory signaling domain, and a CD3ζ intracellular signaling domain. In some embodiments, the CAR may further comprise a signal peptide, such as the exemplary signal peptide provided by SEQ ID NO: 19.

In some embodiments, a CAR comprises an amino acid sequence shown in SEQ ID NO: 31, or an amino acid sequence that is at least 70%, at least 75%, at least 80%, at least 85%, at least 90%, at least 95%, or at least 99% identical to the amino acid sequence shown in SEQ ID NO: 31.

Amino acid sequence of IL3-K110E 4-1BBcostim
(SEQ ID NO: 31)
VNCSNMIDEIITHLKQPPLPLLDFNNLNGEDQDILMENNLRRPNLEAFN
RAVKSLQNASAIESILKNLLPCLPLATAAPTRHPIHIKDGDWNEFRREL
TFYLKTLEESKYGPPCPPCPAPEFLGGPSVFLFPPKPKDTLMISRTPEV
TCVVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFL
YSRLTVDKSRWQEGNVFSCSVMHEALHNHYTQKSLSLSLGKMALIVLGG
VAGLLLFIGLGIFFKRGRKKLLYIFKQPFMRPVQTTQEEDGCSCRFPEE
EEGGCELRVKFSRSADAPAYQQGQNQLYNELNLGRREEYDVLDKRRGRD
PEMGGKPRRKNPQEGLYNELQKDKMAEAYSEIGMKGERRRGKGHDGLYQ
GLSTATKDTYDALHMQALPPR

In some embodiments, a CAR in encoded by a nucleic acid sequence that comprises the sequence that is shown in SEQ ID NO: 32, or in a nucleic acid sequence that is at least 70%, at least 75%, at least 80%, at least 85%, at least 90%, at least 95%, or at least 99% identical to the nucleic acid sequence shown in SEQ ID NO: 32.

Nucleic acid sequence of IL3-K110E-4-1BBcostim
(SEQ ID NO: 32)
GTGAACTGCAGCAACATGATCGACGAGATCATCACCCACCTGAAGCAGCCCCCCCTGCC
CCTGCTGGACTTCAACAACCTGAACGGCGAGGACCAGGACATCCTGATGGAGAACAACC
TGAGGAGGCCCAACCTGGAGGCCTTCAACAGGGCCGTGAAGAGCCTGCAGAACGCCAGC
GCCATCGAGAGCATCCTGAAGAACCTGCTGCCCTGCCTGCCCCTGGCCACCGCCGCCCCC
ACCAGGCACCCCATCCACATCAAGGACGGCGACTGGAACGAGTTCAGGAGGGAGCTGAC
CTTCTACCTGAAGACCCTGGAGGAGAGCAAGTACGGCCCCCCCTGCCCCCCCTGCCCCGC
CCCCGAGTTCCTGGGCGGCCCCAGCGTGTTCCTGTTCCCCCCCAAGCCCAAGGACACCCT
GATGATCAGCAGGACCCCCGAGGTGACCTGCGTGGTGGTGGACGTGAGCCAGGAGGACC
CCGAGGTGCAGTTCAACTGGTACGTGGACGGCGTGGAGGTGCACAACGCCAAGACCAAG
CCCAGGGAGGAGCAGTTCAACAGCACCTACAGGGTGGTGAGCGTGCTGACCGTGCTGCA
CCAGGACTGGCTGAACGGCAAGGAGTACAAGTGCAAGGTGAGCAACAAGGGCCTGCCC
AGCAGCATCGAGAAGACCATCAGCAAGGCCAAGGGCCAGCCCAGGGAGCCCCAGGTGT
ACACCCTGCCCCCCAGCCAGGAGGAGATGACCAAGAACCAGGTGAGCCTGACCTGCCTG
GTGAAGGGCTTCTACCCCAGCGACATCGCCGTGGAGTGGGAGAGCAACGGCCAGCCCGA
GAACAACTACAAGACCACCCCCCCCGTGCTGGACAGCGACGGCAGCTTCTTCCTGTACA
GCAGGCTGACCGTGGACAAGAGCAGGTGGCAGGAGGGCAACGTGTTCAGCTGCAGCGTG
ATGCACGAGGCCCTGCACAACCACTACACCCAGAAGAGCCTGAGCCTGAGCCTGGGCAA
GATGGCCCTGATCGTGCTGGGCGGCGTGGCCGGCCTGCTGCTGTTCATCGGCCTGGGCAT
CTTCTTCAAGAGGGGCAGGAAGAAGCTGCTGTACATCTTCAAGCAGCCCTTCATGAGGCC
CGTGCAGACCACCCAGGAGGAGGACGGCTGCAGCTGCAGGTTCCCCGAGGAGGAGGAG
GGCGGCTGCGAGCTGAGGGTGAAGTTCAGCAGGAGCGCCGACGCCCCCGCCTACCAGCA
GGGCCAGAACCAGCTGTACAACGAGCTGAACCTGGGCAGGAGGGAGGAGTACGACGTG
CTGGACAAGAGGAGGGGCAGGGACCCCGAGATGGGCGGCAAGCCCAGGAGGAAGAACC
CCCAGGAGGGCCTGTACAACGAGCTGCAGAAGGACAAGATGGCCGAGGCCTACAGCGA
GATCGGCATGAAGGGCGAGAGGAGGAGGGGCAAGGGCCACGACGGCCTGTACCAGGGC
CTGAGCACCGCCACCAAGGACACCTACGACGCCCTGCACATGCAGGCCCTGCCCCCCAG
G

In some embodiments, a CAR construct as shown in SEQ ID NO: 32 is included in a vector. In some embodiments the recombinant expression vector includes a promoter (e.g., an SFFV promoter or an EF1α promoter).

### 7. IL3-WT-Nocostim+CD8 Transmembrane

An exemplary CAR construct, as described herein, comprises a CD123-binding fragment, a IgG4 hinge domain, a CD8 transmembrane domain, and a CD3 intracellular signaling domain. In some embodiments, the CAR may further comprise a signal peptide, such as the exemplary signal peptide provided by SEQ ID NO: 19.

In some embodiments, a CAR comprises an amino acid sequence shown in SEQ ID NO: 33, or an amino acid sequence that is at least 70%, at least 75%, at least 80%, at least 85%, at least 90%, at least 95%, or at least 99% identical to the amino acid sequence shown in SEQ ID NO: 33.

Amino acid sequence of IL3-WT-nocostim + CD8 transmembrane
(SEQ ID NO: 33)
VNCSNMIDEIITHLKQPPLPLLDFNNLNGEDQDILMENNLRRPNLEAFNRAVKSLQNA
SAIESILKNLLPCLPLATAAPTRHPIHIKDGDWNEFRRKLTFYLKTLEESKYGPPCPPCP
APEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGKIYIWAPLAGTC
GVLLLSLVITLYCRVKFSRSADAPAYQQGQNQLYNELNLGRREEYDVLDKRRGRDP
EMGGKPRRKNPQEGLYNELQKDKMAEAYSEIGMKGERRRGKGHDGLYQGLSTATK
DTYDALHMQALPPR

In some embodiments, a CAR is encoded by a nucleic acid sequence that comprises the sequence that is shown in SEQ ID NO: 34, or in a nucleic acid sequence that is at least 70%, at least 75%, at least 80%, at least 85%, at least 90%, at least 95%, or at least 99% identical to the nucleic acid sequence shown in SEQ ID NO: 34.

Nucleic acid sequence of IL3-WT-nocostim + CD8 transmembrane
(SEQ ID NO: 34)
GTGAACTGCAGCAACATGATCGACGAGATCATCACCCACCTGAAGCAGCCCCCCCTGCC
CCTGCTGGACTTCAACAACCTGAACGGCGAGGACCAGGACATCCTGATGGAGAACAACC
TGAGGAGGCCCAACCTGGAGGCCTTCAACAGGGCCGTGAAGAGCCTGCAGAACGCCAGC
GCCATCGAGAGCATCCTGAAGAACCTGCTGCCCTGCCTGCCCCTGGCCACCGCCGCCCCC
ACCAGGCACCCCATCCACATCAAGGACGGCGACTGGAACGAGTTCAGGAGGAAGCTGAC
CTTCTACCTGAAGACCCTGGAGGAGAGCAAGTACGGCCCCCCCTGCCCCCCCTGCCCCGC
CCCCGAGTTCCTGGGCGGCCCCAGCGTGTTCCTGTTCCCCCCCAAGCCCAAGGACACCCT
GATGATCAGCAGGACCCCCGAGGTGACCTGCGTGGTGGTGGACGTGAGCCAGGAGGACC
CCGAGGTGCAGTTCAACTGGTACGTGGACGGCGTGGAGGTGCACAACGCCAAGACCAAG
CCCAGGGAGGAGCAGTTCAACAGCACCTACAGGGTGGTGAGCGTGCTGACCGTGCTGCA
CCAGGACTGGCTGAACGGCAAGGAGTACAAGTGCAAGGTGAGCAACAAGGGCCTGCCC
AGCAGCATCGAGAAGACCATCAGCAAGGCCAAGGGCCAGCCCAGGGAGCCCCAGGTGT
ACACCCTGCCCCCCAGCCAGGAGGAGATGACCAAGAACCAGGTGAGCCTGACCTGCCTG
GTGAAGGGCTTCTACCCCAGCGACATCGCCGTGGAGTGGGAGAGCAACGGCCAGCCCGA
GAACAACTACAAGACCACCCCCCCCGTGCTGGACAGCGACGGCAGCTTCTTCCTGTACA
GCAGGCTGACCGTGGACAAGAGCAGGTGGCAGGAGGGCAACGTGTTCAGCTGCAGCGTG
ATGCACGAGGCCCTGCACAACCACTACACCCAGAAGAGCCTGAGCCTGAGCCTGGGCAA
GATCTACATCTGGGCCCCCCTGGCCGGCACCTGCGGCGTGCTGCTGCTGAGCCTGGTGAT
CACCCTGTACTGCAGGGTGAAGTTCAGCAGGAGCGCCGACGCCCCCGCCTACCAGCAGG
GCCAGAACCAGCTGTACAACGAGCTGAACCTGGGCAGGAGGGAGGAGTACGACGTGCT
GGACAAGAGGAGGGGCAGGGACCCCGAGATGGGCGGCAAGCCCAGGAGGAAGAACCCC
CAGGAGGGCCTGTACAACGAGCTGCAGAAGGACAAGATGGCCGAGGCCTACAGCGAGA
TCGGCATGAAGGGCGAGAGGAGGAGGGGCAAGGGCCACGACGGCCTGTACCAGGGCCT
GAGCACCGCCACCAAGGACACCTACGACGCCCTGCACATGCAGGCCCTGCCCCCCAGG

In some embodiments, a CAR construct as shown in SEQ ID NO: 34 is included in a vector. In some embodiments the recombinant expression vector includes a promoter (e.g., an SFFV promoter or an EF1α promoter).

### 8. IL3-WT-Nocostim+IgG4 L235E, N297Qhinge

An exemplary CAR construct, as described herein, comprises a CD123-binding fragment, a IgG4 hinge domain containing L235E and N297Q mutations, a CD4 transmembrane domain, and a CD3ζ intracellular signaling domain. In some embodiments, the CAR may further comprise a signal peptide, such as the exemplary signal peptide provided by SEQ ID NO: 19.

In some embodiments, a CAR comprises an amino acid sequence shown in SEQ ID NO: 35, or an amino acid sequence that is at least 70%, at least 75%, at least 80%, at least 85%, at least 90%, at least 95%, or at least 99% identical to the amino acid sequence shown in SEQ ID NO: 35.

Amino acid sequence of IL3-WT-nocostim + IgG4 L235E, N297hinge
(SEQ ID NO: 35)
VNCSNMIDEIITHLKQPPLPLLDENNLNGEDQDILMENNLRRPNLEAFNRAVKSLQNA
SAIESILKNLLPCLPLATAAPTRHPIHIKDGDWNEFRRKLTFYLKTLEESKYGPPCPSCP
APEFEGGPSVFLPPKPKDTLMISRTPEVTCVVVDVSQEDPEVQFNWYVDGVEVHQA
KTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGKMALIVLGGVA
GLLLFIGLGIFFRVKFSRSADAPAYQQGQNQLYNELNLGRREEYDVLDKRRGRDPEM
GGKPRRKNPQEGLYNELQKDKMAEAYSEIGMKGERRRGKGHDGLYQGLSTATKDT
YDALHMQALPPR

In some embodiments, a CAR is encoded by a nucleic acid sequence that comprises the sequence that is shown in SEQ ID NO: 36, or in a nucleic acid sequence that is at least 70%, at least 75%, at least 80%, at least 85%, at least 90%, at least 95%, or at least 99% identical to the nucleic acid sequence shown in SEQ ID NO: 36.

Nucleic acid sequence of IL3-WT-nocostim + IgG4 L235E, N297hinge
(SEQ ID NO: 36)
GTGAACTGCAGCAACATGATCGACGAGATCATCACCCACCTGAAGCAGCCCCCCCTGCC
CCTGCTGGACTTCAACAACCTGAACGGCGAGGACCAGGACATCCTGATGGAGAACAACC
TGAGGAGGCCCAACCTGGAGGCCTTCAACAGGGCCGTGAAGAGCCTGCAGAACGCCAGC
GCCATCGAGAGCATCCTGAAGAACCTGCTGCCCTGCCTGCCCCTGGCCACCGCCGCCCCC
ACCAGGCACCCCATCCACATCAAGGACGGCGACTGGAACGAGTTCAGGAGGAAGCTGAC
CTTCTACCTGAAGACCCTGGAGGAGAGCAAGTACGGCCCCCCCTGCCCCAGCTGCCCCGC
CCCCGAGTTCGAGGGCGGCCCCAGCGTGTTCCTGCCCCCCAAGCCCAAGGACACCCTGAT
GATCAGCAGGACCCCCGAGGTGACCTGCGTGGTGGTGGACGTGAGCCAGGAGGACCCCG
AGGTGNNNTTCAACTGGTACGTGGACGGCGTGGAGGTGCACNNNGCCAAGACCAAGCCC
AGGGAGGAGCAGTTCAACAGCACCTACAGGGTGGTGAGCGTGCTGACCGTGCTGCACNN
NGACTGGCTGAACGGCAAGGAGTACAAGTGCAAGGTGAGCAACAAGGGCCTGCCCAGC
AGCATCGAGAAGACCATCAGCAAGGCCAAGGGCCAGCCCAGGGAGCCCNNNGTGTACA
CCCTGCCCCCCAGCNNNGAGGAGATGACCAAGAACNNNGTGAGCCTGACCTGCCTGGTG
AAGGGCTTCTACCCCAGCGACATCGCCGTGGAGTGGGAGAGCAACGGCNNNCCCGAGAA
CAACTACAAGACCACCCCCCCCGTGCTGGACAGCGACGGCAGCTTCTTCCTGTACAGCAG
GCTGACCGTGGACAAGAGCAGGTGGCAGGAGGGCAACGTGTTCAGCTGCAGCGTGATGC
ACGAGGCCCTGCACAACCACTACACCNNNAAGAGCCTGAGCCTGAGCCTGGGCAAGATG
GCCCTGATCGTGCTGGGCGGCGTGGCCGGCCTGCTGCTGTTCATCGGCCTGGGCATCTTC
TTCAGGGTGAAGTTCAGCAGGAGCGCCGACGCCCCCGCCTACCAGCAGGGCCAGAACCA
GCTGTACAACGAGCTGAACCTGGGCAGGAGGGAGGAGTACGACGTGCTGGACAAGAGG
AGGGGCAGGGACCCCGAGATGGGCGGCAAGCCCAGGAGGAAGAACCCCCAGGAGGGCC
TGTACAACGAGCTGCAGAAGGACAAGATGGCCGAGGCCTACAGCGAGATCGGCATGAA
GGGCGAGAGGAGGAGGGGCAAGGGCCACGACGGCCTGTACCAGGGCCTGAGCACCGCC
ACCAAGGACACCTACGACGCCCTGCACATGCAGGCCCTGCCCCCCAGG

In some embodiments, a CAR construct as shown in SEQ ID NO: 36 is included in a vector. In some embodiments the recombinant expression vector includes a promoter (e.g., an SFFV promoter or an EF1α promoter).

### 9. IL3-WT-Nocostim+CD8 Hinge

An exemplary CAR construct, as described herein, comprises a CD123-binding fragment, a CD8 hinge domain, a CD4 transmembrane domain, and a CD3ζ intracellular signaling domain. In some embodiments, the CAR may further comprise a signal peptide, such as the exemplary signal peptide provided by SEQ ID NO: 19

In some embodiments, a CAR comprises an amino acid sequence shown in SEQ ID NO: 37 or an amino acid sequence that is at least 70%, at least 75%, at least 80%, at least 85%, at least 90%, at least 95%, or at least 99% identical to the amino acid sequence shown in SEQ ID NO: 37

Amino acid sequence of IL3-WT-nocostim + CD8 hinge
(SEQ ID NO: 37)
VNCSNMIDEIITHLKQPPLPLLDFNNLNGEDQDILMENNLRRPNLEAFNRAVKSLQNA
SAIESILKNLLPCLPLATAAPTRHPIHIKDGDWNEFRRKLTFYLKTLETTTPAPRPPTPA
PTIASQPLSLRPEACRPAAGGAVHTRGLDFACDMALIVLGGVAGLLLFIGLGIFFRVK
FSRSADAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR

In some embodiments, a CAR is encoded by a nucleic acid sequence that comprises the sequence that is shown in SEQ ID NO: 38, or in a nucleic acid sequence that is at least 70%, at least 75%, at least 80%, at least 85%, at least 90%, at least 95%, or at least 99% identical to the nucleic acid sequence shown in SEQ ID NO: 38.

Nucleic acid sequence of IL3-WT-nocostim + CD8 hinge
(SEQ ID NO: 38)
GTGAACTGCAGCAACATGATCGACGAGATCATCACCCACCTGAAGCAGCCCCCCCTGCC
CCTGCTGGACTTCAACAACCTGAACGGCGAGGACCAGGACATCCTGATGGAGAACAACC
TGAGGAGGCCCAACCTGGAGGCCTTCAACAGGGCCGTGAAGAGCCTGCAGAACGCCAGC
GCCATCGAGAGCATCCTGAAGAACCTGCTGCCCTGCCTGCCCCTGGCCACCGCCGCCCCC
ACCAGGCACCCCATCCACATCAAGGACGGCGACTGGAACGAGTTCAGGAGGAAGCTGAC
CTTCTACCTGAAGACCCTGGAGACCACCACCCCCGCCCCCAGGCCCCCCACCCCCGCCCC
CACCATCGCCAGCNNNCCCCTGAGCCTGAGGCCCGAGGCCTGCAGGCCCGCCGCCGGCG
GCGCCGTGCACACCAGGGGCCTGGACTTCGCCTGCGACATGGCCCTGATCGTGCTGGGC
GGCGTGGCCGGCCTGCTGCTGTTCATCGGCCTGGGCATCTTCTTCAGGGTGAAGTTCAGC
AGGAGCGCCGACGCCCCCGCCTACCAGCAGGGCCAGAACCAGCTGTACAACGAGCTGAA
CCTGGGCAGGAGGGAGGAGTACGACGTGCTGGACAAGAGGAGGGGCAGGGACCCCGAG
ATGGGCGGCAAGCCCAGGAGGAAGAACCCCCAGGAGGGCCTGTACAACGAGCTGCAGA
AGGACAAGATGGCCGAGGCCTACAGCGAGATCGGCATGAAGGGCGAGAGGAGGAGGGG
CAAGGGCCACGACGGCCTGTACCAGGGCCTGAGCACCGCCACCAAGGACACCTACGACG
CCCTGCACATGCAGGCCCTGCCCCCCAGG

In some embodiments, a CAR construct as shown in SEQ ID NO: 38 is included in a vector. In some embodiments the recombinant expression vector includes a promoter (e.g., an SFFV promoter or an EF1α promoter).

### 10. IL3-D110A, K116V-Nocostim

An exemplary CAR construct, as described herein, comprises a CD123-binding fragment containing D101A and K116V mutations, a IgG4 hinge domain, a CD4 transmembrane domain, and a CD3ζ intracellular signaling domain. In some embodiments, the CAR may further comprise a signal peptide, such as the exemplary signal peptide provided by SEQ ID NO: 19.

In some embodiments, a CAR comprises an amino acid sequence shown in SEQ ID NO: 39, or an amino acid sequence that is at least 70%, at least 75%, at least 80%, at least 85%, at least 90%, at least 95%, or at least 99% identical to the amino acid sequence shown in SEQ ID NO: 39.

Amino acid sequence of IL3-D110A, K116V-nocostim
(SEQ ID NO: 39)
VNCSNMIDEIITHLKQPPLPLLDFNNLNGEDQDILMENNLRRPNLEAFN
RAVKSLQNASAIESILKNLLPCLPLATAAPTRHPIHIKAGDWNEFRRKL
TFYLVTLEESKYGPPCPPCPAPEFLGGPSVFLFPPKPKDTLMISRTPEV
TCVVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFL
YSRLTVDKSRWQEGNVFSCSVMHEALHNHYTQKSLSLSLGKMALIVLGG
VAGLLLFIGLGIFFRVKFSRSADAPAYQQGQNQLYNELNLGRREEYDVL
DKRRGRDPEMGGKPRRKNPQEGLYNELQKDKMAEAYSEIGMKGERRRGK
GHDGLYQGLSTATKDTYDALHMQALPPR

In some embodiments, a CAR is encoded by a nucleic acid sequence that comprises the sequence that is shown in SEQ ID NO: 40, or in a nucleic acid sequence that is at least 70%, at least 75%, at least 80%, at least 85%, at least 90%, at least 95%, or at least 99% identical to the nucleic acid sequence shown in SEQ ID NO: 40.

Nucleic acid sequence of IL3-D110A, K116V-nocostim
(SEQ ID NO: 40)
GTGAACTGCAGCAACATGATCGACGAGATCATCACCCACCTGAAGCAGCC
CCCCCTGCCCCTGCTGGACTTCAACAACCTGAACGGCGAGGACCAGGACA
TCCTGATGGAGAACAACCTGAGGAGGCCCAACCTGGAGGCCTTCAACAGG
GCCGTGAAGAGCCTGCAGAACGCCAGCGCCATCGAGAGCATCCTGAAGAA
CCTGCTGCCCTGCCTGCCCCTGGCCACCGCCGCCCCCACCAGGCACCCCA
TCCACATCAAGGCCGGCGACTGGAACGAGTTCAGGAGGAAGCTGACCTTC
TACCTGGTGACCCTGGAGGAGAGCAAGTACGGCCCCCCCTGCCCCCCCTG
CCCCGCCCCCGAGTTCCTGGGCGGCCCCAGCGTGTTCCTGTTCCCCCCCA
AGCCCAAGGACACCCTGATGATCAGCAGGACCCCCGAGGTGACCTGCGTG
GTGGTGGACGTGAGCCAGGAGGACCCCGAGGTGCAGTTCAACTGGTACGT
GGACGGCGTGGAGGTGCACAACGCCAAGACCAAGCCCAGGGAGGAGCAGT
TCAACAGCACCTACAGGGTGGTGAGCGTGCTGACCGTGCTGCACCAGGAC
TGGCTGAACGGCAAGGAGTACAAGTGCAAGGTGAGCAACAAGGGCCTGCC
CAGCAGCATCGAGAAGACCATCAGCAAGGCCAAGGGCCAGCCCAGGGAGC
CCCAGGTGTACACCCTGCCCCCCAGCCAGGAGGAGATGACCAAGAACCAG
GTGAGCCTGACCTGCCTGGTGAAGGGCTTCTACCCCAGCGACATCGCCGT
GGAGTGGGAGAGCAACGGCCAGCCCGAGAACAACTACAAGACCACCCCCC
CCGTGCTGGACAGCGACGGCAGCTTCTTCCTGTACAGCAGGCTGACCGTG
GACAAGAGCAGGTGGCAGGAGGGCAACGTGTTCAGCTGCAGCGTGATGCA
CGAGGCCCTGCACAACCACTACACCCAGAAGAGCCTGAGCCTGAGCCTGG
GCAAGATGGCCCTGATCGTGCTGGGCGGCGTGGCCGGCCTGCTGCTGTTC
ATCGGCCTGGGCATCTTCTTCAGGGTGAAGTTCAGCAGGAGCGCCGACGC
CCCCGCCTACCAGCAGGGCCAGAACCAGCTGTACAACGAGCTGAACCTGG
GCAGGAGGGAGGAGTACGACGTGCTGGACAAGAGGAGGGGCAGGGACCCC
GAGATGGGCGGCAAGCCCAGGAGGAAGAACCCCCAGGAGGGCCTGTACAA
CGAGCTGCAGAAGGACAAGATGGCCGAGGCCTACAGCGAGATCGGCATGA
AGGGCGAGAGGAGGAGGGGCAAGGGCCACGACGGCCTGTACCAGGGCCTG
AGCACCGCCACCAAGGACACCTACGACGCCCTGCACATGCAGGCCCTGCC
CCCCAGG

In some embodiments, a CAR construct as shown in SEQ ID NO: 40 is included in a vector. In some embodiments the recombinant expression vector includes a promoter (e.g., an SFFV promoter or an EF1α promoter).

### 11. IL3-D101A, K116V-41BBcostim

An exemplary CAR construct, as described herein, comprises a CD123-binding fragment containing D101A and K116V mutations, a IgG4 hinge domain, a CD4 transmembrane domain, a 4-1BB costimulatory signaling domain, and a CD3ζ intracellular signaling domain. In some embodiments, the CAR may further comprise a signal peptide, such as the exemplary signal peptide provided by SEQ ID NO: 19.

In some embodiments, a CAR comprises an amino acid sequence shown in SEQ ID NO: 41, or an amino acid sequence that is at least 70%, at least 75%, at least 80%, at least 85%, at least 90%, at least 95%, or at least 99% identical to the amino acid sequence shown in SEQ ID NO: 41.

Amino acid sequence of IL3-D101A, K116V-41BBcostim
(SEQ ID NO: 41)
VNCSNMIDEIITHLKQPPLPLLDFNNLNGEDQDILMENNLRRPNLEAFNR
AVKSLQNASAIESILKNLLPCLPLATAAPTRHPIHIKAGDWNEFRRKLTF
YLVTLEESKYGPPCPPCPAPEFLGGPSVFLFPPKPKDTLMISRTPEVTCV
VVDVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTV
DKSRWQEGNVFSCSVMHEALHNHYTQKSLSLSLGKMALIVLGGVAGLLLF
IGLGIFFKRGRKKLLYIFKQPFMRPVQTTQEEDGCSCRFPEEEEGGCELR
VKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTY
DALHMQALPPR

In some embodiments, a CAR is encoded by a nucleic acid sequence that comprises the sequence that is shown in SEQ ID NO: 42, or in a nucleic acid sequence that is at least 70%, at least 75%, at least 80%, at least 85%, at least 90%, at least 95%, or at least 99% identical to the nucleic acid sequence shown in SEQ ID NO: 42.

Nucleic acid sequence of IL3-D101A, K116V-
41BBcostim
(SEQ ID NO: 42)
GTGAACTGCAGCAACATGATCGACGAGATCATCACCCACCTGAAGCAGCC
CCCCCTGCCCCTGCTGGACTTCAACAACCTGAACGGCGAGGACCAGGACA
TCCTGATGGAGAACAACCTGAGGAGGCCCAACCTGGAGGCCTTCAACAGG
GCCGTGAAGAGCCTGCAGAACGCCAGCGCCATCGAGAGCATCCTGAAGAA
CCTGCTGCCCTGCCTGCCCCTGGCCACCGCCGCCCCCACCAGGCACCCCA
TCCACATCAAGGCCGGCGACTGGAACGAGTTCAGGAGGAAGCTGACCTTC
TACCTGGTGACCCTGGAGGAGAGCAAGTACGGCCCCCCCTGCCCCCCCTG
CCCCGCCCCCGAGTTCCTGGGCGGCCCCAGCGTGTTCCTGTTCCCCCCCA
AGCCCAAGGACACCCTGATGATCAGCAGGACCCCCGAGGTGACCTGCGTG
GTGGTGGACGTGAGCCAGGAGGACCCCGAGGTGCAGTTCAACTGGTACGT
GGACGGCGTGGAGGTGCACAACGCCAAGACCAAGCCCAGGGAGGAGCAGT
TCAACAGCACCTACAGGGTGGTGAGCGTGCTGACCGTGCTGCACCAGGAC
TGGCTGAACGGCAAGGAGTACAAGTGCAAGGTGAGCAACAAGGGCCTGCC
CAGCAGCATCGAGAAGACCATCAGCAAGGCCAAGGGCCAGCCCAGGGAGC
CCCAGGTGTACACCCTGCCCCCCAGCCAGGAGGAGATGACCAAGAACCAG
GTGAGCCTGACCTGCCTGGTGAAGGGCTTCTACCCCAGCGACATCGCCGT
GGAGTGGGAGAGCAACGGCCAGCCCGAGAACAACTACAAGACCACCCCCC
CCGTGCTGGACAGCGACGGCAGCTTCTTCCTGTACAGCAGGCTGACCGTG
GACAAGAGCAGGTGGCAGGAGGGCAACGTGTTCAGCTGCAGCGTGATGCA
CGAGGCCCTGCACAACCACTACACCCAGAAGAGCCTGAGCCTGAGCCTGG
GCAAGATGGCCCTGATCGTGCTGGGCGGCGTGGCCGGCCTGCTGCTGTTC
ATCGGCCTGGGCATCTTCTTCAAGAGGGGCAGGAAGAAGCTGCTGTACAT
CTTCAAGCAGCCCTTCATGAGGCCCGTGCAGACCACCCAGGAGGAGGACG
GCTGCAGCTGCAGGTTCCCCGAGGAGGAGGAGGGCGGCTGCGAGCTGAGG
GTGAAGTTCAGCAGGAGCGCCGACGCCCCCGCCTACCAGCAGGGCCAGAA
CCAGCTGTACAACGAGCTGAACCTGGGCAGGAGGGAGGAGTACGACGTGC
TGGACAAGAGGAGGGGCAGGGACCCCGAGATGGGCGGCAAGCCCAGGAGG
AAGAACCCCCAGGAGGGCCTGTACAACGAGCTGCAGAAGGACAAGATGGC
CGAGGCCTACAGCGAGATCGGCATGAAGGGCGAGAGGAGGAGGGGCAAGG
GCCACGACGGCCTGTACCAGGGCCTGAGCACCGCCACCAAGGACACCTAC
GACGCCCTGCACATGCAGGCCCTGCCCCCCAGG

In some embodiments, a CAR construct as shown in SEQ ID NO: 42 is included in a vector. In some embodiments the recombinant expression vector includes a promoter (e.g., an SFFV promoter or an EF1α promoter).

### 12. IL3-D101A, K116V-CD28 Costim

An exemplary CAR construct, as described herein, comprises a CD123-binding fragment containing D101A and K116V mutations, a IgG4 hinge domain, a CD4 transmembrane domain, a CD28 costimulatory signaling domain, and a CD3ζ intracellular signaling domain. In some embodiments, the CAR may further comprise a signal peptide, such as the exemplary signal peptide provided by SEQ ID NO: 19.

In some embodiments, a CAR comprises an amino acid sequence shown in SEQ ID NO: 43, or an amino acid sequence that is at least 70%, at least 75%, at least 80%, at least 85%, at least 90%, at least 95%, or at least 99% identical to the amino acid sequence shown in SEQ ID NO: 43.

Amino acid sequence of IL3-D101A, K116V-CD28costim
(SEQ ID NO: 43)
VNCSNMIDEIITHLKQPPLPLLDFNNLNGEDQDILMENNLRRPNLEAFNR
AVKSLQNASAIESILKNLLPCLPLATAAPTRHPIHIKAGDWNEFRRKLTF
YLVTLEESKYGPPCPPCPAPEFLGGPSVFLFPPKPKDTLMISRTPEVTCV
VVDVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTV
DKSRWQEGNVFSCSVMHEALHNHYTQKSLSLSLGKMALIVLGGVAGLLLF
IGLGIFFRSKRSRLLHSDYMNMTPRRPGPTRKHQYPYAPPRDFAAYRSRV
KFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYD
ALHMQALPPR

In some embodiments, a CAR is encoded by a nucleic acid sequence that comprises the sequence that is shown in SEQ ID NO: 44, or in a nucleic acid sequence that is at least 70%, at least 75%, at least 80%, at least 85%, at least 90%, at least 95%, or at least 99% identical to the nucleic acid sequence shown in SEQ ID NO: 44.

Nucleic acid sequence of IL3-D101A, K116V-
CD28costim
(SEQ ID NO: 44)
GTGAACTGCAGCAACATGATCGACGAGATCATCACCCACCTGAAGCAGCC
CCCCCTGCCCCTGCTGGACTTCAACAACCTGAACGGCGAGGACCAGGACA
TCCTGATGGAGAACAACCTGAGGAGGCCCAACCTGGAGGCCTTCAACAGG
GCCGTGAAGAGCCTGCAGAACGCCAGCGCCATCGAGAGCATCCTGAAGAA
CCTGCTGCCCTGCCTGCCCCTGGCCACCGCCGCCCCCACCAGGCACCCCA
TCCACATCAAGGCCGGCGACTGGAACGAGTTCAGGAGGAAGCTGACCTTC
TACCTGGTGACCCTGGAGGAGAGCAAGTACGGCCCCCCCTGCCCCCCCTG
CCCCGCCCCCGAGTTCCTGGGCGGCCCCAGCGTGTTCCTGTTCCCCCCCA
AGCCCAAGGACACCCTGATGATCAGCAGGACCCCCGAGGTGACCTGCGTG
GTGGTGGACGTGAGCCAGGAGGACCCCGAGGTGCAGTTCAACTGGTACGT
GGACGGCGTGGAGGTGCACAACGCCAAGACCAAGCCCAGGGAGGAGCAGT
TCAACAGCACCTACAGGGTGGTGAGCGTGCTGACCGTGCTGCACCAGGAC
TGGCTGAACGGCAAGGAGTACAAGTGCAAGGTGAGCAACAAGGGCCTGCC
CAGCAGCATCGAGAAGACCATCAGCAAGGCCAAGGGCCAGCCCAGGGAGC
CCCAGGTGTACACCCTGCCCCCCAGCCAGGAGGAGATGACCAAGAACCAG
GTGAGCCTGACCTGCCTGGTGAAGGGCTTCTACCCCAGCGACATCGCCGT
GGAGTGGGAGAGCAACGGCCAGCCCGAGAACAACTACAAGACCACCCCCC
CCGTGCTGGACAGCGACGGCAGCTTCTTCCTGTACAGCAGGCTGACCGTG
GACAAGAGCAGGTGGCAGGAGGGCAACGTGTTCAGCTGCAGCGTGATGCA
CGAGGCCCTGCACAACCACTACACCCAGAAGAGCCTGAGCCTGAGCCTGG
GCAAGATGGCCCTGATCGTGCTGGGCGGCGTGGCCGGCCTGCTGCTGTTC
ATCGGCCTGGGCATCTTCTTCAGGAGCAAGAGGAGCAGGCTGCTGCACAG
CGACTACATGAACATGACCCCCAGGAGGCCCGGCCCCACCAGGAAGCACC
AGTACCCCTACGCCCCCCCCAGGGACTTCGCCGCCTACAGGAGCAGGGTG
AAGTTCAGCAGGAGCGCCGACGCCCCCGCCTACCAGCAGGGCCAGAACCA
GCTGTACAACGAGCTGAACCTGGGCAGGAGGGAGGAGTACGACGTGCTGG
ACAAGAGGAGGGGCAGGGACCCCGAGATGGGCGGCAAGCCCAGGAGGAAG
AACCCCCAGGAGGGCCTGTACAACGAGCTGCAGAAGGACAAGATGGCCGA
GGCCTACAGCGAGATCGGCATGAAGGGCGAGAGGAGGAGGGGCAAGGGCC
ACGACGGCCTGTACCAGGGCCTGAGCACCGCCACCAAGGACACCTACGAC
GCCCTGCACATGCAGGCCCTGCCCCCCAGG

In some embodiments, a CAR construct as shown in SEQ ID NO: 44 is included in a vector. In some embodiments the recombinant expression vector includes a promoter (e.g., an SFFV promoter or an EF1α promoter).

### 13. IL3-D101A, K116V-Nocostim+CD8 Transmembrane

An exemplary CAR construct, as described herein, comprises a CD123-binding fragment containing D101A and K116V mutations, a IgG4 hinge domain, a CD8 transmembrane domain, and a CD3ζ intracellular signaling domain. In some embodiments, the CAR may further comprise a signal peptide, such as the exemplary signal peptide provided by SEQ ID NO: 19.

In some embodiments, a CAR comprises an amino acid sequence shown in SEQ ID NO: 45, or an amino acid sequence that is at least 70%, at least 75%, at least 80%, at least 85%, at least 90%, at least 95%, or at least 99% identical to the amino acid sequence shown in SEQ ID NO: 45.

Amino acid sequence of IL3-D101A, K116V-
nocostim + CD8 transmembrane
(SEQ ID NO: 45)
VNCSNMIDEIITHLKQPPLPLLDFNNLNGEDQDILMENNLRRPNLEAFNR
AVKSLQNASAIESILKNLLPCLPLATAAPTRHPIHIKAGDWNEFRRKLTF
YLVTLEESKYGPPCPPCPAPEFLGGPSVFLFPPKPKDTLMISRTPEVTCV
VVDVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTV
DKSRWQEGNVFSCSVMHEALHNHYTQKSLSLSLGKIYIWAPLAGTCGVLL
LSLVITLYCRVKFSRSADAPAYQQGQNQLYNELNLGRREEYDVLDKRRGR
DPEMGGKPRRKNPQEGLYNELQKDKMAEAYSEIGMKGERRRGKGHDGLYQ
GLSTATKDTYDALHMQALPPR

In some embodiments, a CAR in encoded by a nucleic acid sequence that comprises the sequence that is shown in SEQ ID NO: 46, or in a nucleic acid sequence that is at least 70%, at least 75%, at least 80%, at least 85%, at least 90%, at least 95%, or at least 99% identical to the nucleic acid sequence shown in SEQ ID NO: 46.

Nucleic acid sequence of IL3-D101A, K116V-
nocostim + CD8 transmembrane
(SEQ ID NO: 46)
GTGAACTGCAGCAACATGATCGACGAGATCATCACCCACCTGAAGCAGCC
CCCCCTGCCCCTGCTGGACTTCAACAACCTGAACGGCGAGGACCAGGACA
TCCTGATGGAGAACAACCTGAGGAGGCCCAACCTGGAGGCCTTCAACAGG
GCCGTGAAGAGCCTGCAGAACGCCAGCGCCATCGAGAGCATCCTGAAGAA
CCTGCTGCCCTGCCTGCCCCTGGCCACCGCCGCCCCCACCAGGCACCCCA
TCCACATCAAGGCCGGCGACTGGAACGAGTTCAGGAGGAAGCTGACCTTC
TACCTGGTGACCCTGGAGGAGAGCAAGTACGGCCCCCCCTGCCCCCCCTG
CCCCGCCCCCGAGTTCCTGGGCGGCCCCAGCGTGTTCCTGTTCCCCCCCA
AGCCCAAGGACACCCTGATGATCAGCAGGACCCCCGAGGTGACCTGCGTG
GTGGTGGACGTGAGCCAGGAGGACCCCGAGGTGCAGTTCAACTGGTACGT
GGACGGCGTGGAGGTGCACAACGCCAAGACCAAGCCCAGGGAGGAGCAGT
TCAACAGCACCTACAGGGTGGTGAGCGTGCTGACCGTGCTGCACCAGGAC
TGGCTGAACGGCAAGGAGTACAAGTGCAAGGTGAGCAACAAGGGCCTGCC
CAGCAGCATCGAGAAGACCATCAGCAAGGCCAAGGGCCAGCCCAGGGAGC
CCCAGGTGTACACCCTGCCCCCCAGCCAGGAGGAGATGACCAAGAACCAG
GTGAGCCTGACCTGCCTGGTGAAGGGCTTCTACCCCAGCGACATCGCCGT
GGAGTGGGAGAGCAACGGCCAGCCCGAGAACAACTACAAGACCACCCCCC
CCGTGCTGGACAGCGACGGCAGCTTCTTCCTGTACAGCAGGCTGACCGTG
GACAAGAGCAGGTGGCAGGAGGGCAACGTGTTCAGCTGCAGCGTGATGCA
CGAGGCCCTGCACAACCACTACACCCAGAAGAGCCTGAGCCTGAGCCTGG
GCAAGATCTACATCTGGGCCCCCCTGGCCGGCACCTGCGGCGTGCTGCTG
CTGAGCCTGGTGATCACCCTGTACTGCAGGGTGAAGTTCAGCAGGAGCGC
CGACGCCCCCGCCTACCAGCAGGGCCAGAACCAGCTGTACAACGAGCTGA
ACCTGGGCAGGAGGGAGGAGTACGACGTGCTGGACAAGAGGAGGGGCAGG
GACCCCGAGATGGGCGGCAAGCCCAGGAGGAAGAACCCCCAGGAGGGCCT
GTACAACGAGCTGCAGAAGGACAAGATGGCCGAGGCCTACAGCGAGATCG
GCATGAAGGGCGAGAGGAGGAGGGGCAAGGGCCACGACGGCCTGTACCAG
GGCCTGAGCACCGCCACCAAGGACACCTACGACGCCCTGCACATGCAGGC
CCTGCCCCCCAGG

In some embodiments, a CAR construct as shown in SEQ ID NO: 46 is included in a vector. In some embodiments the recombinant expression vector includes a promoter (e.g., an SFFV promoter or an EF1α promoter).

### 14. IL3-D101A, K116V-Nocostim+IgG4 L235E, N297Q

An exemplary CAR construct, as described herein, comprises a CD123-binding fragment containing D101A and K116V mutations, a IgG4 hinge domain containing L235E and N297 mutations, a CD4 transmembrane domain, and a CD3ζ intracellular signaling domain. In some embodiments, the CAR may further comprise a signal peptide, such as the exemplary signal peptide provided by SEQ ID NO: 19.

In some embodiments, a CAR comprises an amino acid sequence shown in SEQ ID NO: 47, or an amino acid sequence that is at least 70%, at least 75%, at least 80%, at least 85%, at least 90%, at least 95%, or at least 99% identical to the amino acid sequence shown in SEQ ID NO: 47.

Amino acid sequence of IL3-D101A, K116V-nocostim +
IgG4 L235E, N297Q
(SEQ ID NO: 47)
VNCSNMIDEIITHLKQPPLPLLDFNNLNGEDQDILMENNLRRPNLEAFNR
AVKSLQNASAIESILKNLLPCLPLATAAPTRHPIHIKAGDWNEFRRKLTF
YLVTLEESKYGPPCPSCPAPEFEGGPSVFLPPKPKDTLMISRTPEVTCVV
VDVSQEDPEVQFNWYVDGVEVHQAKTKPREEQFNSTYRVVSVLTVLHQDW
LNGKEYKCKVSNKGLPSSIEKTISKAKGQPREPQVYTLPPSQEEMTKNQV
SLTCLVKGFYPSDIAVEWESNGQPENNYKTTPPVLDSDGSFFLYSRLTVD
KSRWQEGNVFSCSVMHEALHNHYTQKSLSLSLGKMALIVLGGVAGLLLFI
GLGIFFRVKFSRSADAPAYQQGQNQLYNELNLGRREEYDVLDKRRGRDPE
MGGKPRRKNPQEGLYNELQKDKMAEAYSEIGMKGERRRGKGHDGLYQGLS
TATKDTYDALHMQALPPR

In some embodiments, a CAR in encoded by a nucleic acid sequence that comprises the sequence that is shown in SEQ ID NO: 48, or in a nucleic acid sequence that is at least 70%, at least 75%, at least 80%, at least 85%, at least 90%, at least 95%, or at least 99% identical to the nucleic acid sequence shown in SEQ ID NO: 48.

Nucleic acid sequence of IL3-D101A, K116V-
nocostim + IgG4 L235E, N297
(SEQ ID NO: 48)
GTGAACTGCAGCAACATGATCGACGAGATCATCACCCACCTGAAGCAGCC
TCCTCTGCCTCTGCTGGACTTCAACAACCTGAACGGCGAGGACCAGGACA
TCCTGATGGAGAACAACCTGAGGAGGCCTAACCTGGAGGCCTTCAACAGG
GCCGTGAAGAGCCTGCAGAACGCCAGCGCCATCGAGAGCATCCTGAAGAA
CCTGCTGCCTTGCCTGCCTCTGGCCACCGCCGCCCCTACCAGGCACCCTA
TCCACATCAAGGCCGGCGACTGGAACGAGTTCAGGAGGAAGCTGACCTTC
TACCTGGTGACCCTGGAGGAGAGCAAGTACGGCCCTCCTTGCCCTAGCTG
CCCTGCCCCTGAGTTCGAGGGCGGCCCTAGCGTGTTCCTGCCTCCTAAGC
CTAAGGACACCCTGATGATCAGCAGGACCCCTGAGGTGACCTGCGTGGTG
GTGGACGTGAGCCAGGAGGACCCTGAGGTGNNNTTCAACTGGTACGTGGA
CGGCGTGGAGGTGCACNNNGCCAAGACCAAGCCTAGGGAGGAGCAGTTCA
ACAGCACCTACAGGGTGGTGAGCGTGCTGACCGTGCTGCACNNNGACTGG
CTGAACGGCAAGGAGTACAAGTGCAAGGTGAGCAACAAGGGCCTGCCTAG
CAGCATCGAGAAGACCATCAGCAAGGCCAAGGGCCAGCCTAGGGAGCCTN
NNGTGTACACCCTGCCTCCTAGCNNNGAGGAGATGACCAAGAACNNNGTG
AGCCTGACCTGCCTGGTGAAGGGCTTCTACCCTAGCGACATCGCCGTGGA
GTGGGAGAGCAACGGCNNNCCTGAGAACAACTACAAGACCACCCCTCCTG
TGCTGGACAGCGACGGCAGCTTCTTCCTGTACAGCAGGCTGACCGTGGAC
AAGAGCAGGTGGCAGGAGGGCAACGTGTTCAGCTGCAGCGTGATGCACGA
GGCCCTGCACAACCACTACACCNNNAAGAGCCTGAGCCTGAGCCTGGGCA
AGATGGCCCTGATCGTGCTGGGCGGCGTGGCCGGCCTGCTGCTGTTCATC
GGCCTGGGCATCTTCTTCAGGGTGAAGTTCAGCAGGAGCGCCGACGCCCC
TGCCTACCAGCAGGGCCAGAACCAGCTGTACAACGAGCTGAACCTGGGCA
GGAGGGAGGAGTACGACGTGCTGGACAAGAGGAGGGGCAGGGACCCTGAG
ATGGGCGGCAAGCCTAGGAGGAAGAACCCTCAGGAGGGCCTGTACAACGA
GCTGCAGAAGGACAAGATGGCCGAGGCCTACAGCGAGATCGGCATGAAGG
GCGAGAGGAGGAGGGGCAAGGGCCACGACGGCCTGTACCAGGGCCTGAGC
ACCGCCACCAAGGACACCTACGACGCCCTGCACATGCAGGCCCTGCCTCC
TAGG

In some embodiments, a CAR construct as shown in SEQ ID NO: 48 is included in a vector. In some embodiments the recombinant expression vector includes a promoter (e.g., an SFFV promoter or an EF1α promoter).

### 15. IL3-D101A, K116V-Nocostim+CD8 Hinge

An exemplary CAR construct, as described herein, comprises a CD123-binding fragment containing D101A and K116V mutations, a IgG4 hinge domain, a CD4 transmembrane domain, and a CD3ζ intracellular signaling domain. In some embodiments, the CAR may further comprise a signal peptide, such as the exemplary signal peptide provided by SEQ ID NO: 19.

In some embodiments, a CAR comprises an amino acid sequence shown in SEQ ID NO: 49, or an amino acid sequence that is at least 70%, at least 75%, at least 80%, at least 85%, at least 90%, at least 95%, or at least 99% identical to the amino acid sequence shown in SEQ ID NO: 49.

Amino acid sequence of IL3-D101A, K116V-nocostim +
CD8 hinge
(SEQ ID NO: 49)
VNCSNMIDEIITHLKQPPLPLLDFNNLNGEDQDILMENNLRRPNLEAFNR
AVKSLQNASAIESILKNLLPCLPLATAAPTRHPIHIKAGDWNEFRRKLTF
YLVTLETTTPAPRPPTPAPTIASQPLSLRPEACRPAAGGAVHTRGLDFAC
DMALIVLGGVAGLLLFIGLGIFFRVKFSRSADAPAYQQGQNQLYNELNLG
RREEYDVLDKRRGRDPEMGGKPRRKNPQEGLYNELQKDKMAEAYSEIGMK
GERRRGKGHDGLYQGLSTATKDTYDALHMQALPPR

In some embodiments, a CAR in encoded by a nucleic acid sequence that comprises the sequence that is shown in SEQ ID NO: 50, or in a nucleic acid sequence that is at least 70%, at least 75%, at least 80%, at least 85%, at least 90%, at least 95%, or at least 99% identical to the nucleic acid sequence shown in SEQ ID NO: 50.

Nucleic acid sequence of IL3-D101A, K116V-
nocostim + CD8 hinge
(SEQ ID NO: 50)
ATGCTGCTGCTGGTGACCAGCCTGCTGCTGTGCGAGCTGCCCCACCCCGC
CTTCCTGCTGATCGTGAACTGCAGCAACATGATCGACGAGATCATCACCC
ACCTGAAGCAGCCCCCCCTGCCCCTGCTGGACTTCAACAACCTGAACGGC
GAGGACCAGGACATCCTGATGGAGAACAACCTGAGGAGGCCCAACCTGGA
GGCCTTCAACAGGGCCGTGAAGAGCCTGCAGAACGCCAGCGCCATCGAGA
GCATCCTGAAGAACCTGCTGCCCTGCCTGCCCCTGGCCACCGCCGCCCCC
ACCAGGCACCCCATCCACATCAAGGCCGGCGACTGGAACGAGTTCAGGAG
GAAGCTGACCTTCTACCTGGTGACCCTGGAGACCACCACCCCCGCCCCCA
GGCCCCCCACCCCCGCCCCCACCATCGCCAGCNNNCCCCTGAGCCTGAGG
CCCGAGGCCTGCAGGCCCGCCGCCGGCGGCGCCGTGCACACCAGGGGCCT
GGACTTCGCCTGCGACATGGCCCTGATCGTGCTGGGCGGCGTGGCCGGCC
TGCTGCTGTTCATCGGCCTGGGCATCTTCTTCAGGGTGAAGTTCAGCAGG
AGCGCCGACGCCCCCGCCTACCAGCAGGGCCAGAACCAGCTGTACAACGA
GCTGAACCTGGGCAGGAGGGAGGAGTACGACGTGCTGGACAAGAGGAGGG
GCAGGGACCCCGAGATGGGCGGCAAGCCCAGGAGGAAGAACCCCCAGGAG
GGCCTGTACAACGAGCTGCAGAAGGACAAGATGGCCGAGGCCTACAGCGA
GATCGGCATGAAGGGCGAGAGGAGGAGGGGCAAGGGCCACGACGGCCTGT
ACCAGGGCCTGAGCACCGCCACCAAGGACACCTACGACGCCCTGCACATG
CAGGCCCTGCCCCCCAGG

In some embodiments, a CAR construct as shown in SEQ ID NO: 50 is included in a vector. In some embodiments the recombinant expression vector includes a promoter (e.g., an SFFV promoter or an EF1α promoter).

### 16. IL3-K116W-Nocostim

An exemplary CAR construct, as described herein, comprises a CD123-binding fragment containing a K116W mutation, a linker region, a IgG4 hinge domain, a CD4 transmembrane domain, and a CD3ζ intracellular signaling domain. In some embodiments, the CAR may further comprise a signal peptide, such as the exemplary signal peptide provided by SEQ ID NO: 19.

In some embodiments, a CAR comprises an amino acid sequence shown in SEQ ID NO: 51, or an amino acid sequence that is at least 70%, at least 75%, at least 80%, at least 85%, at least 90%, at least 95%, or at least 99% identical to the amino acid sequence shown in SEQ ID NO: 51.

Amino acid sequence of IL3- K116W-nocostim
(SEQ ID NO: 51)
VNCSNMIDEIITHLKQPPLPLLDFNNLNGEDQDILMENNLRRPNLEAFNR
AVKSLQNASAIESILKNLLPCLPLATAAPTRHPIHIKDGDWNEFRRKLTF
YLWTLEGGGGSGGGGSESKYGPPCPPCPAPEFLGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGKMALIV
LGGVAGLLLFIGLGIFFRVKFSRSADAPAYQQGQNQLYNELNLGRREEYD
VLDKRRGRDPEMGGKPRRKNPQEGLYNELQKDKMAEAYSEIGMKGERRRG
KGHDGLYQGLSTATKDTYDALHMQALPPR

In some embodiments, a CAR in encoded by a nucleic acid sequence that comprises the sequence that is shown in SEQ ID NO: 52, or in a nucleic acid sequence that is at least 70%, at least 75%, at least 80%, at least 85%, at least 90%, at least 95%, or at least 99% identical to the nucleic acid sequence shown in SEQ ID NO: 52.

Nucleic acid sequence of IL3- K116W-nocostim
(SEQ ID NO: 52)
GTGAACTGCAGCAACATGATCGACGAGATCATCACCCACCTGAAGCAGCC
CCCCCTGCCCCTGCTGGACTTCAACAACCTGAACGGCGAGGACCAGGACA
TCCTGATGGAGAACAACCTGAGAAGACCCAACCTGGAGGCCTTCAACAGA
GCCGTGAAGAGCCTGCAGAACGCCAGCGCCATCGAGAGCATCCTGAAGAA
CCTGCTGCCCTGCCTGCCCCTGGCCACCGCCGCCCCCACCAGACACCCCA
TCCACATCAAGGACGGCGACTGGAACGAGTTCAGAAGAAAGCTGACCTTC
TACCTGTGGACCCTGGAGGGCGGCGGCGGCAGCGGCGGCGGCGGCAGCGA
GAGCAAGTACGGCCCCCCCTGCCCCCCCTGCCCCGCCCCCGAGTTCCTGG
GCGGCCCCAGCGTGTTCCTGTTCCCCCCCAAGCCCAAGGACACCCTGATG
ATCAGCAGAACCCCCGAGGTGACCTGCGTGGTGGTGGACGTGAGCCAGGA
GGACCCCGAGGTGCAGTTCAACTGGTACGTGGACGGCGTGGAGGTGCACA
ACGCCAAGACCAAGCCCAGAGAGGAGCAGTTCAACAGCACCTACAGAGTG
GTGAGCGTGCTGACCGTGCTGCACCAGGACTGGCTGAACGGCAAGGAGTA
CAAGTGCAAGGTGAGCAACAAGGGCCTGCCCAGCAGCATCGAGAAGACCA
TCAGCAAGGCCAAGGGCCAGCCCAGAGAGCCCCAGGTGTACACCCTGCCC
CCCAGCCAGGAGGAGATGACCAAGAACCAGGTGAGCCTGACCTGCCTGGT
GAAGGGCTTCTACCCCAGCGACATCGCCGTGGAGTGGGAGAGCAACGGCC
AGCCCGAGAACAACTACAAGACCACCCCCCCCGTGCTGGACAGCGACGGC
AGCTTCTTCCTGTACAGCAGACTGACCGTGGACAAGAGCAGATGGCAGGA
GGGCAACGTGTTCAGCTGCAGCGTGATGCACGAGGCCCTGCACAACCACT
ACACCCAGAAGAGCCTGAGCCTGAGCCTGGGCAAGATGGCCCTGATCGTG
CTGGGCGGCGTGGCCGGCCTGCTGCTGTTCATCGGCCTGGGCATCTTCTT
CAGAGTGAAGTTCAGCAGAAGCGCCGACGCCCCCGCCTACCAGCAGGGCC
AGAACCAGCTGTACAACGAGCTGAACCTGGGCAGAAGAGAGGAGTACGAC
GTGCTGGACAAGAGAAGAGGCAGAGACCCCGAGATGGGCGGCAAGCCCAG
AAGAAAGAACCCCCAGGAGGGCCTGTACAACGAGCTGCAGAAGGACAAGA
TGGCCGAGGCCTACAGCGAGATCGGCATGAAGGGCGAGAGAAGAAGAGGC
AAGGGCCACGACGGCCTGTACCAGGGCCTGAGCACCGCCACCAAGGACAC
CTACGACGCCCTGCACATGCAGGCCCTGCCCCCCAGA

In some embodiments, a CAR construct as shown in SEQ ID NO: 52 is included in a vector. In some embodiments the recombinant expression vector includes a promoter (e.g., an SFFV promoter or an EF1α promoter).

In some embodiments, any nucleotide sequences herein may be codon-optimized. Without being bound to a particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by RNA that is more readily available within a cell, thus increasing translation efficiency. Optimization of the nucleotide sequence may also reduce secondary mRNA structures that would interfere with translation, thus increasing translation efficiency. In an embodiment of the invention, the codon-optimized nucleotide sequence may comprise, consist, or consist essentially of any one of the nucleic acid sequences described herein.

Any of the nucleic acids of described herein may be recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

A recombinant nucleic acid may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Green et al., supra. The nucleic acids can be constructed based on chemical synthesis and/or enzymatic ligation reactions using procedures known in the art. See, for example, Green et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methyl guanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, CO) and Synthegen (Houston, TX).

The nucleic acids can comprise any isolated or purified nucleotide sequence which encodes any of the CARs or functional portions or functional variants thereof. Alternatively, the nucleotide sequence can comprise a nucleotide sequence which is degenerate to any of the sequences or a combination of degenerate sequences.

Also provided herein are isolated or purified nucleic acids comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. The term “high stringency conditions” refers to a nucleotide sequence that specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the CARs described herein. It is generally appreciated that conditions can be rendered more stringent by the addition of increasing amounts of formamide.

The present disclosure also provides nucleic acids comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein. Also within the scope of the present disclosure are functional portions of any of the CARs described herein.

### Therapeutic Methods

Aspects of the present disclosure provide methods of treating a disease, disorder, or condition associated in a subject comprising administering to the subject a therapeutically effective amount of any of the CARs, nucleic acids, cell expressing any of the CARs, or pharmaceutical compositions described herein. In some embodiments, the methods involve administering a therapeutically effective amount of a pharmaceutical composition comprising cells (e.g., a population of cells) expressing any of the CARs described herein. In some embodiments, the method is for treating cancer in a subject, comprising administering to the subject a therapeutically effective amount of a pharmaceutical composition comprising cells (e.g., a population of cells) expressing any of the CARs described herein. In some embodiments, the method is for treating a hematopoietic malignancy or pre-malignancy in a subject, comprising administering to the subject a therapeutically effective amount of a pharmaceutical composition comprising cells (e.g., a population of cells) expressing any of the CARs described herein.

In another aspect, the method is for stimulating an immune response to a target cell or tissue (e.g., a cancer, tumor cell) in a subject comprising administering to a subject a therapeutically effective amount of a pharmaceutical composition comprising cells (e.g., a population of cells) expressing any of the CARs described herein. In some embodiments, the method involves use of the modified cell described herein in the manufacture of a medicament for stimulating an immune response in a subject in need thereof. In some embodiments, the method involves use of any of the CARs, nucleic acids, cells expressing any of the CARs, or pharmaceutical compositions described herein in the manufacture of a medicament for the treatment of a cancer in a subject in need thereof. In some embodiments, the method involves use of any of the CARs, nucleic acids, cells expressing any of the CARs. or pharmaceutical compositions described herein in the manufacture of a medicament for the treatment of a hematopoietic malignancy or pre-malignancy in a subject in need thereof.

The modified cells (e.g., immune cells, such as T-lymphocytes, NK cells) generated as described herein possess targeted effector activity. In some embodiments, the modified cells have targeted effector activity directed against an antigen on a target cell, such as through specific binding of the IL-3 molecule or CD123-binding fragment thereof of any of the CARs described herein. In another embodiment, the targeted effector activity includes, but is not limited to, phagocytosis, targeted cellular cytotoxicity, antigen presentation, and cytokine secretion.

The CARs described herein (including functional portions and variants thereof), nucleic acids, vectors, and cells expressing any of the CARs described herein, e.g., immune cells, such as T-lymphocytes, NK cells (including populations thereof), are collectively referred to as “CAR materials.”

### Pharmaceutical Compositions

The CAR materials described herein can be formulated into a composition, such as a pharmaceutical composition. In some embodiments, the present disclosure provides a pharmaceutical composition comprising any of the CAR materials described herein and a pharmaceutically acceptable carrier. The pharmaceutical compositions containing any of the CAR materials can comprise more than one CAR material, e.g., a CAR, a nucleic acid, or two or more different CARs, cells expressing any of the CARs. Alternatively, the pharmaceutical composition can comprise CAR material in combination with other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the pharmaceutical composition comprises a cell expressing any of the CAR described herein, or populations of such cells.

With respect to pharmaceutical compositions, the pharmaceutically acceptable carrier can be any of those conventionally used and is limited only by chemico-physical considerations, such as solubility and lack of reactivity with the active agent(s), and by the route of administration. Pharmaceutically acceptable carriers described herein, for example, vehicles, adjuvants, excipients, and diluents, are well known to those skilled in the art and are readily available to the public. It is preferred that the pharmaceutically acceptable carrier be one which has no detrimental side effects or toxicity under the conditions of use.

The choice of carrier will be determined in part by the particular CAR material, as well as by the particular methods used to administer the CAR material, for example to a subject. Accordingly, there are a variety of suitable formulations of the pharmaceutical composition of the invention. Methods for preparing administrable (e.g., parenterally administrable) compositions are known or apparent to those skilled in the art and are described in more detail in, for example, Remington: The Science and Practice of Pharmacy, Pharmaceutical Press; 22nd ed. (2012).

The CAR materials, including pharmaceutical compositions comprising any of the CAR materials, may be administered in any suitable manner. In some embodiments, CAR materials are administered by injection, (e.g., subcutaneously, intravenously, intratumorally, intraarterially, intramuscularly, intradermally, interperitoneally, or intrathecally). In some embodiments, CAR materials, including pharmaceutical compositions comprising any of the CAR materials, are administered intravenously. In some embodiments, CAR materials, including pharmaceutical compositions comprising any of the CAR materials, are administered by infusion. A suitable pharmaceutically acceptable carrier for the CAR materials described herein for injection may include any isotonic carrier such as, for example, normal saline (about 0.90% w/v of NaCl in water, about 300 mOsm/L NaCl in water, or about 9.0 g NaCl per liter of water), NORMOSOL-R electrolyte solution (Abbott, Chicago, IL), PLASMA-LYTE A (Baxter, Deerfield, IL), about 5% dextrose in water, or Ringer's lactate. In some embodiments, the pharmaceutically acceptable carrier is supplemented with human serum albumen.

Amounts effective for a therapeutic or prophylactic use will depend on, for example, the stage and severity of the disease or disorder being treated, the age, weight, and general state of health of the patient, and the judgment of the prescribing physician. The size of the dose will also be determined by active selection, method of administration, timing and frequency of administration, the existence, nature, and extent of any adverse side-effects that might accompany the administration of a particular active, and the desired physiological effect. It will be appreciated by one of skill in the art that various diseases or disorders could require prolonged treatment involving multiple administrations, for example using the CAR materials described herein in each or various rounds of administration. By way of example and not intending to limit the invention, when the CAR material is a cell expressing any of the CARs described herein, an exemplary dose of the cells may be a minimum of one million cells (1×10⁶ cells/dose).

The amount or dose of the CAR material administered should be sufficient to effect a therapeutic or prophylactic response in the subject or animal over a reasonable time frame. For example, the dose of the CAR material should be sufficient to bind to antigen (i.e., CD123), or detect, treat, or prevent cancer or hematopoietic malignancy or pre-malignancy, including reducing one or more symptoms and/or delaying the progression of the disease, in a period of from about 2 hours or longer, e.g., about 12 to about 24 or more hours, such as for about 1 day to 6 months or longer, from the time of administration. In some embodiments, the time period could be even longer. The dose will be determined by factors such as the efficacy of the particular CAR material, the condition of the animal (e.g., human), including the body weight of the animal (e.g., human) to be treated, and the severity of the disease in the subject.

An assay, which comprises, for example, comparing the extent to which target cells are lysed and/or IFN-gamma or IL-2 is secreted by cells expressing any of the CARs described herein upon administration of a given dose of such cells to a subject, among a set of subjects of which is each given a different dose of the immune cells, could be used to determine a starting dose to be administered to a subject. The extent to which target cells are lysed and/or IFN-gamma or IL-2 is secreted upon administration of a certain dose can be assayed by methods known in the art.

When the CAR materials are administered with one or more additional therapeutic agents, one or more additional therapeutic agents can be coadministered to a subject. The term “coadministering” refers to administering one or more additional therapeutic agents and the CAR materials sufficiently close in time such that the CAR materials can enhance the effect of one or more additional therapeutic agents, or vice versa. In this regard, CAR construct materials can be administered first and the one or more additional therapeutic agents can be administered second, or vice versa. Alternatively, CAR construct materials and the one or more additional therapeutic agents can be administered simultaneously. An exemplary therapeutic agent that may be coadministered with the CAR materials is IL-2.

It is contemplated that the CAR construct materials described herein can be used in methods of treating or preventing a disease in a subject. Without being bound to a particular theory or mechanism, the CAR constructs have biological activity, e.g., CARs that recognize antigen, i.e., CD123, such that the CARs, when expressed by a cell, are able to mediate an immune response against the cell expressing the antigen, i.e., CD123. In this regard, in some embodiments, the methods of treating or preventing a disease, disorder, or condition in a subject (e.g., cancer, hematopoietic malignancy or pre-malignancy) comprising administering to the subject any of the CARs, nucleic acids, vectors, cells expressing the CARs or populations of such cells, and/or any of the pharmaceutical compositions described herein in an amount effective to treat or prevent the disease, disorder, or condition in a subject (e.g., cancer, hematopoietic malignancy or pre-malignancy) in the subject.

In some embodiments, the method further comprises lymphodepleting the subject prior to administering any of the CAR materials described herein. Examples of lymphodepletion include, but may not be limited to, nonmyeloablative lymphodepleting chemotherapy, myeloablative lymphodepleting chemotherapy, total body irradiation, etc.

In some embodiments, the cells expressing the CARs, or populations of such cells, allogeneic or autologous to the subject.

With respect to the methods of treatment, in some embodiments, the disease, disorder, or condition is cancer. The cancer can be any cancer, including any of acute lymphocytic cancer, acute myeloid leukemia (AML), alveola rhabdomyosarcoma, bladder cancer (e.g., bladder carcinoma), bone cancer, brain cancer (e.g., medulloblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle car, cancer of the oral cavity, cancer of the vulva, chronic lymphocytic leukemia (CLL), chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin lymphoma, hypopharynx cancer, kidney cancer, larynx cancer, leukemia, liquid tumors, liver cancer, lung cancer (e.g., non-small cell lung carcinoma), lymphoma, malignant mesothelioma, mastocytoma, melanoma, multiple myeloma, nasopharynx cancer, non-Hodgkin lymphoma, B-chronic lymphocytic leukemia, B-precursor acute lymphoblastic leukemia (B-ALL), pre-B cell precursor acute lymphoblastic leukemia (BCP-ALL), B cell lymphoma, hairy cell leukemia, acute lymphocytic leukemia (ALL), and Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, and ureter cancer. Preferably, the cancer is a hematological malignancy (e.g., leukemia or lymphoma, including but not limited to Hodgkin lymphoma, non-Hodgkin lymphoma, CLL, acute lymphocytic cancer, acute myeloid leukemia (AML), B-chronic lymphocytic leukemia, hairy cell leukemia, acute lymphocytic leukemia (ALL) (also referred to as “acute lymphoblastic leukemia”), B-ALL, BCP-ALL, B cell lymphoma, and Burkitt's lymphoma). Preferably, the cancer is characterized by the expression of CD123.

In some embodiments, the disease, disorder, or condition is a hematologic malignancy, or a cancer of the blood. In some embodiments, the malignancy is a lymphoid malignancy or a myeloid malignancy. In some embodiments, the disease, disorder, or condition is a hematopoietic malignancy. In some embodiments, the disease, disorder, or condition is a leukemia, e.g., acute myeloid leukemia (AML). AML is characterized as a heterogeneous, clonal, neoplastic disease that originates from transformed cells that have progressively acquired critical genetic changes that disrupt key differentiation and growth-regulatory pathways. (Dohner et al., NEJM, (2015) 373:1136). Without wishing to be bound by theory, it is believed in some embodiments, that CD123 is expressed on myeloid leukemia cells as well as on normal myeloid and monocytic precursors and is an attractive target for AML therapy.

In some embodiments, the hematopoietic malignancy or hematological disorder associated with CD123 is a precancerous condition such as a myelodysplasia, a myelodysplastic syndrome or a preleukemia. Myelodysplastic syndromes (MDS) are hematological medical conditions characterized by disorderly and ineffective hematopoiesis, or blood production. Thus, the number and quality of blood-forming cells decline irreversibly. Some patients with MDS can develop severe anemia, while others are asymptomatic. The classification scheme for MDS is known in the art, with criteria designating the ratio or frequency of particular blood cell types, e.g., myeloblasts, monocytes, and red cell precursors. MDS includes refractory anemia, refractory anemia with ring sideroblasts, refractory anemia with excess blasts, refractory anemia with excess blasts in transformation, chronic myelomonocytic leukemia (CML). In some embodiments, MDS can progress to an acute myeloid leukemia (AML).

Furthermore, the treatment or prevention provided by the methods described herein can include treatment or prevention of one or more conditions or symptoms of the disease being treated or prevented.

Aspects of the present disclosure also provide methods of detecting the presence of a disease, disorder, or condition in a subject, comprising: (a) contacting a sample comprising one or more cells from the subject with any of the CARs, nucleic acids, vectors, cells expressing any of the CARs, populations of such cells, or any of the pharmaceutical compositions described herein, thereby forming a complex (e.g., between the CAR and the target antigen (i.e., CD123)), (b) and detecting the complex, wherein detection of the complex is indicative of the presence of the disease, disorder, or condition in the subject.

The sample may be obtained by any suitable method, e.g., biopsy or necropsy. A biopsy is the removal of tissue and/or cells from an individual. Such removal may be to collect tissue and/or cells from the individual in order to perform experimentation on the removed tissue and/or cells. This experimentation may include experiments to determine if the individual has and/or is suffering from a certain condition or disease-state. The condition or disease may be, e.g., cancer, a hematopoictic malignancy or pre-malignancy.

In some embodiments, the sample comprising cells of the subject can be a sample comprising whole cells, lysates thereof, or a fraction of the whole cell lysates, e.g., a nuclear or cytoplasmic fraction, a whole protein fraction, or a nucleic acid fraction. If the sample comprises whole cells, the cells can be any cells of the subject, e.g., the cells of any organ or tissue, including blood cells or endothelial cells.

In some embodiments, the contacting of the sample with any of the CARs, nucleic acids, vectors, cells expressing any of the CARs, populations of such cells, or any of the pharmaceutical compositions described herein, can take place in vitro or in vivo with respect to the subject. Preferably, the contacting is in vitro.

Detection of the complex can occur through any number of ways known in the art. For instance, any of the CARs, nucleic acids, vectors, cells expressing any of the CARs, populations of such cells, or any of the pharmaceutical compositions described herein, can be labeled with a detectable label such as, for instance, a radioisotope, a fluorophore (e.g., fluorescein isothiocyanate (FITC), phycocrythrin (PE)), an enzyme (e.g., alkaline phosphatase, horseradish peroxidase), and element particles (e.g., gold particles).

Methods of testing a CAR for the ability to recognize target cells and for antigen specificity are known in the art. For instance, Clay et al., J. Immunol (1999) 163:507-513, teaches methods of measuring the release of cytokines (e.g., interferon-gamma, granulocyte/monocyte colony stimulating factor (GM-CSF), tumor necrosis factor alpha (TNF-a) or interleukin 2 (IL-2)). In addition, CAR function can be evaluated by measurement of cellular cytotoxicity, as described in Zhao et al., J. Immunol. (2005) 174:4415-4423.

### Hematopoietic Cells Deficient in CD123

Aspects of the present disclosure provide compositions and methods for the inhibition of a CD123 target antigen. Such treatment regimen can involve, for example, the following steps: (1) administering a therapeutically effective amount of a cell or population of cells (e.g., immune cells, e.g., T lymphocytes, NK cells) to the patient, where the cell comprises a nucleic acid sequence encoding any of the chimeric antigen receptors (CARs) targeting CD123 described herein; and (2) administering (e.g., infusing or reinfusing) the patient with hematopoietic stem cells, either autologous or allogeneic, where the hematopoietic cells have reduced expression of CD123. In some embodiments, the hematopoietic cells are genetically modified to have reduced or eliminated expression of CD123.

In some embodiments, the hematopoietic cells are hematopoietic stem cells (HSCs). In some embodiments, the hematopoietic cells are hematopoietic progenitor cells (HPCs). Hematopoictic stem cells (HSCs) are capable of giving rise to both myeloid and lymphoid progenitor cells that further give rise to myeloid cells (e.g., monocytes, macrophages, neutrophils, basophils, dendritic cells, erythrocytes, platelets, etc) and lymphoid cells (e.g., T cells, B cells, NK cells), respectively. HSCs are characterized by the expression of the cell surface marker CD34 (e.g., CD34+), which can be used for the identification and/or isolation of HSCs, and absence of cell surface markers associated with commitment to a cell lineage. In some embodiments, the HSCs are peripheral blood HSCs.

In some embodiments, the hematopoietic cells are obtained from a subject, such as a mammalian subject. In some embodiments, the mammalian subject is a non-human primate, a rodent (e.g., mouse or rat), a bovine, a porcine, an equine, or a domestic animal. In some embodiments, the hematopoietic cells are obtained from a human patient, such as a human patient having a hematopoietic malignancy or pre-malignancy. In some embodiments, the hematopoietic cells are obtained from a healthy donor. In some embodiments, the hematopoietic cells are obtained from the subject to whom the immune cells expressing the chimeric antigen receptors will be subsequently administered.

HSCs may be obtained from any suitable source using convention means known in the art. In some embodiments, HSCs are obtained from a sample from a subject, such as bone marrow sample or from a blood sample. Alternatively or in addition, HSCs may be obtained from an umbilical cord. In some embodiments, the HSCs are from bone marrow or peripheral blood mononuclear cells (PBMCs). In general, bone marrow cells may be obtained from iliac crest, femora, tibiae, spine, rib or other medullary spaces of a subject. Bone marrow may be taken out of the patient and isolated through various separations and washing procedures known in the art. An exemplary procedure for isolation of bone marrow cells comprises the following steps: a) extraction of a bone marrow sample; b) centrifugal separation of bone marrow suspension in three fractions and collecting the intermediate fraction, or buffycoat; c) the buffycoat fraction from step (b) is centrifuged one more time in a separation fluid, commonly Ficoll™, and an intermediate fraction which contains the bone marrow cells is collected; and d) washing of the collected fraction from step (c) for recovery of re-transfusable bone marrow cells. Methods of obtaining mammalian cells, such as hematopoietic stem cells, are described, e.g., in PCT Application No. US2016/057339, which is herein incorporated by reference in its entirety.

HSCs typically reside in the bone marrow but can be mobilized into the circulating blood by administering a mobilizing agent in order to harvest HSCs from the peripheral blood. In some embodiments, the subject from which the HSCs are obtained is administered a mobilizing agent, such as granulocyte colony-stimulating factor (G-CSF). The number of the HSCs collected following mobilization using a mobilizing agent is typically greater than the number of cells obtained without use of a mobilizing agent.

In some embodiments, a sample is obtained from a subject and is then enriched for a desired cell type (e.g., CD34+/CD33-cells). For example, PBMCs and/or CD34+ hematopoietic cells can be isolated from blood as described herein. Cells can also be isolated from other cells, for example by isolation and/or activation with an antibody binding to an epitope on the cell surface of the desired cell type. Another exemplary method that can be used includes negative selection using antibodies to cell surface markers to selectively enrich for a specific cell type without activating the cell by receptor engagement.

Populations of HSC can be expanded prior to or after genetically engineering the HSC to become deficient in a target antigen (i.e., CD123). The cells may be cultured under conditions that comprise an expansion medium comprising one or more cytokines, such as stem cell factor (SCF), Flt-3 ligand (FLt3L), thrombopoietin (TPO), Interleukin 3 (IL-3), or Interleukin 6 (IL-6). The cell may be expanded for about 1, 2, 3, 4, 5, 6, 7, 8, 9, 10, 11, 12, 13, 14, 15, 16, 17, 18, 19, 20, 21, 22, 23, 23, 25 days or any range necessary. In some embodiments, HSCs are expanded after isolation of a desired cell population (e.g., CD34+/CD33-) from a sample obtained from a subject and prior to genetic engineering. In some embodiments, the HSC are expanded after genetic engineering, thereby selectively expanding cells that have undergone the genetic modification and are deficient in a lineage-specific cell-surface antigen. In some embodiments, a cell (“a clone”) or several cells having a desired characteristic (e.g., phenotype or genotype) following genetic modification may be selected and independently expanded.

In some embodiments, the hematopoietic cells are genetically engineered to be deficient in a target antigen, such as a cancer antigen or an antigen associated with the disease, disorder, or condition. In some embodiments, the hematopoietic cells are genetically engineered to be deficient in the same target antigen that is targeted by the CARs described herein. In some embodiments, the hematopoietic cells are genetically engineered to be deficient in CD123. In some embodiments, the hematopoietic cells are genetically engineered to be deficient in a subunit of CD123, such as the alpha subunit or the beta subunit. In some embodiments, the hematopoietic cells are genetically engineered to be deficient in the alpha subunit of CD123. In some embodiments, the hematopoietic cells are genetically engineered to be deficient in the beta subunit of CD123. In some embodiments, the hematopoietic cells are genetically engineered to be deficient in both the alpha and beta subunits of CD123. As used herein, a hematopoietic cell is considered to be deficient in a target antigen if the hematopoietic cell has substantially reduced expression of the target antigen as compared to a naturally-occurring hematopoictic cell of the same type as the genetically engineered hematopoietic cell (e.g., is characterized by the presence of the same cell surface markers, such as CD34). In some embodiments, the hematopoietic cell has no detectable expression of the target antigen. The expression level of a target antigen can be assessed by any means known in the art. For example, the expression level of a target antigen can be assessed by detecting the antigen with an antigen-specific antibody (e.g., flow cytometry methods, Western blotting) and/or by measuring the level of a transcript encoding the antigen (e.g., RT-qPCR, microarray).

In some embodiments, the expression of the target antigen on the genetically engineered hematopoietic cell is compared to the expression of the target antigen on a naturally occurring hematopoietic cell. In some embodiments, the genetic engineering results in a reduction in the expression level of the target antigen by at least about 50%, 60%, 70%, 80%, 90%, 91%, 92%, 93%, 94%, 95%, 96%, 97%, 98%, or 99% as compared to the expression of the target antigen on a naturally occurring hematopoietic cell.

In some embodiments, the hematopoietic cell is deficient in the whole endogenous gene encoding the target antigen. In some embodiments, the whole endogenous gene encoding the target antigen has been deleted. In some embodiments, the hematopoietic cell comprises a portion of endogenous gene encoding the target antigen. In some embodiments, the hematopoictic cell expressing a portion (e.g. a truncated protein) of the target antigen. In other embodiments, a portion of the endogenous gene encoding the target antigen has been deleted. In some embodiments, at least 10%, 20%, 30%, 40%, 50%, 60%, 70% or more of the gene encoding the target antigen has been deleted.

As will be appreciated by one of ordinary skill in the art, a portion of the nucleotide sequence encoding the target antigen may be deleted or one or more non-coding sequences, such that the hematopoietic cell is deficient in the antigen (e.g., has substantially reduced expression of the antigen).

In some embodiments, the target antigen is CD123. The predicted structure of CD123 includes an alpha subunit that binds to a ligand (e.g., IL-3) and a beta subunit that transduces the signal. In some embodiments, the alpha subunit (or portion thereof) of CD123 is deleted. In some embodiments, the beta subunit (or portion thereof) of CD123 is deleted. In some embodiments, both the alpha subunit and beta subunit of CD123 are deleted.

Any of the genetically engineering hematopoietic cells, such as HSCs, that are deficient in a target antigen can be prepared by routine methods or by methods described herein. In some embodiments, the genetic engineering is performed using genome editing. As used herein, “genome editing” refers to a method of modifying the genome, including any protein-coding or non-coding nucleotide sequence, of an organism to knock-out the expression of a target gene. In general, genome editing methods involve use of an endonuclease that is capable of cleaving the nucleic acid of the genome, for example at a targeted nucleotide sequence. Repair of the double-stranded breaks in the genome may be repaired introducing mutations and/or exogenous nucleic acid may be inserted into the targeted site.

Genome editing methods are generally classified based on the type of endonuclease that is involved in generating double stranded breaks in the target nucleic acid. These methods include use of zinc finger nucleases (ZFN), transcription activator-like effector-based nuclease (TALEN), meganucleases, and CRISPR/Cas systems. Methods of editing the genome of HSCs described herein can be found, e.g., in PCT Publication No. WO 2017/066760, incorporated by reference herein.

### Combination Therapy

As described herein, any of the CARs that target CD123, nucleic acids, vectors, cells expressing any of the CARs, and/or pharmaceutical compositions described herein may be administered to a subject in combination with hematopoietic cells that are deficient for the target antigen (i.e., CD123).

In some embodiments, the agents and/or the hematopoietic cells may be mixed with a pharmaceutically acceptable carrier to form a pharmaceutical composition, which is also within the scope of the present disclosure.

To perform the methods described herein, an effective amount of any of the CARs that target CD123, nucleic acids, vectors, cells expressing any of the CARs, and/or pharmaceutical compositions described herein and an effective amount of hematopoietic cells can be co-administered to a subject in need of the treatment.

As described herein, the hematopoietic cells and/or cells expressing chimeric antigen receptors (e.g., immune cells) may be autologous to the subject. i.e., the cells are obtained from the subject in need of the treatment, genetically engineered to be deficient for expression of target antigen or for expression of the chimeric antigen receptor, and then administered to the same subject. Administration of autologous cells to a subject may result in reduced rejection of the cells as compared to administration of non-autologous cells. Alternatively, the hematopoietic cells and/or cells expressing chimeric antigen receptors (e.g., immune cells) are allogeneic cells, i.e., the cells are obtained from a first subject, genetically engineered to be deficient for expression of the target antigen or for expression of the chimeric antigen receptor, and administered to a second subject that is different from the first subject but of the same species. For example, allogeneic immune cells may be derived from a human donor (e.g., a healthy donor) and administered to a human recipient who is different from the donor.

In some embodiments, the cells (e.g., immune cells) expressing any of the CARs described herein and/or hematopoietic cells are allogeneic cells and have been further genetically engineered to reduced graft-versus-host disease. For example, as described herein, the hematopoietic stem cells may be genetically engineered (e.g., using genome editing) to have reduced expression of CD45RA.

In some embodiments, the cells (e.g., immune cells) expressing any of the CARs described herein are administered to a subject in an amount effective in to reduce the number of target cells (e.g., cancer cells, malignant cells) by least 20%, e.g., 50%, 80%, 100%, 2-fold, 5-fold, 10-fold, 20-fold, 50-fold, 100-fold or more.

A typical amount of cells, i.e., cells (e.g., immune cells) expressing any of the CARs described herein or hematopoietic cells, administered to a mammal (e.g., a human) can be, for example, in the range of one million to 100 billion cells; however, amounts below or above this exemplary range are also within the scope of the present disclosure. For example, the daily dose of cells can be about 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), preferably about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), more preferably about 100 million cells to about 50 billion cells (e.g., about 120 million cells, about 350 million cells, about 350 million cells, about 450 million cells, about 650 million cells, about 800 million cells, about 900 million cells, about 3 billion cells, about 30 billion cells, about 45 billion cells, or a range defined by any two of the foregoing values).

In one embodiment, a CAR (e.g., a nucleic acid encoding the chimeric receptor) is introduced into a cell (e.g., an immune cell), and the subject (e.g., human patient) receives an initial administration or dose of the cells expressing the CAR. One or more subsequent administrations of the cells (e.g., immune cells) expressing the CAR may be provided to the patient at intervals of 15 days, 14, 13, 12, 11, 10, 9, 8, 7, 6, 5, 4, 3, or 2 days after the previous administration. More than one dose of the cells (e.g., immune cells) expressing the CAR can be administered to the subject per week, e.g., 2, 3, 4, or more administrations of the cells. The subject may receive more than one doses of the cells (e.g., immune cells) expressing the CAR per week, followed by a week of no administration of the cells, and finally followed by one or more additional doses of the cells (e.g., immune cells) expressing the CAR (e.g., more than one administration of the cells per week). The cells (e.g., immune cells) expressing the CAR may be administered every other day for 3 administrations per week for two, three, four, five, six, seven, eight or more weeks.

In some embodiments, the methods involve administration of cells (e.g., immune cells) expressing the CAR targeting CD123 and a population of hematopoietic cells deficient in the target antigen (i.e., CD123). Accordingly, in such therapeutic methods, the CAR recognizes (binds) a target cell expressing the target antigen for targeting killing. The hematopoietic cells that are deficient in the target antigen allow for repopulation of a cell type that is targeted by the cells/CARs. In some embodiments, the treatment of the patient can involve the following steps: (1) administering a therapeutically effective amount of cells (e.g., immune cells) expressing the CAR targeting CD123 to the patient and (2) infusing or reinfusing the patient with hematopoietic stem cells, either autologous or allogenic, where the hematopoietic cells have reduced expression of a target antigen (i.e., CD123). In some embodiments, the treatment of the patient can involve the following steps: (1) administering a therapeutically effective amount of cells (e.g., immune cells) expressing the CAR targeting CD123, wherein the cell comprises a nucleic acid sequence encoding a chimeric antigen receptor that binds a cell-surface lineage-specific, disease-associated antigen (i.e., CD123); and (2) infusing or reinfusing the patient with hematopoietic cells (e.g., hematopoietic stem cells), either autologous or allogenic, where the hematopoietic cells have reduced expression of a lineage specific disease-associated antigen (i.e., CD123).

The efficacy of the therapeutic methods using any of the CARs, nucleic acids, vectors, cells expressing any of the CARs, or pharmaceutical compositions comprising any of the foregoing described herein, and a population of hematopoietic cells deficient in the target antigen may be assessed by any method known in the art and would be evident to a skilled medical professional. For example, the efficacy of the therapy may be assessed by survival of the subject or cancer burden in the subject or tissue or sample thereof. In some embodiments, the efficacy of the therapy is assessed by quantifying the number of cells belonging to a particular population or lineage of cells. In some embodiments, the efficacy of the therapy is assessed by quantifying the number of cells presenting the target antigen (i.e., CD123).

In some embodiments, any of the CARs, nucleic acids, vectors, cells expressing any of the CARs, or pharmaceutical compositions comprising any of the foregoing described herein and the population of hematopoietic cells is administered concomitantly.

In some embodiments, any of the CARs, nucleic acids, vectors, cells expressing any of the CARs, or pharmaceutical compositions comprising any of the foregoing described herein are administered prior to administration of the hematopoietic cells. In some embodiments, any of the CARs, nucleic acids, vectors, cells expressing any of the CARs, or pharmaceutical compositions comprising any of the foregoing described herein are administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of the hematopoietic cells.

In some embodiments, the hematopoietic cells are administered prior to the any of the CARs, nucleic acids, vectors, cells expressing any of the CARs, or pharmaceutical compositions comprising any of the foregoing described herein. In some embodiments, the population of hematopoietic cells is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of any of the CARs, nucleic acids, vectors, cells expressing any of the CARs, or pharmaceutical compositions comprising any of the foregoing described herein.

In some embodiments, any of the CARs, nucleic acids, vectors, cells expressing any of the CARs, or pharmaceutical compositions comprising any of the foregoing described herein and the population of hematopoietic cells are administered at substantially the same time. In some embodiments, any of the CARs, nucleic acids, vectors, cells expressing any of the CARs, or pharmaceutical compositions comprising any of the foregoing described herein is administered and the patient is assessed for a period of time, the population of hematopoietic cells is administered and the patient is assessed for a period of time, after which any of the CARs, nucleic acids, vectors, cells expressing any of the CARS, or pharmaceutical compositions comprising any of the foregoing described herein is administered.

Also within the scope of the present disclosure are multiple administrations (e.g., doses) of any of the CARs, nucleic acids, vectors, cells expressing any of the CARs, or pharmaceutical compositions comprising any of the foregoing described herein and/or populations of hematopoietic cells. In some embodiments, any of the CARs, nucleic acids, vectors, cells expressing any of the CARs, or pharmaceutical compositions comprising any of the foregoing described herein and/or populations of hematopoietic cells are administered to the subject once. In some embodiments, any of the CARs, nucleic acids, vectors, cells expressing any of the CARs, or pharmaceutical compositions comprising any of the foregoing described herein and/or populations of hematopoietic cells are administered to the subject more than once (e.g., at least 2, 3, 4, 5, or more times). In some embodiments, any of the CARs, nucleic acids, vectors, cells expressing any of the CARs, or pharmaceutical compositions comprising any of the foregoing described herein and/or populations of hematopoietic cells are administered to the subject at a regular interval, e.g., every six months.

In some embodiments, the subject is a human subject having a hematopoictic malignancy or pre-malignancy. In some embodiments, the subject is a human subject that has been diagnosed with a hematopoietic malignancy or pre-malignancy. As used herein a hematopoietic malignancy refers to a malignant abnormality involving hematopoictic cells (e.g., blood cells, including progenitor and stem cells). Examples of hematopoietic malignancies and or pre-malignancies include, without limitation, Hodgkin's lymphoma, non-Hodgkin's lymphoma, leukemia, or multiple myeloma. Leukemias include acute myeloid leukemia (AML), myelodysplastic syndrome (MDS), chronic myelogenous leukemia, chronic lymphoblastic leukemia, and chronic lymphoid leukemia. In some embodiments, the hematopoictic malignancy is acute myeloid leukemia (AML). In some embodiments, the hematopoictic malignancy is myelodysplastic syndrome (MDS).

### Kits for Therapeutic Uses

Also within the scope of the present disclosure are kits for use of any of the CARs, nucleic acids, vectors, and/or cells expressing any of the CARs described herein in combination with populations of hematopoietic cells that are deficient in a target antigen (i.e., CD123), or portion thereof. Such kits may include one or more containers comprising a first pharmaceutical composition that comprises any of the CARs, nucleic acids, vectors, and/or cells expressing any of the CARs described herein and a pharmaceutically acceptable carrier, and a second pharmaceutical composition that comprises a population of hematopoietic cells that are deficient in a target antigen (i.e., CD123), or portion thereof and a pharmaceutically acceptable carrier.

In some embodiments, the kit can comprise instructions for use in any of the methods described herein. The included instructions can comprise a description of administration of the first and second pharmaceutical compositions to a subject to achieve the intended activity in a subject. The kit may further comprise a description of selecting a subject suitable for treatment based on identifying whether the subject is in need of the treatment. In some embodiments, the instructions comprise a description of administering the first and second pharmaceutical compositions to a subject who is in need of the treatment.

The instructions relating to the use of the CARs, nucleic acids, vectors, and/or cells expressing any of the CARs described herein and the first and second pharmaceutical compositions described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a chimeric receptor variants as described herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.): Handbook of Experimental Immunology (D. M. Weir and C. C. Blackwell, eds.): Gene Transfer Vectors for Mammalian Cells (J. M. Miller and M. P. Calos, eds., 1987; Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, cd. (1986»; Immobilized Cells and Enzymes (IRL Press, (1986>; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Examples

### Example 1. Specific Activation and Cytotoxic Targeting of CD123+ Cells

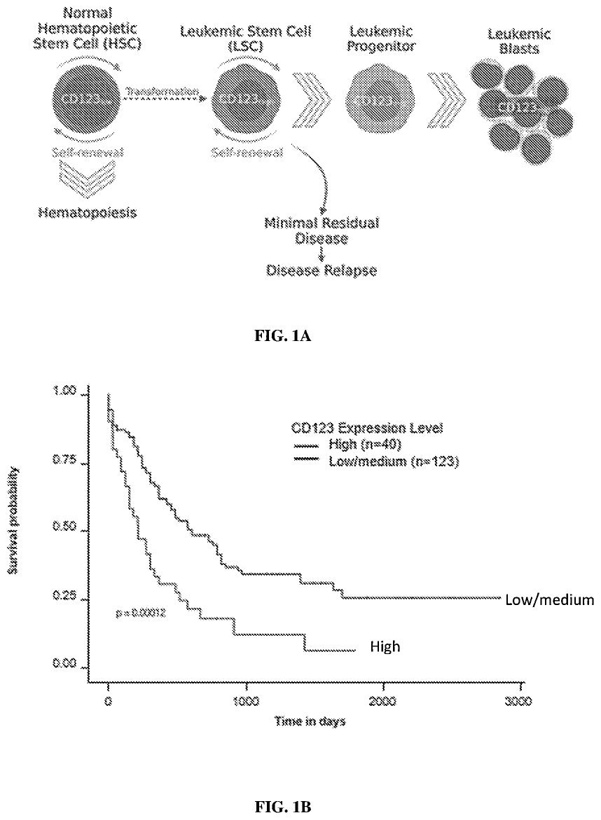

Acute myeloid leukemia (AML) is an aggressive bone marrow malignancy, characterized by the presence of leukemic blasts in the peripheral blood of patients. Poor AML prognoses¹ are largely attributable to high rates of disease relapse, of which CD123+ leukemic stem cells (LSCs) are the primary cause (FIG. 1A).^{2,3} CD123, the alpha-chain of the IL3 cytokine receptor,⁶ has been identified as a favorable therapeutic AML target, overexpressed in both LSCs and blasts.^{4,5} High CD123 expression levels in AML patients has also been correlated with significantly reduced survival, as compared to patients having low or medium levels of CD123 expression (FIG. 1B).

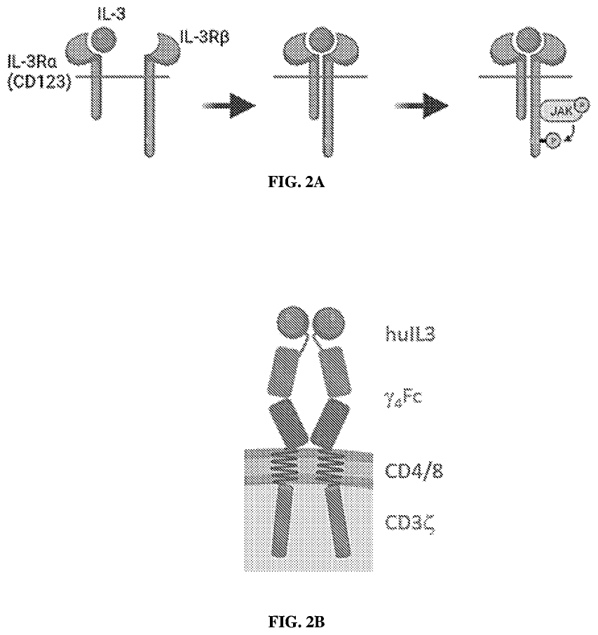

T cells were directed to CD123+AML cells via a cell surface-tethered IL-3 chimeric antigen receptor (CAR) (termed “IL3-zetakine”). Without wishing to be bound by any particular theory, it is thought that upon binding of CD123 (also referred to as IL-3Rα) to the IL3 receptor beta chain (IL-3Rβ), dimerization of these two factors forms a complex that initiates the Janus kinase (JAK)/signal transducer and activator of transcription pathway (FIG. 2A). In contrast to a conventional chimeric antigen receptor (CAR) construct that comprises an antigen-binding domain of an antibody to direct the CAR to the target antigen, the zetakines described herein enable structure-guided site-directed mutagenesis to increase binding affinity and alter target cell signaling without determinantal T cell hyperactivation.

### Methods

Exemplary zetakine constructs were designed using IL3 sequences (SEQ ID NOs: 1-5) linked to a transmembrane domain (e.g., SEQ ID NOs: 12-14) and a CD3z signaling domain (e.g., SEQ ID NOs: 15 and 16) (FIG. 2B). Constructs were optionally designed to include one or more of the following: intracellular costimulatory signaling domain (e.g., SEQ ID NOs: 17 and 18), a linker region, and/or a hinge region (FIG. 2B). Exemplary zetakine constructs are listed in Table 1.

TABLE 1
Exemplary zetakine constructs

| | | | | | | TIB153 % CD69 | |
|---|---|---|---|---|---|---|---|
| | | | | | TIB153 | activation: | |
| | | | | | Transduction | MOLM-13-WT/ | PBMC |
| | | | | | Efficiency | MOLM-13 | Transduction |
| Construct | Cytokine | Hinge | Transmembrane | Costimulatory | (anti-IL3 AB) | CD123KO | Efficiency |
| IL3-WT-nocostim | IL3 | IgG4 [SmP] | CD4 | None | 39.8 | 6.39 | 18.98 |
| (SEQ ID NO: 21) | (SEQ ID NO: 2) | (SEQ ID NO: 10) | (SEQ ID NO: 14) | | | | |
| IL13-K110E- | IL3-K110E* | IgG4 [SmP] | CD4 | None | 42.5 | 1.06 | — |
| nocostim | (SEQ ID NO: 3) | (SEQ ID NO: 10) | (SEQ ID NO: 14) | | | | |
| (SEQ ID NO: 27) | | | | | | | |
| IL13-WT- | IL3 | IgG4 [SmP] | CD4 | 41BB | 2.06 | 4.74 | 4.34 |
| 41BBcostim (SEQ | (SEQ ID NO: 2) | (SEQ ID NO: 10) | (SEQ ID NO: 14) | (SEQ ID NO: 17) | | | |
| ID NO: 25) | | | | | | | |
| IL13-K110E- | IL3-K110E* | IgG4 [SmP] | CD4 | 41BB | 4.15 | 1.28 | — |
| 41BBcostim | (SEQ ID NO: 3) | (SEQ ID NO: 10) | (SEQ ID NO: 14) | (SEQ ID NO: 17) | | | |
| (SEQ ID NO: 29) | | | | | | | |
| IL13-WT- | IL3 | IgG4 [SmP] | CD4 | CD28 | 0 | — | — |
| CD28costim | (SEQ ID NO: 2) | (SEQ ID NO: 10) | (SEQ ID NO: 14) | (SEQ ID NO: 17) | | | |
| (SEQ ID NO: 23) | | | | | | | |
| IL13-K110E- | IL3-K110E* | IgG4 [SmP] | CD4 | CD28 | 3.6 | 1.03 | — |
| CD28costim | (SEQ ID NO: 3) | (SEQ ID NO: 10) | (SEQ ID NO: 14) | (SEQ ID NO: 17) | | | |
| (SEQ ID NO: 29) | | | | | | | |
| IL3-WT-no | IL3 | IgG4 [SmP] | CD8 | None | 94.96 | — | — |
| costim + CD8 | (SEQ ID NO: 2) | (SEQ ID NO: 10) | (SEQ ID NO: 12) | | | | |
| transmembrane | | | | | | | |
| (SEQ ID NO: 33) | | | | | | | |
| IL3-WT-no | IL3 | IgG4-L235E, | CD4 | None | 67.67 | — | — |
| costim + IgG4 | (SEQ ID NO: 2) | N297Q | (SEQ ID NO: 14) | | | | |
| L235E, N297Q | | (SEQ ID NO: 11) | | | | | |
| hinge | | | | | | | |
| (SEQ ID NO: 35) | | | | | | | |
| IL3-WT-no | IL3 | CD8 | CD4 | None | 77.36 | — | — |
| costim + CD8 | (SEQ ID NO: 2) | (SEQ ID NO: 8) | (SEQ ID NO: 14) | | | | |
| hinge | | | | | | | |
| (SEQ ID NO: 37) | | | | | | | |
| IL3-D101A, | IL3-D101A, | IgG4 [SmP] | CD4 | None | 92.72 | — | — |
| K116V-no costim | K116V | (SEQ ID NO: 10) | (SEQ ID NO: 14) | | | | |
| (SEQ ID NO: 39) | (SEQ ID NO: 4) | | | | | | |
| IL3-D101A, | IL3-D101A, | IgG4 [SmP] | CD4 | 41BB | 16.7 | — | — |
| K116V-41BB | K116V | (SEQ ID NO: 10) | (SEQ ID NO: 14) | (SEQ ID NO: 17) | | | |
| costim | (SEQ ID NO: 4) | | | | | | |
| (SEQ ID NO: 41) | | | | | | | |
| IL3-D101A, | IL3-D101A, | IgG4 [SmP] | CD4 | CD28 | 1.15 | — | — |
| K116V-CD28 | K116V | (SEQ ID NO: 10) | (SEQ ID NO: 14) | (SEQ ID NO: 18) | | | |
| costim | (SEQ ID NO: 4) | | | | | | |
| (SEQ ID NO: 43) | | | | | | | |
| IL3-D101A, | IL3-D101A, | IgG4 [SmP] | CD8 | None | 95.57 | — | — |
| K116V-no | K116V | (SEQ ID NO: 10) | (SEQ ID NO: 12) | | | | |
| costim + CD8 | (SEQ ID NO: 4) | | | | | | |
| transmembrane | | | | | | | |
| (SEQ ID NO: 45) | | | | | | | |
| IL3-D101A, | IL3-D101A, | IgG4- | CD4 | None | 54.11 | — | — |
| K116V-no | K116V | L235E, N297Q | (SEQ ID NO: 14) | | | | |
| costim + IgG4 | (SEQ ID NO: 4) | (SEQ ID NO: 11) | | | | | |
| L235E, N297Q | | | | | | | |
| hinge | | | | | | | |
| (SEQ ID NO: 47) | | | | | | | |
| IL3-D101A, | IL3-D101A, | CD8 | CD4 | None | 28.39 | — | — |
| K116V-no | K116V | (SEQ ID NO: 8) | (SEQ ID NO: 14) | | | | |
| costim + CD8 | (SEQ ID NO: 4) | | | | | | |
| hinge | | | | | | | |
| (SEQ ID NO: 49) | | | | | | | |
| IL3-K116W- | IL3-K116W | IgG4 [SmP] | CD4 | None | — | — | — |
| nocostim | (SEQ ID NO: 5) | (SEQ ID NO: 11) | (SEQ ID NO: 14) | | | | |
| (SEQ ID NO: 51) | | | | | | | |

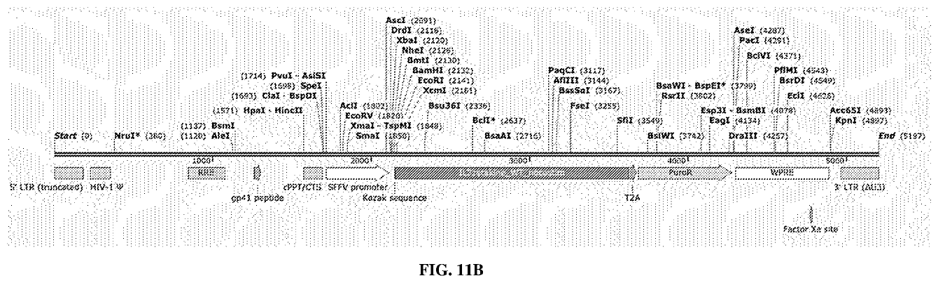

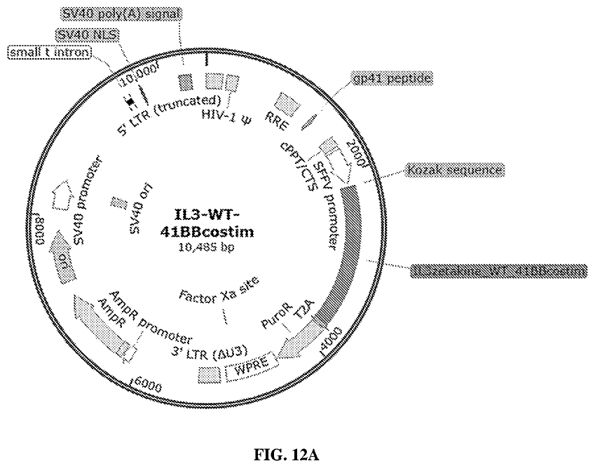

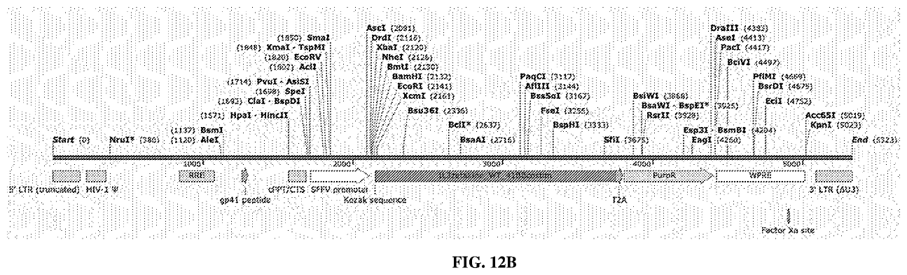

Exemplary vectors encoding IL3-zetakines are shown in FIGS. 11A-12B. An exemplary nucleotide sequence for the construct shown in FIG. 11B is provided by SEQ ID NO: 54. An exemplary nucleotide sequence for the construct shown in FIG. 12B is provided by SEQ ID NO: 55.

The constructs were transduced into TIB-153™ cells (Jurkat cells that lack the beta chain of the TCR receptor) using lentiviral vectors (LVV). To detect expression of IL3 zetakines, cells were stained with anti-IL3 antibodies (aIL3) or biotinylated CD123 protein (rCD123) bound to fluorescence-tagged streptavidin. Expression was quantified by flow cytometry 7 days following transduction.

T cell activation was assessed by measuring CD69 expression via flow cytometry of sorted IL3-zetakine-positive TIB-153™ cells after co-culture with MOLM13 AML cells (which express CD123). Constructs were selected for further analysis based on initial transduction percentage and activation response.

### Results

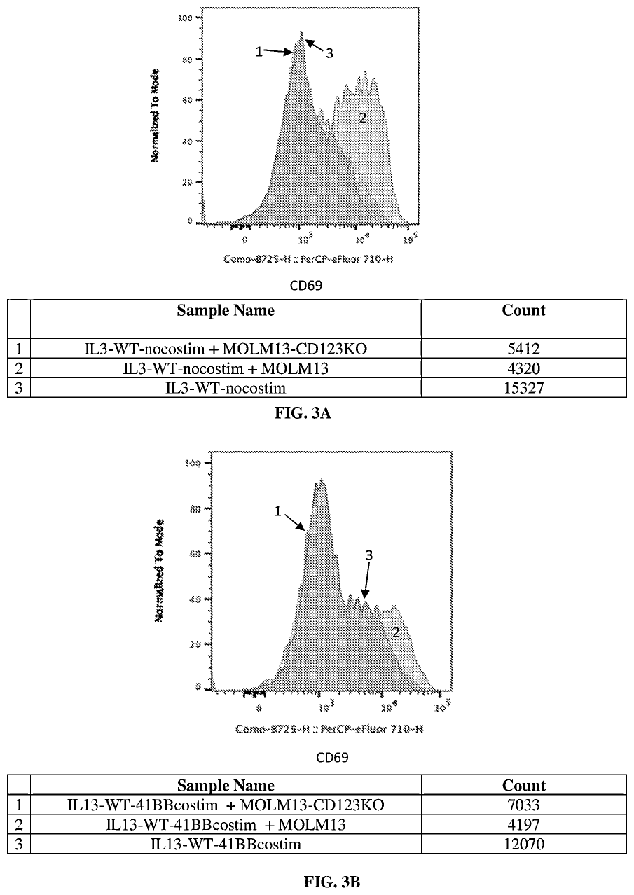

Transduction of the exemplary zetakine constructs (Table 1) yielded a range of transduction percentages in TIB-153™ cells (0-98%) prior to cell sorting. In co-culture with CD123+MOLM13 AML cells, TIB-153™ cells expressing CARs containing wildtype IL-3 (or a wildtype fragment thereof) lacking a costimulatory domain were found to induce the highest level of CD69 expression of the constructs tested. Co-culture of TIB-153™ cells expressing the IL3-WT-nocostim construct (SEQ ID NO: 21) with MOLM13 cells resulted in increased CD69 activation relative to TB-153TM cells expressing the IL3-zetakine alone or in co-culture with MOL13 cells deficient in CD123 (MOLM13-CD123KO) (FIGS. 3A and 3B).

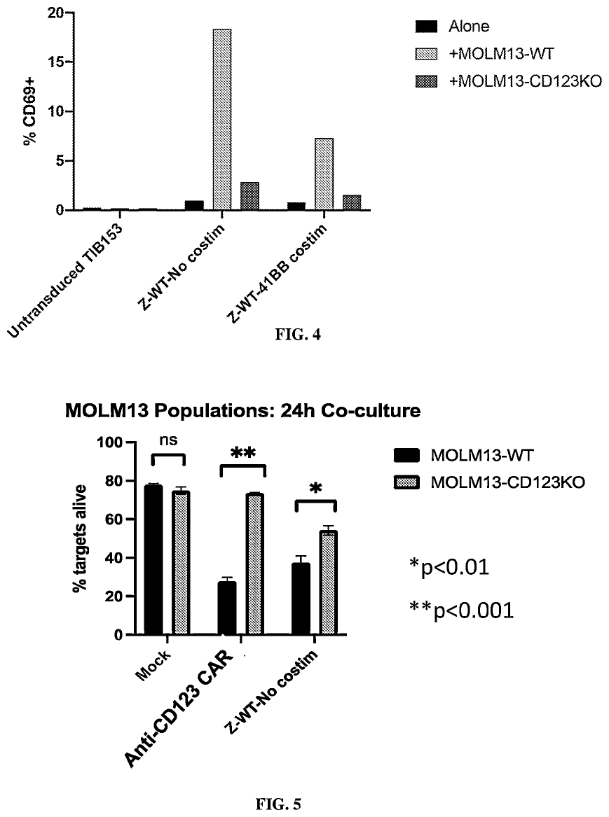

Further, induction of CD69 expression in TB-153™ cells expressing a IL3-zetakine was shown to occur in an antigen-specific manner. Co-cultures of TB-153TM cells expressing the IL3-WT-nocostim (SEQ ID NO:21) construct with MOLM13 cells showed an average of 18.7% CD69+ T cells and a 5.3-fold increase of CD69+ T cells over cells cultured with MOLM13-CD123KO (FIG. 4, Table 2).

Interestingly, cells expressing an IL3-zetakine construct containing a 4-1BB costimulatory signaling domain (SEQ ID NO: 23) showed lower levels of CD69 expression as compared to cells expressing IL3-zetakine constructs that lacked the costimulatory domain (FIG. 3B). The percent CD69+ cells in each experimental condition are quantified in FIG. 4.

TABLE 2
CD69 activation of zetakine constructs co-cultured with
MOLM13 or MOLM13-CD123KO

| | MOLM13 | MOLM13 | Average |
|---|---|---|---|
| | fold change | fold change | MOLM13-WT |
| | over MOLM- | over MOLM- | fold change |
| | CD123KO, | CD123KO, | over MOLM- |
| Zetakine construct | Replicate 1 | Replicate 2 | CD123KO |
| IL3-WT-nocostim | 6.40 | 4.20 | 5.3 |
| (SEQ ID NO: 21) | | | |
| IL13-WT- | 4.74 | 2.94 | 3.84 |
| 41BBcostim | | | |
| (SEQ ID NO: 23) | | | |

A K110E mutant of IL-3 was previously reported to exhibit a 40-fold increased affinity over wildtype⁸, however cells expressing a IL3-zetakine containing the corresponding K110E mutation (SEQ ID NO: 27) showed lower zetakine function as compared to the wildtype IL3 zetakine (Table 1).

Finally, cells expressing IL3-zetakine constructs were assessed for their ability to induce cytotoxicity of CD123-expressing target cells. As a positive control, cells were generated to express conventional anti-CD123 CAR construct containing an anti-CD123 binding domain from an antibody (see, PCT Publication No. WO 2015/140268 A1).

Exemplary conventional anti-CD123 CAR construct
(SEQ ID NO: 53)
MALPVTALLLPLALLLHAARPMADYKDIVMTQSHKFMSTSVGDRVNITC
KASQNVDSAVAWYQQKPGQSPKALIYSASYRYSGVPDRFTGRGSGTDFT
LTISSVQAEDLAVYYCQQYYSTPWTFGGGTKLEIKRGGGGSGGGGSGGG
GSEVKLVESGGGLVQPGGSLSLSCAASGFTFTDYYMSWVRQPPGKALEW
LALIRSKADGYTTEYSASVKGRFTLSRDDSQSILYLQMNALRPEDSATY
YCARDAAYYSYYSPEGAMDYWGQGTSVTVSSTTTPAPRPPTPAPTIAS

As expected, cells expressing the conventional anti-CD123 CAR construct (anti-CD123 CAR) induced cytotoxicity of wildtype MOLM13 cells (expressing CD123) (reduced % targets alive), whereas the majority of MOLM13 cells deficient in CD123 remained alive (FIG. 5). Similarly, cells expressing an exemplary IL3-zetakine (WT-No costim) induced cytotoxicity of wildtype MOLM13 cells (expressing CD123) (reduced % targets alive), whereas the increased levels of MOLM13 cells deficient in CD123 remained alive (FIG. 5). These results indicate the IL3-zetakines were effective in targeting CD123-expressing cells and activating the cells to induce effector functions, such as cytotoxicity.

### DISCUSSION

This work supports IL3-zetakines described herein as an effective alternatives to conventional CD123-targeted CAR constructs. These results indicate the ability of IL3-zetakine-expressing T cells to kill CD123-expressing AML cells and illustrate the potential of this novel class of therapeutics.

An exemplary nucleotide sequence for the construct shown in FIG 11B is provided by SEQ
ID NO: 54.
(SEQ ID NO: 54)
GGGTCTCTCTGGTTAGACCAGATCTGAGCCTGGGAGCTCTCTGGCTAACTAGGGAACCCACTGCTTAAGCCTCAA
TAAAGCTTGCCTTGAGTGCTTCAAGTAGTGTGTGCCCGTCTGTTGTGTGACTCTGGTAACTAGAGATCCCTCAGA
CCCTTTTAGTCAGTGTGGAAAATCTCTAGCAGTGGCGCCCGAACAGGGACTTGAAAGCGAAAGGGAAACCAGAGG
AGCTCTCTCGACGCAGGACTCGGCTTGCTGAAGCGCGCACGGCAAGAGGCGAGGGGCGGCGACTGGTGAGTACGC
CAAAAATTTTGACTAGCGGAGGCTAGAAGGAGAGAGATGGGTGCGAGAGCGTCAGTATTAAGCGGGGGAGAATTA
GATCGCGATGGGAAAAAATTCGGTTAAGGCCAGGGGGAAAGAAAAAATATAAATTAAAACATATAGTATGGGCAA
GCAGGGAGCTAGAACGATTCGCAGTTAATCCTGGCCTGTTAGAAACATCAGAAGGCTGTAGACAAATACTGGGAC
AGCTACAACCATCCCTTCAGACAGGATCAGAAGAACTTAGATCATTATATAATACAGTAGCAACCCTCTATTGTG
TGCATCAAAGGATAGAGATAAAAGACACCAAGGAAGCTTTAGACAAGATAGAGGAAGAGCAAAACAAAAGTAAGA
CCACCGCACAGCAAGCGGCCACTGATCTTCAGACCTGGAGGAGGAGATATGAGGGACAATTGGAGAAGTGAATTA
TATAAATATAAAGTAGTAAAAATTGAACCATTAGGAGTAGCACCCACCAAGGCAAAGAGAAGAGTGGTGCAGAGA
GAAAAAAGAGCAGTGGGAATAGGAGCTTTGTTCCTTGGGTTCTTGGGAGCAGCAGGAAGCACTATGGGCGCAGCG
TCAATGACGCTGACGGTACAGGCCAGACAATTATTGTCTGGTATAGTGCAGCAGCAGAACAATTTGCTGAGGGCT
ATTGAGGCGCAACAGCATCTGTTGCAACTCACAGTCTGGGGCATCAAGCAGCTCCAGGCAAGAATCCTGGCTGTG
GAAAGATACCTAAAGGATCAACAGCTCCTGGGGATTTGGGGTTGCTCTGGAAAACTCATTTGCACCACTGCTGTG
CCTTGGAATGCTAGTTGGAGTAATAAATCTCTGGAACAGAATTGGAATCACACGACCTGGATGGAGTGGGACAGA
GAAATTAACAATTACACAAGCTTAATACACTCCTTAATTGAAGAATCGCAAAACCAGCAAGAAAAGAATGAACAA
GAATTATTGGAATTAGATAAATGGGCAAGTTTGTGGAATTGGTTTAACATAACAAATTGGCTGTGGTATATAAAA
TTATTCATAATGATAGTAGGAGGCTTGGTAGGTTTAAGAATAGTTTTTGCTGTACTTTCTATAGTGAATAGAGTT
AGGCAGGGATATTCACCATTATCGTTTCAGACCCACCTCCCAACCCCGAGGGGACCCGACAGGCCCGAAGGAATA
GAAGAAGAAGGTGGAGAGAGAGACAGAGACAGATCCATTCGATTAGTGAACGGATCTCGACGGTATCGGTTAACT
TTTAAAAGAAAAGGGGGGATTGGGGGGTACAGTGCAGGGGAAAGAATAGTAGACATAATAGCAACAGACATACAA
ACTAAAGAATTACAAAAACAAATTACAAAATTCAAAATTTTATCGATACTAGTGGATCTGCGATCGCGTAACGCC
ATTTTGCAAGGCATGGAAAAATACCAAACCAAGAATAGAGAAGTTCAGATCAAGGGCGGGTACATGAAAATAGCT
AACGTTGGGCCAAACAGGATATCTGCGGTGAGCAGTTTCGGCCCCGGCCCGGGGCCAAGAACAGATGGTCACCGC
AGTTTCGGCCCCGGCCCGAGGCCAAGAACAGATGGTCCCCAGATATGGCCCAACCCTCAGCAGTTTCTTAAGACC
CATCAGATGTTTCCAGGCTCCCCCAAGGACCTGAAATGACCCTGCGCCTTATTTGAATTAACCAATCAGCCTGCT
TCTCGCTTCTGTTCGCGCGCTTCTGCTTCCCGAGCTCTATAAAAGAGCTCACAACCCCTCACTCGGCGCGCCAGT
CCTCCGACAGACTGAGTCGTCTAGAGCTAGCGGATCCCCGGAATTCGACGCCACCATGCTGCTGCTGGTGACCAG
CCTGCTGCTGTGCGAGCTGCCCCACCCCGCCTTCCTGCTGATCGTGAACTGCAGCAACATGATCGACGAGATCAT
CACCCACCTGAAGCAGCCCCCCCTGCCCCTGCTGGACTTCAACAACCTGAACGGCGAGGACCAGGACATCCTGAT
GGAGAACAACCTGAGGAGGCCCAACCTGGAGGCCTTCAACAGGGCCGTGAAGAGCCTGCAGAACGCCAGCGCCAT
CGAGAGCATCCTGAAGAACCTGCTGCCCTGCCTGCCCCTGGCCACCGCCGCCCCCACCAGGCACCCCATCCACAT
CAAGGACGGCGACTGGAACGAGTTCAGGAGGAAGCTGACCTTCTACCTGAAGACCCTGGAGGAGAGCAAGTACGG
CCCCCCCTGCCCCCCCTGCCCCGCCCCCGAGTTCCTGGGCGGCCCCAGCGTGTTCCTGTTCCCCCCCAAGCCCAA
GGACACCCTGATGATCAGCAGGACCCCCGAGGTGACCTGCGTGGTGGTGGACGTGAGCCAGGAGGACCCCGAGGT
GCAGTTCAACTGGTACGTGGACGGCGTGGAGGTGCACAACGCCAAGACCAAGCCCAGGGAGGAGCAGTTCAACAG
CACCTACAGGGTGGTGAGCGTGCTGACCGTGCTGCACCAGGACTGGCTGAACGGCAAGGAGTACAAGTGCAAGGT
GAGCAACAAGGGCCTGCCCAGCAGCATCGAGAAGACCATCAGCAAGGCCAAGGGCCAGCCCAGGGAGCCCCAGGT
GTACACCCTGCCCCCCAGCCAGGAGGAGATGACCAAGAACCAGGTGAGCCTGACCTGCCTGGTGAAGGGCTTCTA
CCCCAGCGACATCGCCGTGGAGTGGGAGAGCAACGGCCAGCCCGAGAACAACTACAAGACCACCCCCCCCGTGCT
GGACAGCGACGGCAGCTTCTTCCTGTACAGCAGGCTGACCGTGGACAAGAGCAGGTGGCAGGAGGGCAACGTGTT
CAGCTGCAGCGTGATGCACGAGGCCCTGCACAACCACTACACCCAGAAGAGCCTGAGCCTGAGCCTGGGCAAGAT
GGCCCTGATCGTGCTGGGCGGCGTGGCCGGCCTGCTGCTGTTCATCGGCCTGGGCATCTTCTTCAGGGTGAAGTT
CAGCAGGAGCGCCGACGCCCCCGCCTACCAGCAGGGCCAGAACCAGCTGTACAACGAGCTGAACCTGGGCAGGAG
GGAGGAGTACGACGTGCTGGACAAGAGGAGGGGCAGGGACCCCGAGATGGGCGGCAAGCCCAGGAGGAAGAACCC
CCAGGAGGGCCTGTACAACGAGCTGCAGAAGGACAAGATGGCCGAGGCCTACAGCGAGATCGGCATGAAGGGCGA
GAGGAGGAGGGGCAAGGGCCACGACGGCCTGTACCAGGGCCTGAGCACCGCCACCAAGGACACCTACGACGCCCT
GCACATGCAGGCCCTGCCCCCCAGGGAGGGCAGAGGAAGTCTTCTAACATGCGGTGACGTGGAGGAGAATCCCGG
CCCTTCCGGGATGACCGAGTACAAGCCCACGGTGCGCCTCGCCACCCGCGACGACGTCCCCAGGGCCGTACGCAC
CCTCGCCGCCGCGTTCGCCGACTACCCCGCCACGCGCCACACCGTCGATCCGGACCGCCACATCGAGCGGGTCAC
CGAGCTGCAAGAACTCTTCCTCACGCGCGTCGGGCTCGACATCGGCAAGGTGTGGGTCGCGGACGACGGCGCCGC
GGTGGCGGTCTGGACCACGCCGGAGAGCGTCGAAGCGGGGGCGGTGTTCGCCGAGATCGGCCCGCGCATGGCCGA
GTTGAGCGGTTCCCGGCTGGCCGCGCAGCAACAGATGGAAGGCCTCCTGGCGCCGCACCGGCCCAAGGAGCCCGC
GTGGTTCCTGGCCACCGTCGGCGTCTCGCCCGACCACCAGGGCAAGGGTCTGGGCAGCGCCGTCGTGCTCCCCGG
AGTGGAGGCGGCCGAGCGCGCCGGGGTGCCCGCCTTCCTGGAGACCTCCGCGCCCCGCAACCTCCCCTTCTACGA
GCGGCTCGGCTTCACCGTCACCGCCGACGTCGAGGTGCCCGAAGGACCGCGCACCTGGTGCATGACCCGCAAGCC
CGGTGCCTGAATTAATTAACCAATTGAATCAACCTCTGGATTACAAAATTTGTGAAAGATTGACTGGTATTCTTA
ACTATGTTGCTCCTTTTACGCTATGTGGATACGCTGCTTTAATGCCTTTGTATCATGCTATTGCTTCCCGTATGG
CTTTCATTTTCTCCTCCTTGTATAAATCCTGGTTGCTGTCTCTTTATGAGGAGTTGTGGCCCGTTGTCAGGCAAC
GTGGCGTGGTGTGCACTGTGTTTGCTGACGCAACCCCCACTGGTTGGGGCATTGCCACCACCTGTCAGCTCCTTT
CCGGGACTTTCGCTTTCCCCCTCCCTATTGCCACGGCGGAACTCATCGCCGCCTGCCTTGCCCGCTGCTGGACAG
GGGCTCGGCTGTTGGGCACTGACAATTCCGTGGTGTTGTCGGGGAAATCATCGTCCTTTCCTTGGCTGCTCGCCT
GTGTTGCCACCTGGATTCTGCGCGGGACGTCCTTCTGCTACGTCCCTTCGGCCCTCAATCCAGCGGACCTTCCTT
CCCGCGGCCTGCTGCCGGCTCTGCGGCCTCTTCCGCGTCTTCGCCTTCGCCCTCAGACGAGTCGGATCTCCCTTT
GGGCCGCCTCCCCGCCTGGTACCTTTAAGACCAATGACTTACAAGGCAGCTGTAGATCTTAGCCACTTTTTAAAA
GAAAAGGGGGGACTGGAAGGGCTAATTCACTCCCAACGAAAATAAGATCTGCTTTTTGCTTGTACTGGGTCTCTC
TGGTTAGACCAGATCTGAGCCTGGGAGCTCTCTGGCTAACTAGGGAACCCACTGCTTAAGCCTCAATAAAGCTTG
CCTTGAGTGCTTCAAGTAGTGTGTGCCCGTCTGTTGTGTGACTCTGGTAACTAGAGATCCCTCAGACCCTTTTAG
TCAGTGTGGAAAATCTCTAGCA
An exemplary nucleotide sequence for the construct shown in FIG 12B is provided by SEQ ID NO:
55.
(SEQ ID NO: 55)
GGGTCTCTCTGGTTAGACCAGATCTGAGCCTGGGAGCTCTCTGGCTAACTAGGGAACCCACTGCTTAAGCCTCAA
TAAAGCTTGCCTTGAGTGCTTCAAGTAGTGTGTGCCCGTCTGTTGTGTGACTCTGGTAACTAGAGATCCCTCAGA
CCCTTTTAGTCAGTGTGGAAAATCTCTAGCAGTGGCGCCCGAACAGGGACTTGAAAGCGAAAGGGAAACCAGAGG
AGCTCTCTCGACGCAGGACTCGGCTTGCTGAAGCGCGCACGGCAAGAGGCGAGGGGCGGCGACTGGTGAGTACGC
CAAAAATTTTGACTAGCGGAGGCTAGAAGGAGAGAGATGGGTGCGAGAGCGTCAGTATTAAGCGGGGGAGAATTA
GATCGCGATGGGAAAAAATTCGGTTAAGGCCAGGGGGAAAGAAAAAATATAAATTAAAACATATAGTATGGGCAA
GCAGGGAGCTAGAACGATTCGCAGTTAATCCTGGCCTGTTAGAAACATCAGAAGGCTGTAGACAAATACTGGGAC
AGCTACAACCATCCCTTCAGACAGGATCAGAAGAACTTAGATCATTATATAATACAGTAGCAACCCTCTATTGTG
TGCATCAAAGGATAGAGATAAAAGACACCAAGGAAGCTTTAGACAAGATAGAGGAAGAGCAAAACAAAAGTAAGA
CCACCGCACAGCAAGCGGCCACTGATCTTCAGACCTGGAGGAGGAGATATGAGGGACAATTGGAGAAGTGAATTA
TATAAATATAAAGTAGTAAAAATTGAACCATTAGGAGTAGCACCCACCAAGGCAAAGAGAAGAGTGGTGCAGAGA
GAAAAAAGAGCAGTGGGAATAGGAGCTTTGTTCCTTGGGTTCTTGGGAGCAGCAGGAAGCACTATGGGCGCAGCG
TCAATGACGCTGACGGTACAGGCCAGACAATTATTGTCTGGTATAGTGCAGCAGCAGAACAATTTGCTGAGGGCT
ATTGAGGCGCAACAGCATCTGTTGCAACTCACAGTCTGGGGCATCAAGCAGCTCCAGGCAAGAATCCTGGCTGTG
GAAAGATACCTAAAGGATCAACAGCTCCTGGGGATTTGGGGTTGCTCTGGAAAACTCATTTGCACCACTGCTGTG
CCTTGGAATGCTAGTTGGAGTAATAAATCTCTGGAACAGAATTGGAATCACACGACCTGGATGGAGTGGGACAGA
GAAATTAACAATTACACAAGCTTAATACACTCCTTAATTGAAGAATCGCAAAACCAGCAAGAAAAGAATGAACAA
GAATTATTGGAATTAGATAAATGGGCAAGTTTGTGGAATTGGTTTAACATAACAAATTGGCTGTGGTATATAAAA
TTATTCATAATGATAGTAGGAGGCTTGGTAGGTTTAAGAATAGTTTTTGCTGTACTTTCTATAGTGAATAGAGTT
AGGCAGGGATATTCACCATTATCGTTTCAGACCCACCTCCCAACCCCGAGGGGACCCGACAGGCCCGAAGGAATA
GAAGAAGAAGGTGGAGAGAGAGACAGAGACAGATCCATTCGATTAGTGAACGGATCTCGACGGTATCGGTTAACT
TTTAAAAGAAAAGGGGGGATTGGGGGGTACAGTGCAGGGGAAAGAATAGTAGACATAATAGCAACAGACATACAA
ACTAAAGAATTACAAAAACAAATTACAAAATTCAAAATTTTATCGATACTAGTGGATCTGCGATCGCGTAACGCC
ATTTTGCAAGGCATGGAAAAATACCAAACCAAGAATAGAGAAGTTCAGATCAAGGGCGGGTACATGAAAATAGCT
AACGTTGGGCCAAACAGGATATCTGCGGTGAGCAGTTTCGGCCCCGGCCCGGGGCCAAGAACAGATGGTCACCGC
AGTTTCGGCCCCGGCCCGAGGCCAAGAACAGATGGTCCCCAGATATGGCCCAACCCTCAGCAGTTTCTTAAGACC
CATCAGATGTTTCCAGGCTCCCCCAAGGACCTGAAATGACCCTGCGCCTTATTTGAATTAACCAATCAGCCTGCT
TCTCGCTTCTGTTCGCGCGCTTCTGCTTCCCGAGCTCTATAAAAGAGCTCACAACCCCTCACTCGGCGCGCCAGT
CCTCCGACAGACTGAGTCGTCTAGAGCTAGCGGATCCCCGGAATTCGACGCCACCATGCTGCTGCTGGTGACCAG
CCTGCTGCTGTGCGAGCTGCCCCACCCCGCCTTCCTGCTGATCGTGAACTGCAGCAACATGATCGACGAGATCAT
CACCCACCTGAAGCAGCCCCCCCTGCCCCTGCTGGACTTCAACAACCTGAACGGCGAGGACCAGGACATCCTGAT
GGAGAACAACCTGAGGAGGCCCAACCTGGAGGCCTTCAACAGGGCCGTGAAGAGCCTGCAGAACGCCAGCGCCAT
CGAGAGCATCCTGAAGAACCTGCTGCCCTGCCTGCCCCTGGCCACCGCCGCCCCCACCAGGCACCCCATCCACAT
CAAGGACGGCGACTGGAACGAGTTCAGGAGGAAGCTGACCTTCTACCTGAAGACCCTGGAGGAGAGCAAGTACGG
CCCCCCCTGCCCCCCCTGCCCCGCCCCCGAGTTCCTGGGCGGCCCCAGCGTGTTCCTGTTCCCCCCCAAGCCCAA
GGACACCCTGATGATCAGCAGGACCCCCGAGGTGACCTGCGTGGTGGTGGACGTGAGCCAGGAGGACCCCGAGGT
GCAGTTCAACTGGTACGTGGACGGCGTGGAGGTGCACAACGCCAAGACCAAGCCCAGGGAGGAGCAGTTCAACAG
CACCTACAGGGTGGTGAGCGTGCTGACCGTGCTGCACCAGGACTGGCTGAACGGCAAGGAGTACAAGTGCAAGGT
GAGCAACAAGGGCCTGCCCAGCAGCATCGAGAAGACCATCAGCAAGGCCAAGGGCCAGCCCAGGGAGCCCCAGGT
GTACACCCTGCCCCCCAGCCAGGAGGAGATGACCAAGAACCAGGTGAGCCTGACCTGCCTGGTGAAGGGCTTCTA
CCCCAGCGACATCGCCGTGGAGTGGGAGAGCAACGGCCAGCCCGAGAACAACTACAAGACCACCCCCCCCGTGCT
GGACAGCGACGGCAGCTTCTTCCTGTACAGCAGGCTGACCGTGGACAAGAGCAGGTGGCAGGAGGGCAACGTGTT
CAGCTGCAGCGTGATGCACGAGGCCCTGCACAACCACTACACCCAGAAGAGCCTGAGCCTGAGCCTGGGCAAGAT
GGCCCTGATCGTGCTGGGCGGCGTGGCCGGCCTGCTGCTGTTCATCGGCCTGGGCATCTTCTTCAAGAGGGGCAG
GAAGAAGCTGCTGTACATCTTCAAGCAGCCCTTCATGAGGCCCGTGCAGACCACCCAGGAGGAGGACGGCTGCAG
CTGCAGGTTCCCCGAGGAGGAGGAGGGCGGCTGCGAGCTGAGGGTGAAGTTCAGCAGGAGCGCCGACGCCCCCGC
CTACCAGCAGGGCCAGAACCAGCTGTACAACGAGCTGAACCTGGGCAGGAGGGAGGAGTACGACGTGCTGGACAA
GAGGAGGGGCAGGGACCCCGAGATGGGCGGCAAGCCCAGGAGGAAGAACCCCCAGGAGGGCCTGTACAACGAGCT
GCAGAAGGACAAGATGGCCGAGGCCTACAGCGAGATCGGCATGAAGGGCGAGAGGAGGAGGGGCAAGGGCCACGA
CGGCCTGTACCAGGGCCTGAGCACCGCCACCAAGGACACCTACGACGCCCTGCACATGCAGGCCCTGCCCCCCAG
GGAGGGCAGAGGAAGTCTTCTAACATGCGGTGACGTGGAGGAGAATCCCGGCCCTTCCGGGATGACCGAGTACAA
GCCCACGGTGCGCCTCGCCACCCGCGACGACGTCCCCAGGGCCGTACGCACCCTCGCCGCCGCGTTCGCCGACTA
CCCCGCCACGCGCCACACCGTCGATCCGGACCGCCACATCGAGCGGGTCACCGAGCTGCAAGAACTCTTCCTCAC
GCGCGTCGGGCTCGACATCGGCAAGGTGTGGGTCGCGGACGACGGCGCCGCGGTGGCGGTCTGGACCACGCCGGA
GAGCGTCGAAGCGGGGGCGGTGTTCGCCGAGATCGGCCCGCGCATGGCCGAGTTGAGCGGTTCCCGGCTGGCCGC
GCAGCAACAGATGGAAGGCCTCCTGGCGCCGCACCGGCCCAAGGAGCCCGCGTGGTTCCTGGCCACCGTCGGCGT
CTCGCCCGACCACCAGGGCAAGGGTCTGGGCAGCGCCGTCGTGCTCCCCGGAGTGGAGGCGGCCGAGCGCGCCGG
GGTGCCCGCCTTCCTGGAGACCTCCGCGCCCCGCAACCTCCCCTTCTACGAGCGGCTCGGCTTCACCGTCACCGC
CGACGTCGAGGTGCCCGAAGGACCGCGCACCTGGTGCATGACCCGCAAGCCCGGTGCCTGAATTAATTAACCAAT
TGAATCAACCTCTGGATTACAAAATTTGTGAAAGATTGACTGGTATTCTTAACTATGTTGCTCCTTTTACGCTAT
GTGGATACGCTGCTTTAATGCCTTTGTATCATGCTATTGCTTCCCGTATGGCTTTCATTTTCTCCTCCTTGTATA
AATCCTGGTTGCTGTCTCTTTATGAGGAGTTGTGGCCCGTTGTCAGGCAACGTGGCGTGGTGTGCACTGTGTTTG
CTGACGCAACCCCCACTGGTTGGGGCATTGCCACCACCTGTCAGCTCCTTTCCGGGACTTTCGCTTTCCCCCTCC
CTATTGCCACGGCGGAACTCATCGCCGCCTGCCTTGCCCGCTGCTGGACAGGGGCTCGGCTGTTGGGCACTGACA
ATTCCGTGGTGTTGTCGGGGAAATCATCGTCCTTTCCTTGGCTGCTCGCCTGTGTTGCCACCTGGATTCTGCGCG
GGACGTCCTTCTGCTACGTCCCTTCGGCCCTCAATCCAGCGGACCTTCCTTCCCGCGGCCTGCTGCCGGCTCTGC
GGCCTCTTCCGCGTCTTCGCCTTCGCCCTCAGACGAGTCGGATCTCCCTTTGGGCCGCCTCCCCGCCTGGTACCT
TTAAGACCAATGACTTACAAGGCAGCTGTAGATCTTAGCCACTTTTTAAAAGAAAAGGGGGGACTGGAAGGGCTA
ATTCACTCCCAACGAAAATAAGATCTGCTTTTTGCTTGTACTGGGTCTCTCTGGTTAGACCAGATCTGAGCCTGG
GAGCTCTCTGGCTAACTAGGGAACCCACTGCTTAAGCCTCAATAAAGCTTGCCTTGAGTGCTTCAAGTAGTGTGT
GCCCGTCTGTTGTGTGACTCTGGTAACTAGAGATCCCTCAGACCCTTTTAGTCAGTGTGGAAAATCTCTAGCA

### Example 2: Specific Activation and Cytotoxic Targeting of CD123+ Cells

### Expression and Activation of Jurkat Cells

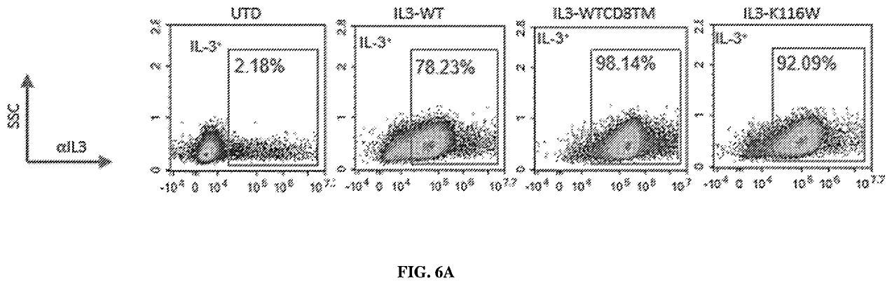

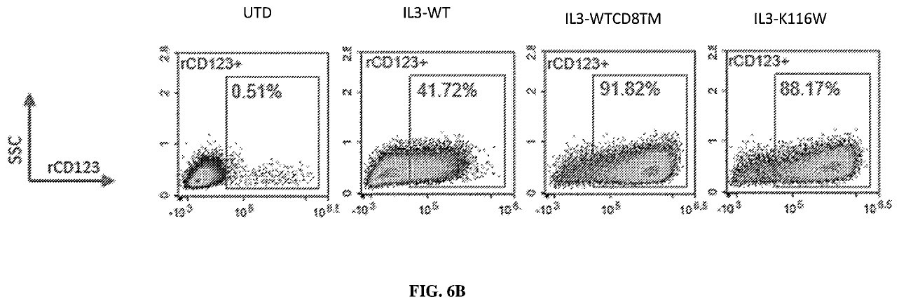

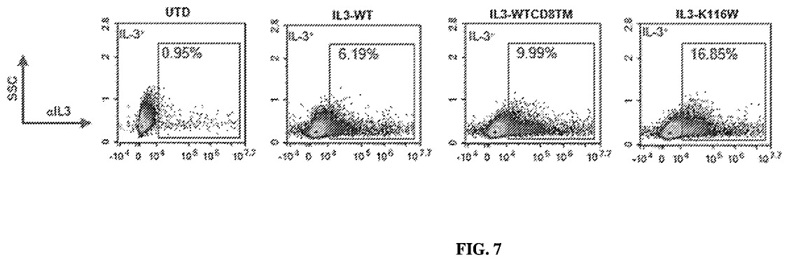

Exemplary zetakine constructs were transduced into TIB-153™ Jurkat cells as described in Example 1. Expression of the IL3-zetakine constructs was assessed by staining cells with anti-IL3 antibody or biotinylated recombinant CD123 protein bound to fluorescence-tagged streptavidin. See, FIGS. 6A 6B. Both staining methods indicated that the IL3-zetakines were expressed in up to 98% of TIB-153™ Jurkat cells.

TIB-153™ Jurkat cells transduced with IL3-zetakines were co-cultured at a 1:1 ratio of effector to target cells for 24 hours with a CD123-null HL60-WT cell line, or an AML cell line expressing low levels of CD123 (MOLM13-CD123_{low}), or wild-type MOLM13 cells (MOLM13-WT; CD123 high), or cultured in the absence of target cells.

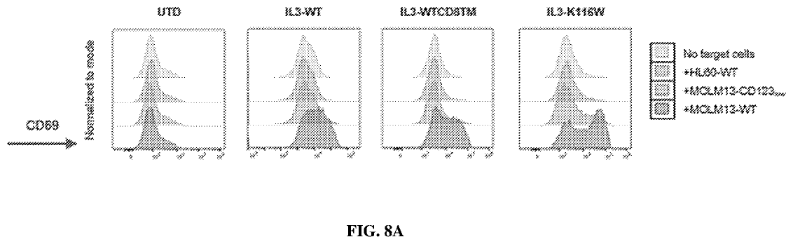

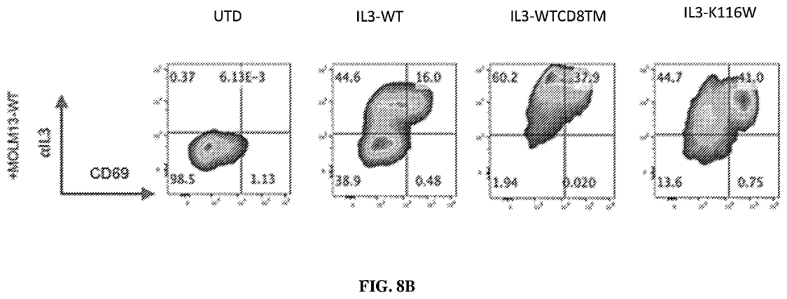

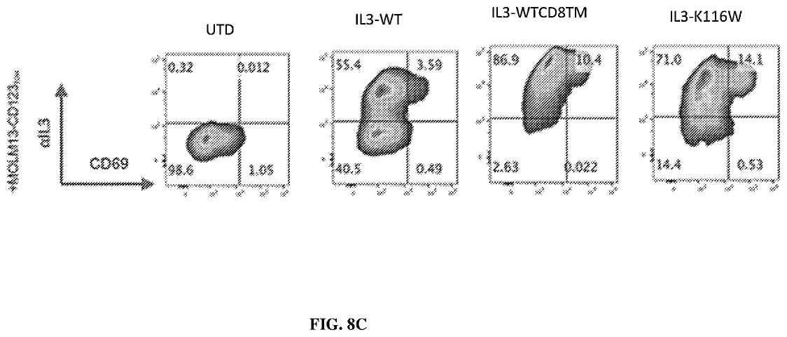

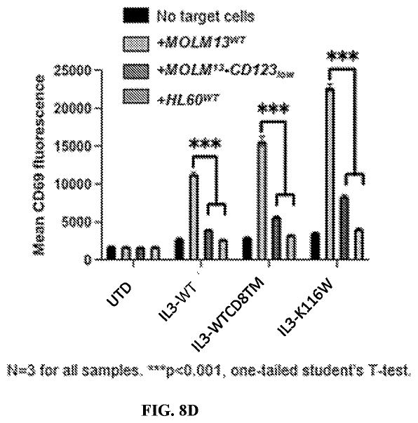

The mean fluorescence intensity (MFI) of CD69 expression, a marker of T cell activation, was measured as a function of zetakine expression (detected by staining with an anti-IL3 antibody). Sec, FIGS. 8A-8D. Significant cell activation (by CD69 expression) was observed for cells expressing the IL3 zetakines when co-cultured in the presence of MOLM13-WT cells (CD123 high), and lower but still detectable when co-cultured in the presence of MOLM13-CD123_{low} cells, as compared to co-culturing with HL60-WT cells (CD123-null) or the absence of target cells.

### Expression and Activation of Primary T Cells

To assess transduction of IL3-zetakine in primary human peripheral blood mononuclear cells (PBMCs), PBMCs were treated with Transact (containing CD3/CD28 containing activation matrix) prior to transduction with lentivirus constructs carrying IL3-zetakine sequences, left untransduced (UTD). After 7 days post-transduction, flow cytometry analyses pregated on CD3+ T cells and expression of the IL3-zetakines were detected using an anti-IL3 antibody. As shown in FIG. 7, expression levels of the IL3-zetakines varied across transduction conditions in a range from 6%-16% of CD3+ T cells.

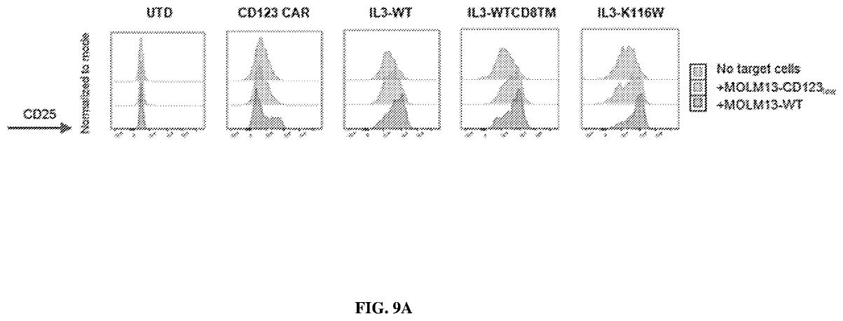

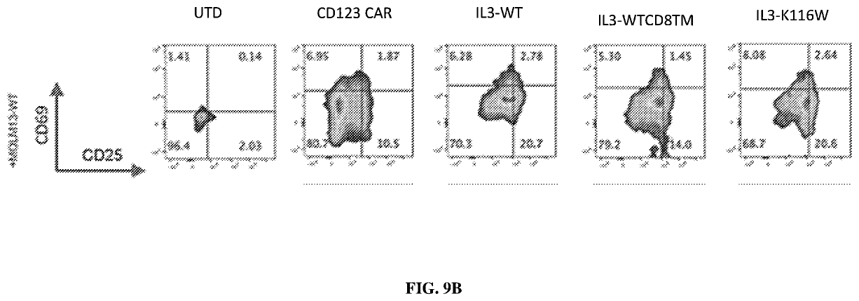

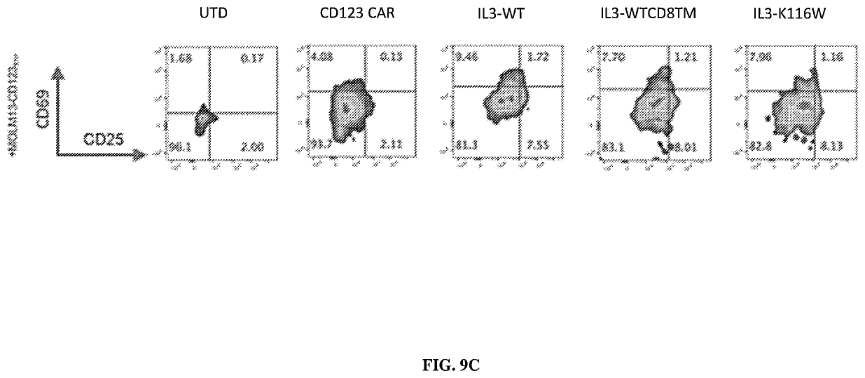

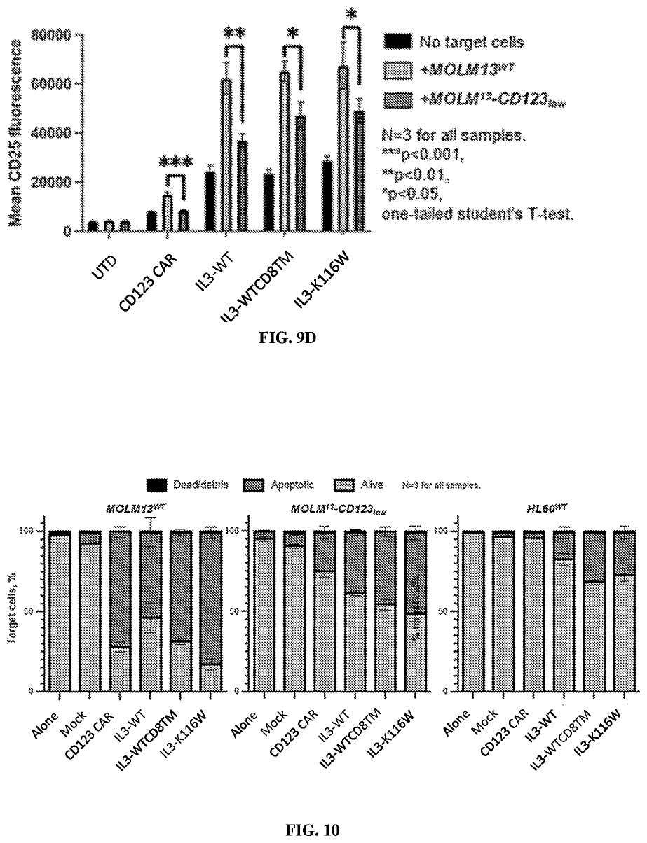

Activation of primary T cells expressing the IL3 zetakines was assessed by measuring both CD25 and CD69 expression via flow cytometry of sorted IL3-zetakine-positive PBMCs after co-culture with target cells. Briefly, primary cells transduced with IL3-zetakines (or an anti-CD123 CAR control) were co-cultured with wild-type MOLM13 cells (MOLM13-WT; CD123 high) or MOLM13-CD123_{low} cells (also referred to as MOM13^{MUT}) at a 1:1 ratio of effector to target cells for 24 hours. The mean fluorescence intensity (MFI) of CD25 expression on CD3+ T cells was measured (FIG. 9A). FIGS. 9B and 9C show the level of IL3-zetakine-ineduced activation, indicated by both CD25 and CD69 expression of CD3+ cells co-cultured with the indicated target cells. Co-culturing of IL3-zetakine-expressing primary human PBMCs with MOLM13-WT cells resulted in increases in CD25 and CD69 expression levels (FIG. 9A-9D). Significant cell activation (by CD25 expression) was observed for cells expressing the IL3 zetakines when co-cultured in the presence of MOLM13-WT cells (CD123 high), and lower but still detectable when co-cultured in the presence of MOLM13-CD123_{low} cells, as compared to the absence of target cells. FIG. 9D.

### Cytotoxicity of Primary T Cells Expressing IL3-Zetakines

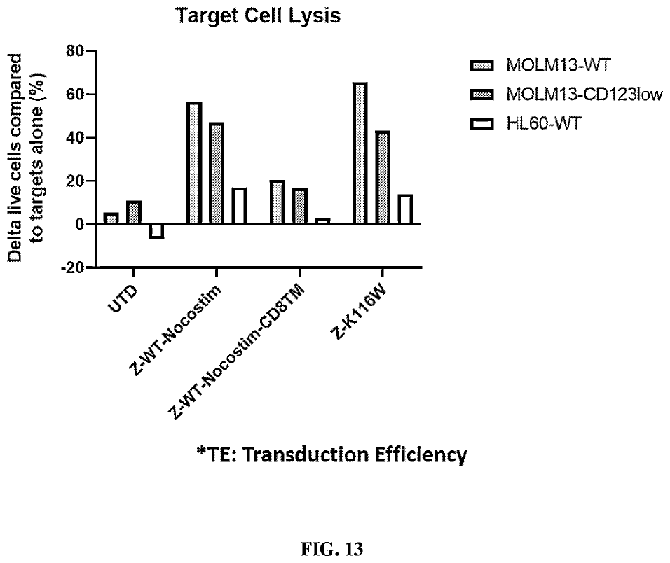

IL-3 zetakines were tested for their ability to induce target cell cytotoxicity in vitro. CD123-expressing target cells (MOLM13-WT, MOLM13-CD123_{low}) and CD123 null cells (HL60-WT) were seeded at a density of 100,000 target cells per well and were either untransduced (UTD) or transduced with zetakines IL3-WT-nocostim (SEQ ID NO: 21), IL3-WTCD8TM (SEQ ID NO: 33), or IL3-K116W (SEQ ID NO: 51) during a 24-hour co-culturing period at an effector to target cell ratio of 1:1, normalized to transduction efficiency (TE). Cell lysis was assessed relative to target cells alone. CD123 expression levels correlated with higher levels of cell lysis as a result of IL-3 zetakine transduction with MOLM13-WT cells exhibiting the highest amounts of cell lysis and HL60-WT cells exhibiting the lowest, indicating the cytotoxicity is CD123 specific (FIG. 13). These results indicated that IL-3 zetakines exhibited in vitro cytotoxicity in CD123-expressing target cells.

Primary cells expressing IL3-zetakine constructs, or an anti-CD123 CAR construct as a positive control, were also assessed for their ability to induce cytotoxicity of CD123-expressing target cells. Cells were co-cultured with wild-type MOLM13 cells (MOLM13-WT; CD123 high) or MOLM13-CD123_{low} cells, or CD123-null HL60-WT cells at a 1:1 ratio of effector to target cells for 24 hours. Target cell health was assessed by flow cytometry. Alive cells were viable and Annexin V-negative; apoptotic cells were viability negative and Annexin V-positive; and dead cells/debris were viability and Annexin V-positive.

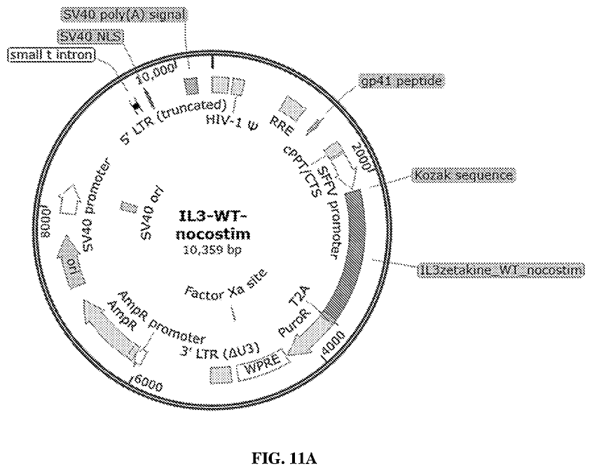

Primary cells expressing IL3-WT-nocostim, IL3-WTCD8TM-nocostim, or IL3-K116W-nocostim co-cultured with MOLM13 cells expressing CD123 resulted in increased apoptotic markers, and fewer live cells, relative to co-culturing with MOLM13-CD123_{low} or HL60-WT cells (CD123 null) (FIG. 10). The bar graph indicated that IL3-zetakine-expressing T cells induced target cell cytotoxicity to a similar or greater extent as the CD123 CAR-expressing cells.

These results indicate the IL3-zetakines were activated in the presence of CD123-expressing target cells and activation induced effector functions, such as cytotoxicity.

### REFERENCES

1. Ganzel, C., et al., Very poor long-term survival in past and more recent studies for relapsed AML patients: The ECOG-ACRIN experience. American journal of hematology, 2018: p. 10.1002/ajh.25162.
2. Shlush, L. I., et al., Tracing the origins of relapse in acute myeloid leukaemia to stem cells. Nature, 2017. 547 (7661): p. 104-108.
3. Hanekamp, D., J. Cloos, and G. J. Schuurhuis, Leukemic stem cells: identification and clinical application. International Journal of Hematology, 2017. 105 (5): p. 549-557.
4. Bras, A. E., et al., CD123 expression levels in 846 acute leukemia patients based on standardized immunophenotyping. Cytometry Part B: Clinical Cytometry, 2019. 96 (2): p. 134-142.
5. Sugita, M. and M. L. Guzman, CD123 as a Therapeutic Target Against Malignant Stem Cells. Hematology/Oncology Clinics of North America, 2020. 34 (3): p. 553-564.
6. Mingyue, S., et al., CD123: A Novel Biomarker for Diagnosis and Treatment of Leukemia. Cardiovascular & Hematological Disorders-Drug Targets, 2019. 19 (3): p. 195-204.
7. Kahlon, K. S., et al., Specific recognition and killing of glioblastoma multiforme by interleukin 13-zetakine redirected cytolytic T cells. Cancer Res, 2004. 64 (24): p. 9160-6.
8. Bagley, C. J., et al., A discontinuous eight-amino acid epitope in human interleukin-3 binds the alpha-chain of its receptor. J Biol Chem, 1996. 271 (50): p. 31922-8.
9. Chandrashekar, D. S., et al., UALCAN: A Portal for Facilitating Tumor Subgroup Gene Expression and Survival Analyses. Neoplasia, 2017. 19 (8): p. 649-658.

### REFERENCES

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### Equivalents and Scope

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which two or more members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods described herein, can be excluded from any one or more claims. For purposes of brevity, all of the embodiments in which one or more elements, features, purposes, or aspects is excluded are not set forth explicitly herein.

What is claimed is:

1. A chimeric antigen receptor (CAR), comprising
a. an interleukin-3 (IL-3) molecule or a CD123-binding fragment thereof;
b. optionally, a linker region,
c. optionally, a hinge region,
d. a transmembrane region,
e. optionally, at least one costimulatory signaling domain, and
f. a signaling domain.

2. The CAR of claim 1, wherein the IL-3 molecule, or CD123-binding fragment thereof, comprises a substitution mutation at an amino acid position corresponding to K110, D101, K116, or a combination thereof.

3. The CAR of claim 1 or 2, wherein the IL-3 molecule, or CD123-binding fragment thereof, comprises a D101A mutation, a K116V mutation, a K116W, or a combination thereof.

4. The CAR of any one of claims 1-3, wherein the IL-3 molecule, or CD123-binding fragment thereof, comprises a D101A mutation and a K116V mutation.

5. The CAR of any one of claims 1-4, wherein the signaling domain is a CD3 zeta signaling domain.

6. The CAR of any one of claims 1-5, wherein the CAR comprises at least one costimulatory signaling domain that is CD28, 4-1BB, and/or OX-40 costimulatory signaling domain.

7. The CAR of any one of claims 1-6, wherein the CAR comprises a CD28 costimulatory signaling domain.

8. The CAR of any one of claims 1-7, wherein the CAR comprises a 4-1BB costimulatory signaling domain.

9. The CAR of any one of claims 1-8, wherein the CAR comprises an OX-40 costimulatory signaling domain.

10. The CAR of any one of claims 1-9, wherein the transmembrane region is a human CD4 transmembrane region.

11. The CAR of any one of claims 1-9, wherein the transmembrane region is a human CD8 transmembrane region.

12. The CAR of any one of claims 1-9, wherein the transmembrane region is a human CD28 transmembrane region.

13. The CAR of any one of claims 1-12, wherein the CAR comprises a hinge region that is a human immunoglobulin (Ig) subclass G4 (IgG4) fragment crystallizable (Fc) hinge region.

14. The CAR of any one of claims 1-13, wherein the CAR comprises a linker region that is a human immunoglobulin (Ig) subclass G4 (IgG4) fragment crystallizable (Fc) linker region.

15. The CAR of claim 14, wherein the human IgG4 Fc linker region comprises a substitution mutation at an amino acid position corresponding to L235, N297 or a combination thereof.

16. The CAR of claim 14 or 15, wherein the human IgG4 Fc linker region comprises a L235E mutation, a N297Q mutation, or a combination thereof.

17. The CAR of any one of claims 1-16, wherein the CAR comprises, from N-terminus to C-terminus,
a. the interleukin-3 (IL-3) molecule or CD123-binding fragment thereof;
b. the linker region and/or a hinge region,
c. the transmembrane region, and
d. the signaling domain.

18. The CAR of claim 17, wherein the CAR does not comprise a costimulatory signaling domain.

19. The CAR of claim 17 or 18, wherein the CAR comprises an amino acid sequence having at least 95% sequence identity to any one of SEQ ID NOs: 21, 27, 33, 35, 37, 39, 45, 47, 49, or 51.

20. The CAR of claim 16 or claim 17, wherein the CAR comprises an amino acid sequence of any one of SEQ ID NOs: 21, 27, 33, 35, 37, 39, 45, 47, 49, or 51.

21. The CAR of any one of claims 1-16, wherein the CAR comprises, from N-terminus to C-terminus,
a. the interleukin-3 (IL-3) molecule or a CD123-binding fragment thereof;
b. the linker region and/or a hinge region,
C. the transmembrane region, and
d. the one or more co-stimulatory signaling domains, and
e. the signaling domain.

22. The CAR of claim 21, wherein the CAR comprises an amino acid sequence having at least 95% sequence identity to any one of SEQ ID NOs: 23, 25, 29, 31, 41, or 43.

23. The CAR of claim 21 or claim 22, wherein the CAR comprises an amino acid sequence of any one of SEQ ID NOs: 23, 25, 29, 31, 41, or 43.

24. The CAR of any one of claims 1-23, wherein the CAR further comprises a signal peptide/signal sequence.

25. A nucleic acid construct encoding the CAR of any one of claims 1-24.

26. The nucleic acid construct of claim 25, wherein the nucleic acid construct further comprises a promoter sequence.

27. The nucleic acid construct of claim 25 or 26, wherein the nucleic acid is RNA.

28. The nucleic acid construct of claim 25 or 26, wherein the nucleic acid is DNA.

29. A vector comprising the nucleic acid construct of any one of claims 25-28.

30. The vector of claim 29, wherein the vector is a DNA vector, an RNA vector, a plasmid, a lentivirus vector, an adenoviral vector or a retrovirus vector.

31. A cell comprising the nucleic acid construct of any one of claims 25-30.

32. A cell expressing the CAR of any one of claims 1-24.

33. The cell of claim 31 or claim 32, wherein the cell is an immune effector cell.

34. The cell of claim 33, wherein the cell is a T-lymphocyte.

35. The cell of claim 33, wherein the cell is a NK cell.

36. A pharmaceutical composition comprising the cell of any one of claims 31-35.

37. A method, comprising administering the CAR of any one of claims 1-24, the nucleic acid construct of any one of claims 25-28, the vector of claim 29 or claim 30, or the cell of any one of claims 31-35 to a subject in need thereof.

38. The method of claim 37, wherein the subject has or has been diagnosed with a hematopoietic malignancy or pre-malignancy characterized by the expression of CD123 on malignant cells or pre-malignant cells.

39. The method of claim 38, wherein the hematopoietic malignancy is acute myeloid leukemia (AML).

40. The method of claim 38, wherein the hematopoietic malignancy is myelodysplastic syndrome (MDS).

41. The method of any one of claims 37-40, further comprising administering a population of hematopoietic cells, wherein the hematopoietic cells are genetically-engineered such that the gene encoding CD123 is engineered to reduce or eliminate the expression of CD123.

42. A method comprising introducing into a cell with the nucleic acid construct of any one of claims 25-28, or the vector of claim 29 or claim 30.

43. The method of claim 42, wherein the cell is obtained from, or derived from, a subject prior to introducing the nucleic acid construct or vector.

44. The method of claim 43, wherein the subject has or has been diagnosed with a hematopoietic malignancy or pre-malignancy characterized by the expression of CD123 on malignant cells or pre-malignant cells.

45. The method of any one of claims 42-44, wherein the cell is an immune effector cell.

46. The method of claim 45, wherein the cell is a T-lymphocyte.

47. The method of claim 45, wherein the cell is a NK cell.

48. The method of claim 46 or claim 47, wherein the T lymphocyte or NK cell is activated and/or expanded ex vivo.

49. The method of any one of claims 42-48, wherein the nucleic acid or vector is introduced into the cell by lentiviral transduction, retroviral transduction, adeno-associated viral transduction, DNA electroporation, RNA electroporation, or transposon electroporation.
